(19) United States
(12) Patent Application Publication
(10) Pub. No.: US 2026/0201018 A1
(43) Pub. Date: Jul. 16, 2026

(54) BROADLY NEUTRALIZING ANTIBODY-TEMPLATED DIVALENT IMMUNOGEN VACCINES

(71) Applicant: University of Hawaii, Honolulu, HI (US)

(72) Inventors: Iain MACPHERSON, Honolulu, HI (US); Ryan BAILEY, Honolulu, HI (US); Axel LEHRER, Honolulu, HI (US)

(21) Appl. No.: 19/135,721

(22) PCT Filed: Dec. 4, 2023
(86) PCT No.: PCT/US2023/082376

§ 371 Date: Jun. 4, 2025

Related U.S. Application Data
(60) Provisional application No. 63/430,144, filed on Dec. 5, 2022.

(51) Int. Cl. C07K 16/00; A61K 39/00; A61K 39/21; A61K 39/385; A61K 39/39; C07K 14/16; C12N 15/62; G01N 33/50; G01N 33/53

(52) U.S. Cl. CPC C07K 16/00; A61K 39/21; A61K 39/385; A61K 39/39; C07K 14/162; C12N 15/62; G01N 33/5005; G01N 33/53; A61K 2039/6056; A61K 2039/70; C07K 2317/21; C07K 2317/24; C07K 2317/52; C07K 2317/55; C07K 2317/76; C07K 2318/00; C07K 2319/30

(57) ABSTRACT

The present disclosure relates generally to divalent immunogens comprising a scaffold and two antigens or epitopes thereof, multimers, compositions, and methods of using the same. Such divalent immunogens are useful, for example, for stimulating an immune response.

### CROSS REFERENCE TO RELATED APPLICATIONS

This application claims priority to U.S. Provisional Application No. 63/430,144, filed Dec. 5, 2022, which is incorporated by reference herein in its entirety.

### STATEMENT OF GOVERNMENT INTEREST

This invention was made with government support under NIH Training Grant #5K12HL143960-05 awarded by the National Institutes of Health (NIH). The United States Government, therefore, may have certain rights in the invention.

### TECHNICAL FIELD

The present disclosure relates generally to divalent immunogens comprising a scaffold and two antigens or epitopes thereof, compositions, and methods of using the same. Such divalent immunogens are useful, for example, for stimulating an immune response.

### STATEMENT REGARDING SEQUENCE LISTING

The Sequence Listing XML associated with this application is provided in XML file format and is hereby incorporated by reference into the specification. The name of the XML file containing the Sequence Listing XML is IAMA_001_01WO_ST26.xml. The XML file is 113,944 bytes, was created on Dec. 4, 2023, and is being submitted electronically via USPTO Patent Center.

### BACKGROUND

Vaccine design for rapidly-mutating viruses is challenging, and few successful vaccines have emerged in this area. Protective immune responses against such viruses often requires the elicitation of an immune response characterized by broadly neutralizing antibodies (bnAbs), which can neutralize phenotypically distinct variants of the virus. However, even for viruses for which bnAbs have been identified in patients (e.g., VRC01, a bnAb that binds the CD4 binding site of HIV gp120), eliciting expansion of the B cells that produce these bnAbs has proven difficult.

Expansion of a B cell requires binding of the B-cell receptor (BCR) expressed by that B cell to its cognate antigen. In the context of elicitation of an immune response, B cells expressing BCRs with a higher affinity for the antigen may out-compete those with lower affinity, leading to selective expansion of those with higher affinity. B cells expressing BCRs with a higher affinity for the antigen may survive the affinity maturation process while those with lower-affinity BCRs undergo apoptosis. If a bnAb germline antibody (and its corresponding BCR) has low affinity for its cognate antigen (e.g., VRC01 germline), B cells that express it may not expand sufficiently to generate protection against the virus. Thus, germline B cells must be activated by suitable immunogens to drive the evolution of their receptors toward the bnAb affinity and breadth. In the context of prime+boost vaccine regimens, germline B cells may need to evolve in a preferred direction through multiple bottlenecks in order to generate the preferred BCR (e.g., VRC01-class BCRs). New approaches are needed to selectively elicit expansion of such B cells.

### SUMMARY

In various embodiments, the present disclosure provides divalent immunogens comprising a scaffold and two antigens or epitopes thereof, wherein each antigen or epitope thereof is covalently linked to the scaffold. Also disclosed herein are multimers including these immunogens, pharmaceutical compositions including these immunogens, and polynucleotides encoding at least one antigen and/or epitope of the divalent immunogen, and/or the scaffold or a portion thereof. Also disclosed are methods of identifying antibodies for divalent immunogens described herein, and methods of use of divalent immunogens disclosed herein.

In one embodiment, the disclosure provides a divalent immunogen comprising: a scaffold, and two antigens or epitopes thereof; wherein each antigen or epitope thereof is covalently linked to the scaffold. In some embodiments, only a fraction of antigen-reactive B-cell receptors are able to bind to both antigens or epitopes thereof simultaneously and with 1:1 stoichiometry.

In some embodiments, the scaffold is derived from an antibody. In other embodiments, the scaffold is derived from a dimeric protein or a multimeric protein. In still other embodiments, the scaffold is derived from a monomeric protein. In some embodiments, each antigen or epitope thereof is expressed as a fusion polypeptide comprising at least a part or fragment of the scaffold. In some embodiments, the scaffold is a nucleic acid nanostructure.

In some embodiments, the scaffold comprises an antibody or fragment or variant thereof. In some embodiments, the scaffold comprises two Fab fragments or variants thereof. In some embodiments, the covalent linkage of the antigen or epitope thereof to the scaffold is a disulfide bond. In other embodiments, the covalent linkage is a chemical crosslinker, a protein crosslinker, or a nucleic acid crosslinker.

In some embodiments, each Fab fragment comprises a first engineered Fab mutation that enables formation of the disulfide bond between the Fab fragment and the antigen or epitope thereof. In some embodiments, each Fab fragment comprises a second engineered Fab mutation that enables Fab-Fab crosslinking, wherein the second engineered Fab mutation is in the light chain of each of the two Fab fragments. In some embodiments, each Fab fragment comprises a second engineered Fab mutation that enables Fab-Fab crosslinking, wherein the second engineered Fab mutation is in the heavy chain of each of the two Fab fragments. In some embodiments, the second engineered Fab mutation places the antigens or epitopes thereof in a more favorable orientation for divalent binding of at least one antibody.

In some embodiments, the divalent immunogen comprises an engineered dimerization interface comprising C-terminal extension of the Fab heavy and/or light chains, optionally wherein the engineered dimerization interface places the antigens or epitopes thereof in a more favorable orientation for divalent binding of at least one BCR and/or antibody.

In some embodiments, the divalent immunogen comprises a linker between the two antigens or epitopes thereof, optionally wherein the linker places the antigens or epitopes thereof in a more favorable orientation for divalent binding of at least one BCR and/or antibody.

In some embodiments, the first antigen or epitope thereof is at least 70%, at least 80%, at least 90%, at least 95%, or at least 98% identical to the second antigen or epitope thereof. In some embodiments, the first antigen or epitope thereof is 100% identical to the second antigen or epitope thereof.

In some embodiments, each antigen or epitope thereof comprises a first engineered antigen mutation or a first engineered epitope mutation that enables formation of a disulfide bond between the scaffold and the antigen or epitope thereof.

In some embodiments, the divalent immunogen additionally comprises an Fc domain. In some embodiments, the Fc is a mouse Fc, optionally a mouse IgG2a Fc. In some embodiments, the Fc is a human Fc, optionally a human IgG1 Fc.

In some embodiments, the divalent immunogen comprises an engineered disulfide bond between the Fab and the Fc, optionally wherein the engineered disulfide bond places the antigens or epitopes thereof in a more favorable orientation for divalent binding of at least one BCR and/or antibody. In some embodiments, the divalent immunogen comprises an engineered loop extension in the Fc, optionally wherein the engineered loop extension places the antigens or epitopes thereof in a more favorable orientation for divalent binding of at least one BCR and/or antibody.

In some embodiments, all or part of the antibody is humanized. In some embodiments, the Fc comprises amino acid substitutions corresponding to L234A/L235A/P329G in SEQ ID NO 1.

In some embodiments, the antigen is HIV gp120, or a fragment or variant. In some embodiments, the epitope is the CD4 binding site of HIV gp120. In some embodiments, the sequence of the HIV gp120 antigen comprises a sequence at least 80%, at least 85%, at least 90%, at least 92%, at least 95%, at least 98%, or at least 99%, identical to SEQ ID NO: 13. In some embodiments, the sequence of the HIV gp120 antigen comprises SEQ ID NO: 13. In some embodiments, the sequence of the HIV gp120 antigen comprises SEQ ID NO: 15.

In some embodiments, the first engineered antigen mutation of the HIV gp120 antigen, or fragment or variant thereof, corresponds to 1423C of HIV gp120. In some embodiments, at least one of the two antigens or epitopes thereof does not comprise the N276 glycan.

In some embodiments, the Fab domains are derived from a co-receptor binding site antibody. In some embodiments, the co-receptor binding site antibody is 48d. In some embodiments, the sequence of the Fab heavy chain comprises a sequence at least 80% identical to SEQ ID NO: 1. In some embodiments, the sequence of the Fab light chain comprises a sequence at least 80% identical to SEQ ID NO: 3.

In some embodiments, the first engineered Fab mutation corresponds to D56 of the 48d heavy chain. In some embodiments, the second engineered Fab mutation corresponds to K126C of the 48d light chain. In some embodiments, the second engineered Fab mutation comprises addition of at least one amino acid between the residues corresponding to amino acids 119 to 132 of the 48d light chain, optionally wherein the second engineered mutation additionally comprises substitution of at least one of the residues corresponding to amino acids 119 to 132 of the 48d light chain. In some embodiments, the sequence of the Fab light chain comprises any one of SEQ ID NOs: 5, 7, 9, or 11.

In some embodiments, the divalent immunogen provides precise positioning of two antigens or epitopes thereof such that certain BCRs are preferably bound to both antigens simultaneously.

In some embodiments, the two Fab domains of the bound BCR assume an angle of about 20 degrees to about 150 degrees. In some embodiments, the two Fab domains of the bound BCR assume an angle of about 90 degrees to about 140 degrees. In some embodiments, the two Fab domains of the bound BCR assume an angle of about 120 degrees.

In some embodiments, the divalent immunogen provides an antigenic spacing of about 3 to about 20 nm. In some embodiments, the divalent immunogen provides an antigenic spacing of about 7 to about 19 nm. In some embodiments, the divalent immunogen provides an antigenic spacing of about 13 to about 16 nm.

In some embodiments, the second engineered Fab mutation fixes the distance between the antigens or epitopes thereof.

In some embodiments, the second engineered Fab mutation modifies the interaction between the antigens or epitopes thereof and certain BCRs. In some embodiments, the second engineered Fab mutation enhances the binding avidity of certain BCRs and/or antibodies for the antigen or epitope thereof by at least 1.5-fold. In some embodiments, the second engineered Fab mutation enhances the binding avidity of certain BCRs and/or antibodies for the antigen or epitope thereof by at least 10-fold. In some embodiments, the second engineered Fab mutation enhances the binding avidity of certain BCRs and/or antibodies for the antigen or epitope thereof by at least 100-fold.

In some embodiments, the binding avidity of certain BCRs is at least 1.5-fold higher for the two antigens covalently linked to the scaffold compared to the same antigen unlinked to the scaffold. In some embodiments, the binding avidity of certain BCRs is at least 10-fold higher for the two antigens covalently linked to the scaffold compared to the same antigen unlinked to the scaffold. In some embodiments, the binding avidity of certain BCRs is at least 100-fold higher for the two antigens covalently linked to the scaffold compared to the same antigen unlinked to the scaffold.

In a further embodiment, the present disclosure provides a multimer of one or more of the divalent immunogens disclosed herein. In some embodiments, the monomers of the divalent immunogen are conjugated onto a nanoparticle. In some embodiments, the multimer is a dimer, a trimer, or a tetramer.

In a further embodiment, the present disclosure provides a pharmaceutical composition comprising any one or more of the divalent immunogens and/or multimers disclosed herein. In some embodiments, the pharmaceutical composition additionally comprises a pharmaceutically-acceptable solvent. In some embodiments, the pharmaceutical composition additionally comprises an adjuvant, optionally wherein the adjuvant is poly I:C.

In a further embodiment, the present disclosure provides polynucleotides encoding a scaffold and/or two antigens or epitopes thereof disclosed herein. In some embodiments, the polynucleotide encodes a scaffold or portion thereof, wherein the encoded scaffold provides precise positioning of two antigens or epitopes thereof such that only certain BCRs are preferably bound to both antigens simultaneously. In some embodiments, the polynucleotide encodes a Fab heavy chain disclosed herein. In some embodiments, the polynucleotide encodes a Fab light chain disclosed herein.

In a further embodiment, the present disclosure provides a cell comprising at least one polynucleotide disclosed herein.

In a further embodiment, the present disclosure provides a method of identifying antibodies with preferred characteristics for divalent immunogens, comprising: (a) providing a divalent immunogen comprising a known broadly neutralizing antibody and an antigen or epitope thereof; (b) administering the divalent immunogen to a subject, optionally a mouse; (c) isolating B cells from the subject; (d) screening the isolated B cells for ability to bind to the divalent immunogen in high avidity, bidentate fashion; and (e) sequencing the complementarity determining regions of B cells that bind as in (d).

In a further embodiment, the present disclosure provides a method of generating an immune response in a patient, comprising administering one or more divalent immunogens, multimers, and/or pharmaceutical compositions disclosed herein. In some embodiments, the immune response results in an increased number of B cells expressing a BCR that binds the divalent immunogen divalently with increased avidity. In some embodiments, the patient has previously been administered at least one dose of a vaccine, optionally wherein the dose was of a different vaccine. In some embodiments, the immune response results in an increased number of B cells expressing a VRC01-class BCR.

In a further embodiment, the present disclosure provides a method of forming a ring structure comprising a divalent immunogen disclosed herein and a BCR or antibody; wherein the divalent immunogen comprises a specific antigenic spacing that allows the BCR or antibody to bind with a Fab-Fab angle of about 20 degrees to about 150 degrees. In some embodiments, the Fab-Fab angle is about 90 degrees to about 140 degrees. In some embodiments, the Fab-Fab angle is about 120 degrees. In some embodiments, the ring structure is formed in a subject. In some embodiments, the subject is a human.

### FIGURE DESCRIPTIONS

FIG. 1A provides a schematic of an exemplary divalent immunogen comprising an antibody covalently linked to two copies of an HIV gp120 antigen. A disulfide bond crosslink (Fab-Fab disulfide) is shown between the two Fab domains, and between each Fab and a gp120 core. FIG. 1B provides a schematic of interaction of the divalent immunogen with an exemplary B-cell receptor (BCR), VRC01-class BCR. FIG. 1C provides a schematic of interaction of the VRC01-class BCR with a divalent immunogen comprising a loop extension mutation including an engineered disulfide.

FIG. 2 provides a detailed schematic of interaction of the divalent immunogen with an exemplary B-cell receptor (BCR), VRC01-class BCR. Fab-Fab linkage, engineered disulfide, gp120 and its CD4 binding site, and the divalent immunogen, are labeled.

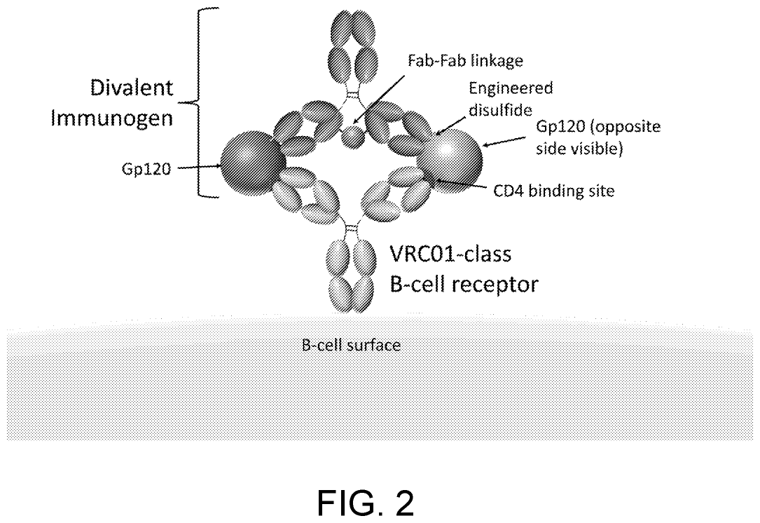

FIG. 3A provides a structural model of the interaction between an exemplary divalent immunogen (top) and an exemplary antibody, VRC01 (bottom). The VRC01 antibody is shown interacting with a divalent immunogen comprising two copies of the gp120 core and the 48d antibody. Disulfide bond formation between the 48d Fab and the gp120 core is enabled by a 1423C mutation in the gp120 core and a D56C mutation in the 48d Fab. The 48d Fab additionally comprises the K126C mutation (box). FIG. 3B provides an expanded view of the box in FIG. 2A. The structure of the light chain loop is shown for the 48d Fab with the K126C mutation, and for each of three loop extension mutations (Loop1, Loop2, and Loop3) which comprise a re-designed loop comprising a cysteine that enables Fab-Fab crosslinking. Loop extension mutations were predicted by Alphafold2. Sequences of the loop mutations, corresponding to amino acids 119 to 132 of the 48d light chain SEQ ID NO: 3 are shown, and cysteines shown in the model are underlined.

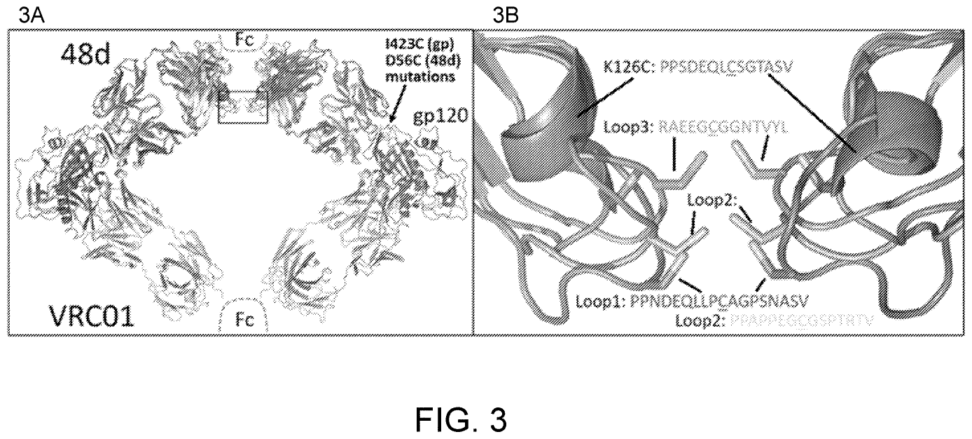

FIG. 4 provides an SDS-PAGE gel image showing divalent immunogens of the 48d antibody and gp120 antigen. Divalent immunogens were labeled at the Fc with a fluorophore-linked covalent DNA aptamer, allowing detection of the divalent immunogens electrophoresed through a native polyacrylamide gel, pulled by the negatively charged DNA. Lanes of the gel show divalent immunogens of the wild-type 48d antibody (48d IC), the 48d antibody with the K126C mutation (K126C IC), and the 48d antibody with each of the loop mutations (Loop1 IC, Loop 2 IC, and Loop 3 IC). Using a light chain with wtK126 or with substitution of the Loop1, Loop2, or Loop3 cysteine with an alanine in each of the loop mutant divalent immunogens (C→A) prevents crosslinking of the Fabs. 0, 1 and 2 refer to bands representing zero, one and two copies of gp120, respectively, bound to the 48d antibody. Arrows indicate the band suspected to be divalent immunogens of the 48d antibody with the K126C mutation bound to zero copies of gp120.

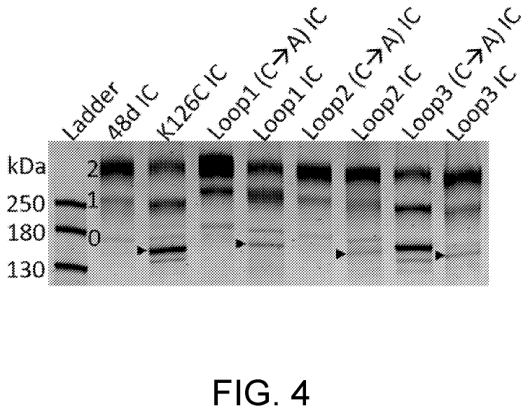

FIG. 5A provides a gel shift assay showing the effect of Fab crosslinking (via the K126C mutation) on binding of VRC01-class and non-VRC01-class antibodies to divalent immunogens of the 48d antibody and gp120 antigen. Cartoons at the left indicate the gp120 occupancy (0, 1 or 2) of the divalent immunogens, as shown in FIG. 3, for the adjacent band. The white arrow indicates the shift for Fab-crosslinked (K126C) divalent immunogens bound by two copies of the non-VRC01-class A32 antibody. Gray arrows indicate the shift for Fab-crosslinked or non-Fab-crosslinked (wtK126) divalent immunogens bound to one copy of either the A32 or wtVRC01 antibodies. FIG. 5B provides a gel shift assay showing the effect of the K126C mutation on binding of the 7mutVRC01 antibody to divalent immunogens of the 48d antibody and gp 120 antigen. The upper band in the lanes with K126C antibody indicates the shift for Fab-crosslinked divalent immunogens with double gp 120 occupancy bound by one copy of the 7mutVRC01 antibody.

FIG. 6 provides a graphical summary of gel shift assays performed with the 7mutVRC01 antibody and divalent immunogens described herein. Divalent immunogens comprised the wild-type 48d antibody (wtK126), the 48d antibody with the K126C mutation (K126C), and the 48d antibody with the loop extension mutations (Loop 1, Loop2, Loop3), and the 48d antibody with the loop extension mutations wherein the Loop1, Loop2, or Loop3 cysteine is substituted for an alanine (Loop1 (C→A), Loop2 (C→A), Loop3 (C→A)), covalently linked to gp120 antigen. Fraction Bound refers to the fraction of the divalent immunogen that is bound by the 7mutVRC01 antibody, as indicated by a shift in the band position on the gel. It is important to note that Fab-Fab-crosslinked samples also contain non-crosslinked species and therefore, curves are the summation of binding by both species.

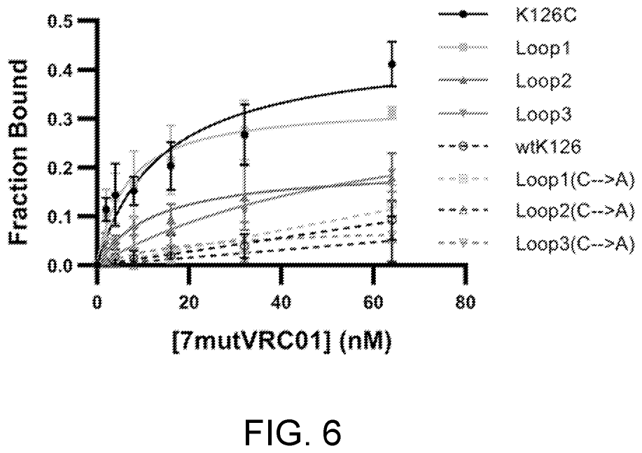

FIG. 7 provides a conjugation strategy for dimers and trimers of the divalent antigen. Two separate cultures-one expressing the 15.6 kDa SpyCatcher at the divalent scaffold interface (heavy chain C-termini for Fab-comprising divalent immunogens) and one expressing a 50/50 combination of SpyTagged and untagged divalent complex resulting in a mixed species containing zero (25%), one (50%) or two (25%) Spytags. Mixing of the two batches of divalent construct will result in dimers and trimers which can be separated by size exclusion chromatography (SEC).

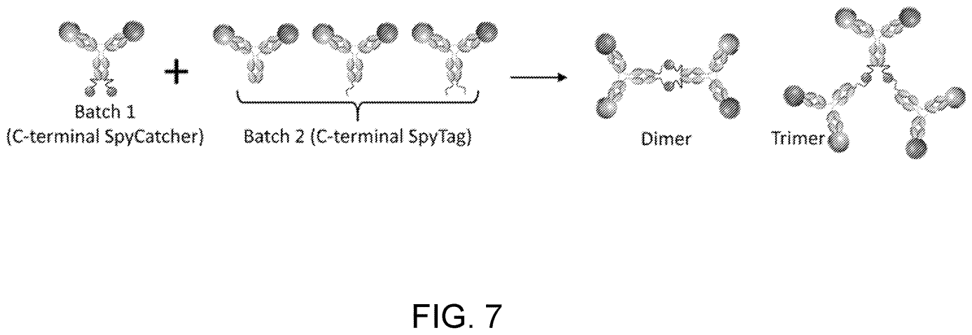

FIG. 8A provides a gel showing co-expression and purification of 48d-gp120 complexes in non-reducing conditions. Lane L) Ladder. Lane 1) supernatant after incubation of cell culture supernatant with protein A/G magnetic beads. Lane 2) Protein eluted from the beads with 48d containing 0, 1, and 2 copies of gp120. FIG. 8B provides a gel showing nanoparticle conjugation of 48d-gp120 divalent immunogens. Lane L) Ladder. Lane 1) Purified divalent immunogen from the left panel, non-reducing. Lane 2) Reduced material from lane 1. Lane 3) VLP-SpyCatcher protein. Lane 4) Reaction product after incubation of divalent immunogen-SpyTag with VLP-SpyCatcher. Lane 5) Purified gp120 core. HC-heavy chain, LC-light chain.

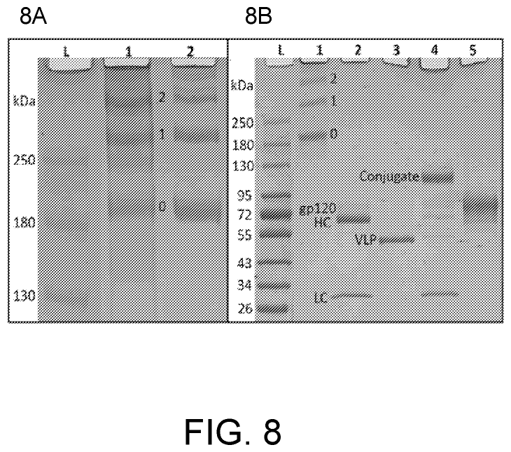

FIG. 9 provides an electron micrograph of nanoparticles decorated with divalent immunogens. The inset box is expanded in the bottom left-hand corner. A scale Pymol-generated model is shown next to the image for comparison.

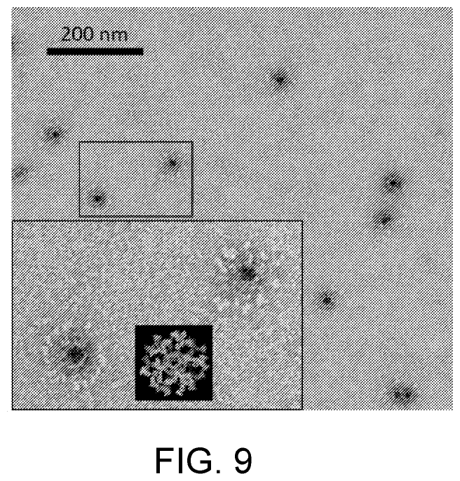

FIG. 10A provides an exemplary divalent immunogen in which covalent bonds between the Fabs and the Fc (e.g., disulfide bonds or chemical crosslinking) position the antigens optimally for divalent engagement by BCRs. FIG. 10B provides an exemplary divalent immunogen in which bulky loops are added to force the Fabs into a specific position.

FIG. 11A provides an exemplary divalent immunogen in which engineered loop extension or extension of the C-termini of the heavy and light chains create a stable Fab dimer interface. FIG. 11B provides an exemplary divalent immunogen in which a linker (e.g., peptide, DNA, or other organic material) fixes the antigens in an optimal orientation for divalent binding by targeted BCRs.

FIGS. 12A and 12B provide exemplary de novo protein-based scaffolds for divalent antigen/epitope/mimotope presentation.

FIGS. 13A and 13B provide exemplary DNA nanostructure-scaffolded divalent immunogens, with multiple anchor points (12A) or a single anchor point (12B) for each antigen.

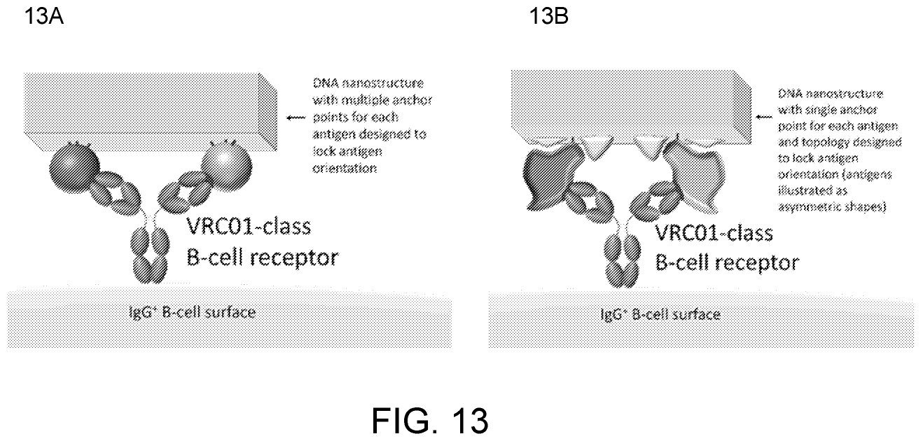

FIG. 14 provides a schematic of an exemplary AI-based design (RFD1) of a dimer interface for 48d Fabs.

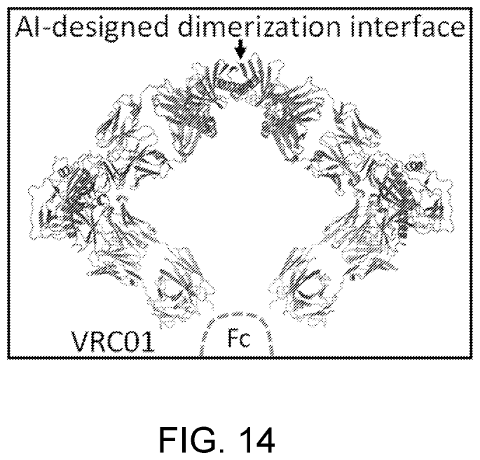

FIG. 15 provides a gel showing purified constructs of seven dimerization interfaces (RFD1-7) designed using RFDiffusion. The gel shows bands for the heavy chain conjugated to gp120, free heavy chain, and light chain.

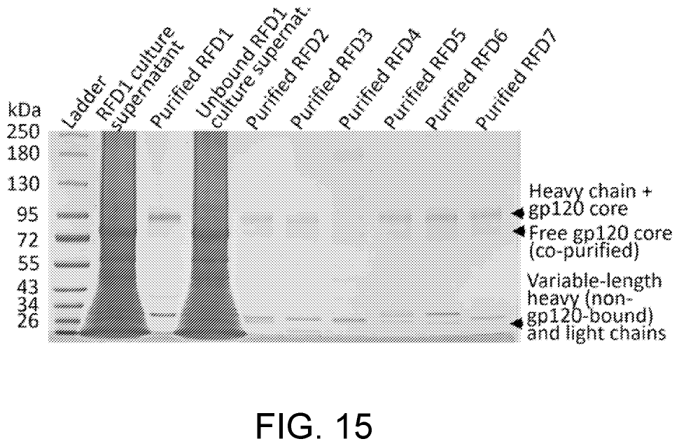

FIG. 16 shows a divalent immunogen design incorporating a scaffold based on dimeric protein 1gh7—the extracellular domain of the beta-common receptor of IL-3, IL-5 and GM-CSF. The design, which is a fusion of gp120 and 1gh7, positions the two copies of gp120 for engagement of both Fabs of the VRC01 bnAb simultaneously.

FIG. 17 shows divalent design incorporating Protein Data Bank structure 4 ATM (BAR domain of human Amphiphysin) as a dimeric scaffold for gp120 for divalent engagement with VRC01.

FIG. 18 provides a strategy for isolating complementary antibodies for VRC01. Gp120 is shown in darker gray. VRC01 antibody is shown above. The Fc of this antibody is modeled due to the lack of an intact structure in the protein databank. An intact bidentate complementary antibody is modeled and shown below.

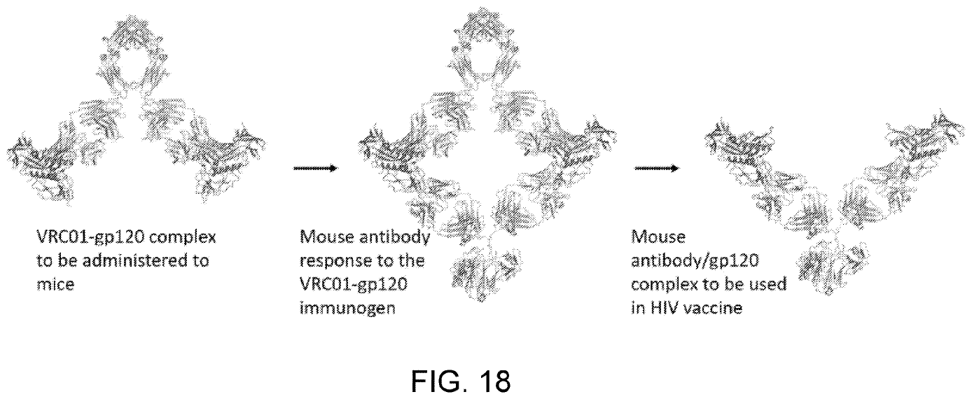

FIG. 19 provides a staining strategy to identify complementary antibody-producing B-cells. Intact and fragmented VRC01-gp120 complex is differentially labeled with fluorophores (spheres). Preferential binding of BCRs to intact VRC01-gp120 complex will indicate ideal binding geometry.

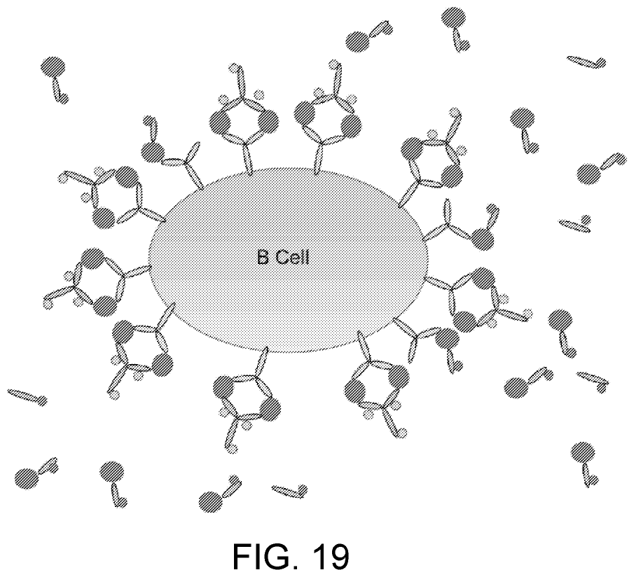

### DETAILED DESCRIPTION

### Overview

The present disclosure provides divalent immunogens comprising a scaffold and two antigens or epitopes thereof, wherein each antigen or epitope thereof is covalently linked to the scaffold. The present disclosure also provides multimers thereof, pharmaceutical compositions thereof, and polynucleotides encoding at least one antigen and/or epitope of the divalent immunogen, and/or the scaffold or a portion thereof. The present disclosure also provides methods of identifying antibodies for divalent immunogens described herein, and methods of use of divalent immunogens disclosed herein.

### Definitions

The following terms are used in the description herein and in the appended claims.

The singular forms “a,” “an” and “the” are intended to include the plural forms as well, unless the context clearly indicates otherwise.

The term “about” as used herein when referring to a measurable value such as an amount of the length of a polynucleotide or polypeptide, dose, time, temperature, and the like, is meant to encompass variations of ±20%, ±10%, ±5%, ±1%, ±0.5%, or even ±0.1% of the specified amount.

As used herein, “and/or” refers to and encompasses any and all possible combinations of one or more of the associated listed items, as well as the lack of combinations when interpreted in the alternative (“or”).

The terms “administering” or “introducing”, as used herein, refer to delivery of a composition to a cell, to cells, tissues and/or organs of a subject, or to a subject. Such administering or introducing may take place in vivo, in vitro or ex vivo.

The term “polynucleotide” as referred to herein means single-stranded or double-stranded nucleic acid polymers. In certain embodiments, the nucleotides comprising the polynucleotide can be ribonucleotides or deoxyribonucleotides or a modified form of either type of nucleotide. Said modifications include base modifications such as bromouridine, ribose modifications such as arabinoside and 2′,3′-dideoxyribose and internucleotide linkage modifications such as phosphorothioate, phosphorodithioate, phosphoroselenoate, phosphorodiselenoate, phosphoroanilothioate, phoshoraniladate and phosphoroamidate. The term “polynucleotide” specifically includes single and double stranded forms of DNA.

The terms “polypeptide,” “protein,” “peptide,” and “glycoprotein” are used interchangeably and mean a polymer of amino acids not limited to any particular length. The term does not exclude modifications such as myristylation, sulfation, glycosylation, phosphorylation and addition or deletion of signal sequences.

### Divalent Immunogens

The present disclosure provides divalent immunogens comprising a scaffold and two antigens or epitopes thereof. In some embodiments, each antigen or epitope thereof is covalently linked to the scaffold. As used herein, the term “immunogen” means a molecule or molecular assembly comprising at least one antigen or epitope. In some embodiments, the divalent immunogen comprises one polypeptide, two polypeptides, three polypeptides, four polypeptides, five polypeptides, six polypeptides, seven polypeptides, or eight polypeptides. Non-limiting applications for immunogens include administration to a subject to elicit an immune response.

As used herein, the term “scaffold” refers to a molecule or molecular assembly suitable for spatially orienting two antigens or epitopes thereof. In some embodiments, the scaffold comprises one polypeptide, two polypeptides, three polypeptides, four polypeptides, five polypeptides, six polypeptides, seven polypeptides, or eight polypeptides. In some embodiments, each antigen or epitope thereof is covalently linked to the scaffold. Covalent linkage may be via any method known in the art. Non-limiting examples of covalent linkage are disulfide bonds and peptide bonds.

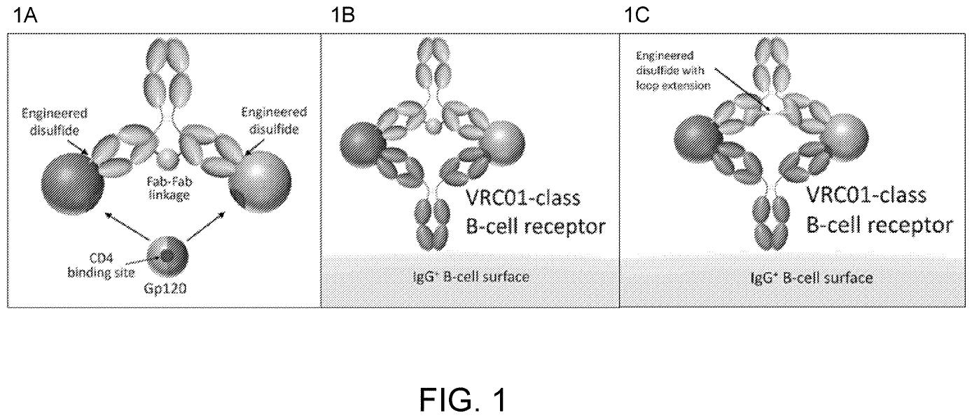

The divalent immunogens disclosed herein leverage the concept of avidity to enhance immune focusing (i.e., targeting specific epitopes for antibody response). The disclosed divalent immunogens provide the precise positioning of two antigens such that only certain B-cell receptors (BCRs), or the corresponding soluble antibodies, can bind to the antigens with both Fabs simultaneously, forming a ring dimer structure (shown in FIG. 1B). With significant rigidity of the divalent antigen and ideal binding geometry of targeted BCRs, the avidity effect is expected to be greatest. Optimal rigidity and binding geometry also minimize the entropic penalty for divalent binding—this is a significant factor especially when the monovalent affinity of the BCR (or antibody) is low.

### Divalent Immunogen Scaffolds

In some embodiments, the scaffold is derived from an antibody. In some embodiments, the scaffold comprises an antibody or a fragment or variant thereof.

As is well known in the art, an antibody is an immunoglobulin molecule capable of specific binding to a target, such as a carbohydrate, polynucleotide, lipid, polypeptide, etc., through at least one epitope recognition site, located in the variable region of the immunoglobulin molecule. Antibody fragments are encompassed by the present disclosure, such as Fab, F(ab′) 2, and Fv which include a heavy chain and light chain variable region and specifically bind a certain antigen. These antibody fragments retain the ability to selectively bind with the antigen.

These fragments include: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule can be obtained by treating whole antibody with pepsin, followed by reduction, to yield an intact light chain and a portion of the heavy chain; two Fab′ fragments are obtained per antibody molecule; (3) (Fab′)₂, the fragment of the antibody that can be obtained by treating whole antibody with the enzyme pepsin without subsequent reduction; F(ab′) 2 is a dimer of two Fab′ fragments held together by two disulfide bonds; (4) Fv, a genetically engineered fragment containing the variable region of the light chain and the variable region of the heavy chain expressed as two chains; and (5) Single chain antibody (such as scFv), defined as a genetically engineered molecule containing the variable region of the light chain, the variable region of the heavy chain, linked by a suitable polypeptide linker as a genetically fused single chain molecule. (6) A dimer of a single chain antibody (scFV₂), defined as a dimer of a scFV. This has also been termed a “miniantibody.” Methods of making these fragments are known in the art (see for example, Harlow and Lane, Antibodies: A Laboratory Manual, Cold Spring Harbor Laboratory, New York, 1988).

In some embodiments, an antibody or antigen-binding fragments thereof, e.g., a Fab, scFv, VHH or sdAb, may have: a) a heavy chain having an amino acid sequence that is at least 80%, at least 85%, at least 90%, at least 92%, at least 95%, at least 98%, or at least 99% identical to the heavy chain of an antibody or antigen-binding fragments thereof described herein; and/or b) a light chain having an amino acid sequence that is at least 80% identical, at least 85%, at least 90%, at least 95%, at least 98%, or at least 99% identical to the light chain of an antibody or antigen-binding fragments thereof described herein. The amino acid sequences of illustrative antibodies and fragments thereof are set forth herein.

The antibody from which the scaffold is derived may be from any known class. Non-limiting examples include IgA (including subclasses IgAQ1 and IgA2), IgD, IgE, IgG (including subclasses IgG1, IgG2, IgG3, and IgG4), and IgM.

In some embodiments, the scaffold comprises two Fab fragments or variants thereof. In some embodiments, each antigen or epitope is covalently linked to a Fab fragment. Non-limiting examples of covalent linkers are disulfide bonds, peptide bonds, chemical crosslinkers, protein crosslinkers, and nucleic acid crosslinkers.

In some embodiments, the Fab fragments are derived from a co-receptor binding site antibody. In some embodiments, the Fab fragments comprise a heavy chain sequence at least 80%, at least 85%, at least 90%, at least 92%, at least 95%, at least 98%, or at least 99% identical to the heavy chain sequence of a co-receptor antibody. In some embodiments, the Fab fragments comprise a light chain sequence at least 80%, at least 85%, at least 90%, at least 92%, at least 95%, at least 98%, or at least 99% identical to the light chain sequence of a co-receptor antibody.

In some embodiments, the co-receptor binding site antibody is 48d. In some embodiments, the Fab fragments comprise a heavy chain sequence at least 80%, at least 85%, at least 90%, at least 92%, at least 95%, at least 98%, or at least 99% identical to the heavy chain sequence of the 48d antibody. In some embodiments, the Fab fragments comprise a light chain sequence at least at least 80%, at least 85%, at least 90%, at least 92%, at least 95%, at least 98%, or at least 99% identical to the light chain sequence of the 48d antibody. In some embodiments, the Fab fragments comprise a heavy chain sequence at least 80%, at least 85%, at least 90%, at least 92%, at least 95%, at least 98%, or at least 99% identical to the heavy chain sequence of SEQ ID NO: 1. In some embodiments, the Fab fragments comprise a light chain sequence at least 80%, at least 85%, at least 90%, at least 92%, at least 95%, at least 98%, or at least 99% identical to the light chain sequence of SEQ ID NO: 3. In some embodiments, the Fab fragments comprise a heavy chain sequence comprising SEQ ID NO: 1. In some embodiments, the Fab fragments comprise a light chain sequence comprising SEQ ID NO: 3. In some embodiments, the Fab fragments comprise a heavy chain sequence comprising SEQ ID NO: 1 and a light chain sequence comprising SEQ ID NO: 3.

In some embodiments, the Fab fragments comprise a light chain sequence comprising SEQ ID NO: 5. In some embodiments, the Fab fragments comprise a light chain sequence comprising any one of SEQ ID NOs: 7, 9, and 11. In some embodiments, the Fab fragments comprise a heavy chain sequence comprising SEQ ID NO: 1 and a light chain sequence comprising SEQ ID NO: 5. In some embodiments, the Fab fragments comprise a heavy chain sequence comprising SEQ ID NO: 1 and a light chain sequence comprising SEQ ID NO: 7. In some embodiments, the Fab fragments comprise a heavy chain sequence comprising SEQ ID NO: 1 and a light chain sequence comprising SEQ ID NO: 9. In some embodiments, the Fab fragments comprise a heavy chain sequence comprising SEQ ID NO: 1 and a light chain sequence comprising SEQ ID NO: 11.

In some embodiments, each Fab fragment comprises at least a first engineered Fab mutation. In some embodiments, the first engineered Fab mutation enables formation of the disulfide bond between the Fab fragment and the antigen or epitope thereof. Non-limiting examples of engineered mutations include addition, deletion, substitution, or insertion of one or more amino acids into the amino acid sequence of the Fab fragment. In some embodiments, the mutation comprises addition, deletion, substitution, or insertion of one or more amino acids into the amino acid sequence of the Fab heavy chain sequence. In other embodiments, the mutation comprises addition, deletion, substitution, or insertion of one or more amino acids into the amino acid sequence of the Fab light chain sequence. In some embodiments, the first engineered Fab mutation comprises a substitution corresponding to D56C of SEQ ID NO: 1.

In some embodiments, each Fab fragment comprises at least a second engineered Fab mutation that enables Fab-Fab crosslinking. In some embodiments, the second engineered Fab mutation is in the light chain of each of the two Fab fragments. In some embodiments, the second engineered Fab mutation is in the heavy chain of each of the two Fab fragments. In some embodiments, the mutation comprises addition, deletion, substitution, or insertion of one or more amino acids into the amino acid sequence of the Fab heavy chain sequence. In other embodiments, the mutation comprises addition, deletion, substitution, or insertion of one or more amino acids into the amino acid sequence of the Fab light chain sequence. In some embodiments, the second engineered Fab mutation comprises a substitution corresponding to K126C of SEQ ID NO: 3.

In some embodiments, the second engineered Fab mutation places the antigens or epitopes thereof in a more favorable orientation for divalent binding of at least one antibody. In some embodiments, the second engineered Fab mutation comprises addition of at least one amino acid between the residues corresponding to amino acids 119 to 132 of SEQ ID NO: 3. In some embodiments, the second engineered mutation additionally comprises substitution of at least one of the residues corresponding to amino acids 119 to 132 of SEQ ID NO: 3.

In some embodiments wherein the scaffold comprises a Fab domain, the divalent immunogen comprising the Fab additionally comprises an Fc domain. The Fc region may be derived from any of a variety of different Fcs, including but not limited to, a wild-type or modified IgG1, IgG2, IgG3, IgG4 or other isotype, e.g., wild-type or modified human or murine IgG1, human or murine IgG2, human or murine IgG3, human IgG4, human IgG4Pro (comprising a mutation in core hinge region that prevents the formation of IgG4 half molecules), human or murine IgA, human or murine IgE, human or murine IgM, or human or murine IgM. In some embodiments, the Fc domain is derived from a human IgG1. In some embodiments, the Fc domain is derived from a murine IgG2a. In some embodiments, all or part of the Fc is humanized.

A “humanized” immunoglobulin is an immunoglobulin including a human framework region and one or more CDRs from a non-human (such as a mouse, rat, or synthetic) immunoglobulin. The non-human immunoglobulin providing the CDRs is termed a “donor,” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, such as at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and/or a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. Humanized immunoglobulins can be constructed by means of genetic engineering (for example, see U.S. Pat. No. 5,585,089).

In some embodiments, the divalent immunogen comprises an engineered dimerization interface comprising C-terminal extension of the Fab heavy and/or light chains. In some embodiments, the divalent immunogen comprises an engineered dimerization interface comprising C-terminal extension of the Fc heavy and/or light chains. The C-terminal extension may comprise addition or insertion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or least 20 amino acids to the Fab and/or Fc heavy and/or light chains. In some embodiments, the engineered dimerization interface places the antigens or epitopes thereof in a more favorable orientation for divalent binding of at least one BCR and/or antibody.

In some embodiments, the Fc comprises amino acid substitutions corresponding to L234A, L235A, P329G (LALA-PG) in SEQ ID NO: 1 and/or as described in Lo et al. J. Biol. Chem. 2017; 292, p. 3900-3908. The LALA-PG variant has been shown to eliminate complement binding and fixation as well as Fc-g dependent antibody-dependent cell-mediated cytotoxicity (ADCC) in both murine IgG2a and human IgG1. These LALA-PG substitutions allow a more accurate translation of results generated with an “effectorless” antibody framework scaffold between mice and primates.

The present disclosure also provides divalent immunogens wherein the divalent immunogen comprises an engineered disulfide bond between the Fab and the Fc, optionally wherein the engineered disulfide bond places the antigens or epitopes thereof in a more favorable orientation for divalent binding of at least one antibody.

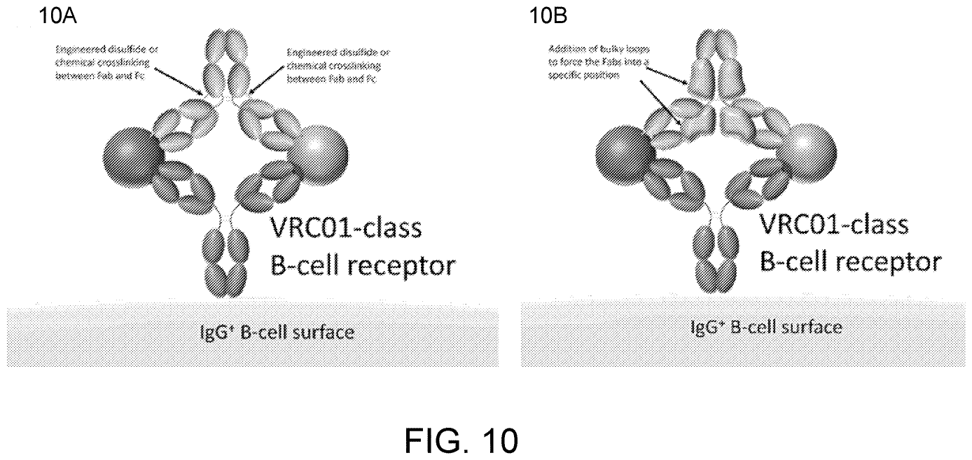

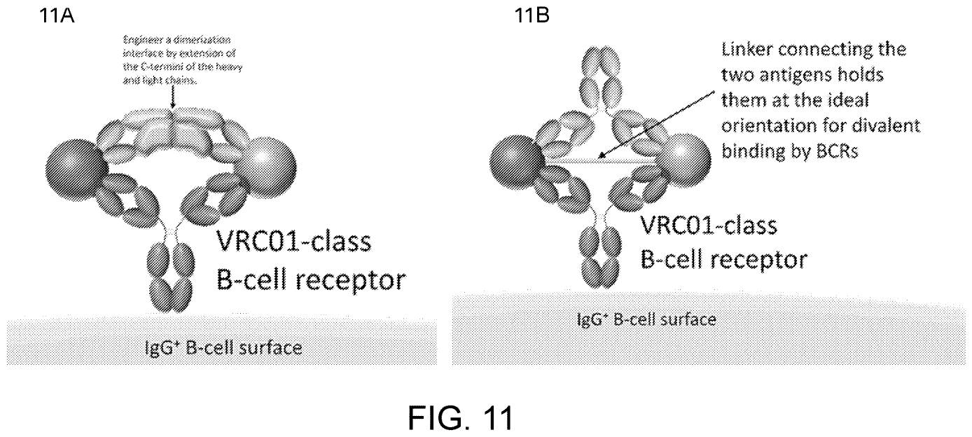

Divalent immunogens that position the antigens ideally for engagement by BCRs may also be designed by creating a covalent bond between the Fabs and the Fc (e.g., an engineered disulfide or chemical crosslinking, as in FIG. 10A). Disulfide formation could be accomplished by, for example, point mutations within Fab and Fc to cysteines, and/or loop extension in the Fab and/or Fc and incorporation of cysteines. Chemical crosslinking could be accomplished by e.g. cysteine point mutation/dimaleimide crosslinking. Divalent immunogens that position the antigens ideally for engagement by BCRs may also be designed by engineering a rigid antibody structure by adding bulky loop extensions (e.g., as in FIG. 10B), by adding a loop extension or extension of the C-termini of the heavy and/or light chains to come together and form a stable Fab dimer interface (e.g., as in FIG. 11A), or by adding a linker connecting the two antigens that holds them at the ideal orientation (e.g., as in FIG. 11B).

In some embodiments, the divalent immunogen comprises a linker between the two antigens or epitopes thereof. Non-limiting examples of the linker are a peptide linker, a nucleic acid linker, concatamerization of the antigens in a single gene, connection by chemical crosslinking, and connection using exogenous proteins. In some embodiments, the linker places the antigens or epitopes thereof in a more favorable orientation for divalent binding of at least one BCR and/or antibody.

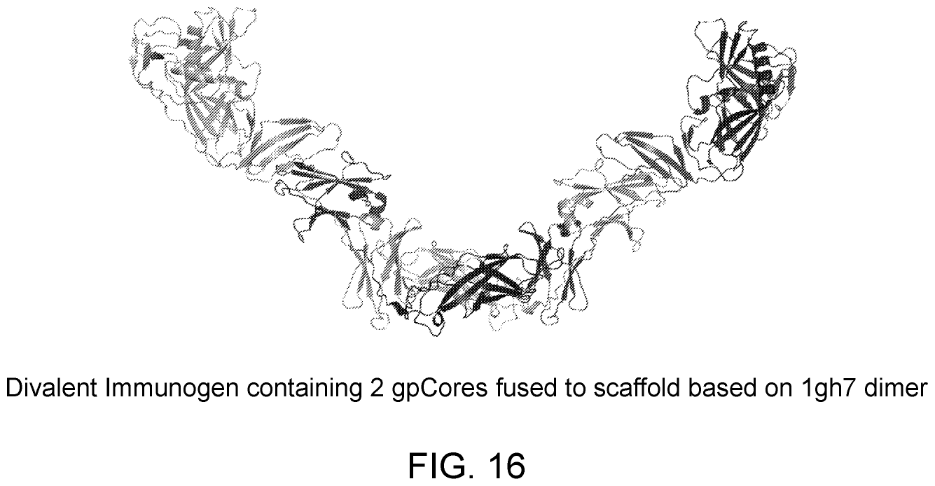

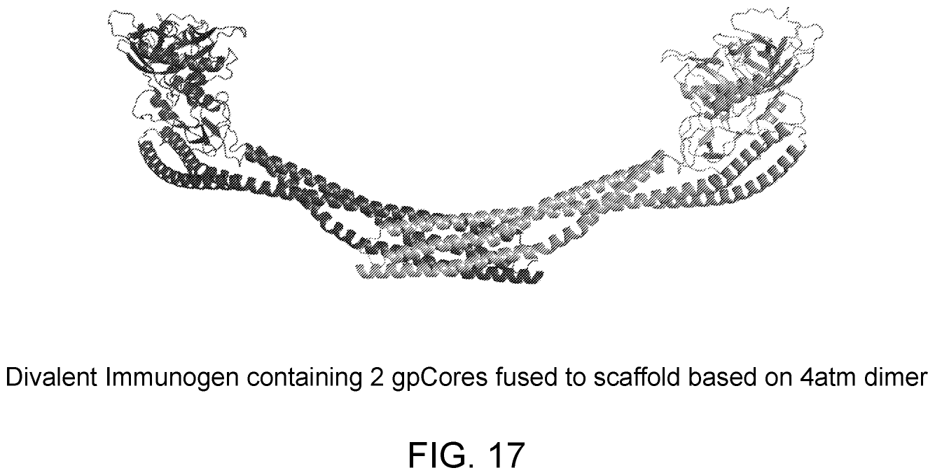

In some embodiments, the scaffold is derived from a dimeric protein. In other embodiments, the scaffold is derived from a multimeric protein. In still other embodiments, the scaffold is derived from a monomeric protein. Non-limiting examples of divalent immunogens incorporating a scaffold based on a dimeric or multimeric protein are shown in FIGS. 16 and 17, and in SEQ ID NOs: 71 and 72.

In some embodiments, the scaffold comprises a partially de novo engineered protein scaffold. Scaffolds comprising a partially de novo engineered protein scaffold may be based on a monomeric, dimeric, or multimeric protein.

In other embodiments, the scaffold comprises a completely de novo engineered protein scaffold. Scaffolds comprising a completely de novo engineered protein scaffold may be based on a monomeric, dimeric, or multimeric protein.

In other embodiments, the scaffold comprises a nucleic acid nanostructure. Non-limiting examples of a nucleic acid nanostructure include a DNA nanostructure (Seeman and Sleiman. Nat Rev Mater. 2018. 3, 17068).

Non-limiting examples of sequences that such a divalent immunogen comprising a scaffold the comprises a partially or fully de novo engineered protein scaffold include SEQ ID NO: 19, 21, 23, 25, 27, 29, 31, 33, 35, 37, 39, 41, 43, 45, 47, 63, 65, 67, 69, and 71.

In other embodiments, the divalent immunogen comprises a fusion protein of an antibody light chain (e.g., the 48d antibody light chain) and one antigen (e.g., gp120) expressed as a single polypeptide. In this embodiment, the C-terminus of the antigen is fused with the N-terminus of the light chain. The non-fused heavy chain is co-expressed. Two copies of the fusion protein and two copies of the non-fused heavy chain together form a divalent immunogen. A non-limiting example of a sequence that such a divalent immunogen comprising a fusion protein may comprise is SEQ ID NO: 61.

In other embodiments, the divalent immunogen comprises a fusion protein of an antibody heavy chain (e.g., the 48d antibody heavy chain) and one antigen (e.g., gp120) expressed as a single polypeptide. In this embodiment, the C-terminus of the antigen is fused with the N-terminus of the heavy chain. The non-fused light chain is co-expressed. Two copies of the fusion protein and two copies of the non-fused light chain together form a divalent immunogen. A non-limiting example of a sequence that such a divalent immunogen comprising a fusion protein may comprise is SEQ ID NO: 17.

Non-limiting examples of sequences that the divalent immunogen scaffold may comprise are shown in Table 1 and SEQ ID NOs: 1, 3, 5, 7, 9, 11, 17, 19, 21, 23, 25, 27, 29, 31, 33, 35, 37, 39, 41, 43, 45, 47, 49, 51, 53, 61, 63, 65, 67, 69, and 71.

TABLE 1
Non-limiting examples of polypeptides comprised by divalent immunogen
scaffolds

| | SEQ | |
|---|---|---|
| | ID | |
| ID | NO. | Sequence |
| 48d | 1 | MGWSCIILFLVATATGVHSEVQLVQSGAEVKKPGATVKISCKASGYTFSDFYMY |
| antibody | | WVRQAPGKGLEWMGLIDPEDACTMYAEKFRGRVTITADTSTDTGYLELSSLRSE |
| HC | | DTAVYYCAADPWELNAFNVWGQGTLVSVSSASTKGPSVFPLAPSSKSTSGGTAA |
| | | LGCLVKDYFPEPVTVSWNSGALTSGVHTFPAVLQSSGLYSLSSVVTVPSSSLGTQT |
| | | YICNVNHKPSNTKVDKKVEPKSCDKTHTCPPCPAPNAAGGPSVFIFPPKIKDVLMI |
| | | SLSPIVTCVVVDVSEDDPDVQISWFVNNVEVHTAQTQTHREDYNSTLRVVSALPI |
| | | QHQDWMSGKEFKCKVNNKDLGAPIERTISKPKGSVRAPQVYVLPPPEEEMTKKQ |
| | | VTLTCMVTDFMPEDIYVEWTNNGKTELNYKNTEPVLDSDGSYFMYSKLRVEKKN |
| | | WVERNSYSCSVVHEGLHNHHTTKSFSRTPG |
| 48d | 3 | MGWSCIILFLVATATGVHSDIQMTQSPSSVSASVGDRVTITCRASQDISTWLAWY |
| antibody LC | | QQKPGKAPKLLIYAASTLQSGVPSRFSGSGSGTDFSLTINSLQPEDFATYYCQQAN |
| | | SFFTFGGGTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVTHQGLSSP |
| | | VTKSFNRGEC |
| 48d | 5 | MGWSCIILFLVATATGVHSDIQMTQSPSSVSASVGDRVTITCRASQDISTWLAWY |
| antibody LC | | QQKPGKAPKLLIYAASTLQSGVPSRFSGSGSGTDFSLTINSLQPEDFATYYCQQAN |
| K126C | | SFFTFGGGTKVEIKRTVAAPSVFIFPPSDEQLCSGTASVVCLLNNFYPREAKVQWK |
| | | VDNALQSGNSQESVTEQDSKDSTYSLSSTLTLSKADYEKHKVYACEVTHQGLSSP |
| | | VTKSFNRGEC |
| 48d | 7 | MGWSCIILFLVATATGVHSDIQMTQSPSSVSASVGDRVTITCRASQDISTWLAWY |
| antibody LC | | QQKPGKAPKLLIYAASTLQSGVPSRFSGSGSGTDFSLTINSLQPEDFATYYCQQAN |
| Loop1 | | SFFTFGGGTKVEIKRTVAAPSVFIFPPNDEQLLPCAGPSNASVVCLLNNFYPREAKV |
| | | QWKVDNALQSGNSQESVTEQDSKDSTYSLSSTLTLSKADYEKHKVYACEVTHQG |
| | | LSSPVTKSFNRGEC |
| 48d | 9 | MGWSCIILFLVATATGVHSDIQMTQSPSSVSASVGDRVTITCRASQDISTWLAWY |
| antibody LC | | QQKPGKAPKLLIYAASTLQSGVPSRFSGSGSGTDFSLTINSLQPEDFATYYCQQAN |
| Loop2 | | SFFTFGGGTKVEIKRTVAAPSVFIFPPAPPEGCGSPTRTVVCLLNNFYPREAKVQW |
| | | KVDNALQSGNSQESVTEQDSKDSTYSLSSTLTLSKADYEKHKVYACEVTHQGLSS |
| | | PVTKSFNRGEC |
| 48d | 11 | MGWSCIILFLVATATGVHSDIQMTQSPSSVSASVGDRVTITCRASQDISTWLAWY |
| antibody LC | | QQKPGKAPKLLIYAASTLQSGVPSRFSGSGSGTDFSLTINSLQPEDFATYYCQQAN |
| Loop3 | | SFFTFGGGTKVEIKRTVAAPSVFIFRAEEGCGGNTVYLVCLLNNFYPREAKVQWK |
| | | VDNALQSGNSQESVTEQDSKDSTYSLSSTLTLSKADYEKHKVYACEVTHQGLSSP |
| | | VTKSFNRGEC |
| gp120.Core. | 17 | MPMGSLQPLATLYLLGMLVASVLADIRQAHCNVSKATWNETLGKVVKQLRKHF |
| 48D + 48D. | | GNNTIIRFANSSGGDLEVTTHSFNCGGEFFYCNTSGLFNSTWISNTSVQGSNSTGSN |
| Heavy_ | | DSITLPCRIKQCINMWQRIGQAMYAPPIQGVIRCVSNITGLILTRDGGSTNSTTETFR |
| Fusion | | PGGGDMRDNWRSELYKYKVVKIEGGVWKDAETTLFCASDAKAYETEKHNVWA |
| | | THACVPTDPNPQEIHLEGVTEEFNMWKNNMVEQMHTDIISLWDQSLKPCVKLTG |
| | | GSAITQACPKVSFEPIPIHYCAPAGFAILKCKDKKFNGTGPCPSVSTVQCTHGIKPV |
| | | VSTQLLLNGSLAEEEVMIRSENITNNAKNILVQFNTPVQINCTRPGNGGGGGSGGG |
| | | SGGGSEVQLVQSGAEVKKPGATVKISCKASGYTFSDFYMYWVRQAPGKGLEWM |
| | | GLIDPEDACTMYAEKFRGRVTITADTSTDTGYLELSSLRSEDTAVYYCAADPWEL |
| | | NAFNVWGQGTLVSVSSASTKGPSVFPLAPSSKSTSGGTAALGCLVKDYFPEPVTV |
| | | SWNSGALTSGVHTFPAVLQSSGLYSLSSVVTVPSSSLGTQTYICNVNHKPSNTKVD |
| | | KKVEPKSCDKTHTCPPCPAPNAAGGPSVFIFPPKIKDVLMISLSPIVTCVVVDVSED |
| | | DPDVQISWFVNNVEVHTAQTQTHREDYNSTLRVVSALPIQHQDWMSGKEFKCKV |
| | | NNKDLGAPIERTISKPKGSVRAPQVYVIPPPEEEMTKKQVTLTCMVTDFMPEDIY |
| | | VEWTNNGKTELNYKNTEPVLDSDGSYFMYSKLRVEKKNWVERNSYSCSVVHEG |
| | | LHNHHTTKSFSRTPG |
| RFD1_Heavy | 19 | MGWSCIILFLVATATGVHSEVQLVQSGAEVKKPGATVKISCKASGYTFSDFYMY |
| | | WVRQAPGKGLEWMGLIDPEDACTMYAEKFRGRVTITADTSTDTGYLELSSLRSE |
| | | DTAVYYCAADPWELNAFNVWGQGTLVSVSSASTKGPSVFPLAPSSKSTSGGTAA |
| | | LGCLVKDYFPEPVTVSWNSGALTSGVHTFPAVLQSSGLYSLSSVVTVPSSSLGTQT |
| | | YICNVNHKPSNTKVDKKVTANTEVTPESVRAALRELFGDEELLAWAAELLRA |
| RFD1_Light | 21 | MGWSCIILFLVATATGVHSDIQMTQSPSSVSASVGDRVTITCRASQDISTWLAWY |
| | | QQKPGKAPKLLIYAASTLQSGVPSRFSGSGSGTDFSLTINSLQPEDFATYYCQQAN |
| | | SFFTFGGGTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVTHQGLSSP |
| | | VTVSYEKLGPGLWIYQTIQDGKLLSISTNAKPSTPLEEKVRAVAIREKLIAENAA |
| RFD1_Light. | 23 | MPMGSLQPLATLYLLGMLVASVLASAITQACPKVSFEPIPIHYCAPAGFAILKCKD |
| gpFusion: | | KKFNGTGPCPSVSTVQCTHGIKPVVSTQLLLNGSLAEEEVMIRSENITNNAKNILV |
| | | QFNTPVQINCTRPGNGGDIRQAHCNVSKATWNETLGKVVKQLRKHFGNNTIIRFA |
| | | NSSGGDLEVTTHSFNCGGEFFYCNTSGLFNSTWISNTSVQGSNSTGSNDSITLPCRI |
| | | KQCINMWQRIGQAMYAPPIQGVIRCVSNITGLILTRDGGSTNSTTETFRPGGGDMR |
| | | DNWRSELYKYKVVKIEGGVWKDAETTLFCASDAKAYETEKHNVWATHACVPTD |
| | | PNPQEIHLEGVTEEFNMWKNNMVEQMHTDIISLWDQSLKPCVKLTGGGSGGGSG |
| | | GGSDIQMTQSPSSVSASVGDRVTITCRASQDISTWLAWYQQKPGKAPKLLIYAAS |
| | | TLQSGVPSRFSGSGSGTDFSLTINSLQPEDFATYYCQQANSFFTFGGGTKVEIKRTV |
| | | AAPSVFIFPPSDEQLKSGTASVVCLLNNFYPREAKVQWKVDNALQSGNSQESVTE |
| | | QDSKDSTYSLSSTLTLSKADYEKHKVYACEVTHQGLSSPVTVSYEKLGPGLWIYQ |
| | | TIQDGKLLSISTNAKPSTPLEEKVRAVAIREKLIAENAA |
| RFD2_Heavy | 25 | MGWSCIILFLVATATGVHSEVQLVQSGAEVKKPGATVKISCKASGYTFSDFYMY |
| | | WVRQAPGKGLEWMGLIDPEDACTMYAEKFRGRVTITADTSTDTGYLELSSLRSE |
| | | DTAVYYCAADPWELNAFNVWGQGTLVSVSSASTKGPSVFPLAPSSKSTSGGTAA |
| | | LGCLVKDYFPEPVTVSWNSGALTSGVHTFPAVLQSSGLYSLSSVVTVPSSSLGTQT |
| | | YICNVNHKPSNTKVDKKVSTPVEAVLPPEALAHLRAFLALLPLEAALAVAHAVRQ |
| | | ALIAVQ |
| RFD2_Light | 27 | MGWSCIILFLVATATGVHSDIQMTQSPSSVSASVGDRVTITCRASQDISTWLAWY |
| | | QQKPGKAPKLLIYAASTLQSGVPSRFSGSGSGTDFSLTINSLQPEDFATYYCQQAN |
| | | SFFTFGGGTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVTHQGLSSP |
| | | VTARILRVNDVVFTSTQTETSGEWTVNSTPLDQLLLDIYLEAQAA |
| RFD3_Heavy | 29 | MGWSCIILFLVATATGVHSEVQLVQSGAEVKKPGATVKISCKASGYTFSDFYMY |
| | | WVRQAPGKGLEWMGLIDPEDACTMYAEKFRGRVTITADTSTDTGYLELSSLRSE |
| | | DTAVYYCAADPWELNAFNVWGQGTLVSVSSASTKGPSVFPLAPSSKSTSGGTAA |
| | | LGCLVKDYFPEPVTVSWNSGALTSGVHTFPAVLQSSGLYSLSSVVTVPSSSLGTQT |
| | | YICNVNHKPSNTKVDKKVEPKNSVTLTSTAADRELEEALRKLAPEAKFVLVGTLP |
| | | DGSTLVIVFVS |
| RFD3_Light | 31 | MGWSCIILFLVATATGVHSDIQMTQSPSSVSASVGDRVTITCRASQDISTWLAWY |
| | | QQKPGKAPKLLIYAASTLQSGVPSRFSGSGSGTDFSLTINSLQPEDFATYYCQQAN |
| | | SFFTFGGGTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVTHQGLSSP |
| | | VTKSFNKYPGVVVTETVDEATGTTTITLNTKNYKAVRLAIKIAEALHKLRPGSTTV |
| | | AVIS |
| RFD4_Heavy | 33 | MGWSCIILFLVATATGVHSEVQLVQSGAEVKKPGATVKISCKASGYTFSDFYMY |
| | | WVRQAPGKGLEWMGLIDPEDACTMYAEKFRGRVTITADTSTDTGYLELSSLRSE |
| | | DTAVYYCAADPWELNAFNVWGQGTLVSVSSASTKGPSVFPLAPSSKSTSGGTAA |
| | | LGCLVKDYFPEPVTVSWNSGALTSGVIITFPAVLQSSGLYSLSSVVTVPSSSLGTQT |
| | | YICNVNHKPSNTKVDKKVPTNKVVTVTIEVKEEELPEDVSVRIRMEIDISEKTGKV |
| | | EMRIEVSKS |
| RFD4_Light | 35 | MGWSCIILFLVATATGVHSDIQMTQSPSSVSASVGDRVTITCRASQDISTWLAWY |
| | | QQKPGKAPKLLIYAASTLQSGVPSRFSGSGSGTDFSLTINSLQPEDFATYYCQQAN |
| | | SFFTFGGGTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVTHQGLSSP |
| | | VTVTIHRFKPNNVVTIEKLPSGKIKVTLTAENVEEALVNAYRLALELAEE |
| RFD5_Heavy | 37 | MGWSCIILFLVATATGVHSEVQLVQSGAEVKKPGATVKISCKASGYTFSDFYMY |
| | | WVRQAPGKGLEWMGLIDPEDACTMYAEKFRGRVTITADTSTDTGYLELSSLRSE |
| | | DTAVYYCAADPWELNAFNVWGQGTLVSVSSASTKGPSVFPLAPSSKSTSGGTAA |
| | | LGCLVKDYFPEPVTVSWNSGALTSGVHTFPAVLQSSGLYSLSSVVTVPSSSLGTQT |
| | | YICNVNHKPSNTKVDKKVEPKNWVKATVKVEDGVVEAEFDSGGSKNIIEGLANS |
| | | MKLLAEVALELLS |
| RFD5_Light | 39 | MGWSCIILFLVATATGVHSDIQMTQSPSSVSASVGDRVTITCRASQDISTWLAWY |
| | | QQKPGKAPKLLIYAASTLQSGVPSRFSGSGSGTDFSLTINSLQPEDFATYYCQQAN |
| | | SFFTFGGGTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVTHQGLSSP |
| | | VTKSFNRLSSGTWATIEKKEDPGEMNVTANVANVISAKIKVEFK |
| RFD6_Heavy | 41 | MGWSCIILFLVATATGVHSEVQLVQSGAEVKKPGATVKISCKASGYTFSDFYMY |
| | | WVRQAPGKGLEWMGLIDPEDACTMYAEKFRGRVTITADTSTDTGYLELSSLRSE |
| | | DTAVYYCAADPWELNAFNVWGQGTLVSVSSASTKGPSVFPLAPSSKSTSGGTAA |
| | | LGCLVKDYFPEPVTVSWNSGALTSGVHTFPAVLQSSGLYSLSSVVTVPSSSLGTQT |
| | | YICNVNHKPSNTKVDKKVEPKEVLNRVTVKVPAELTEAEIAMLKTLLKALGEAIA |
| | | KAAK |
| RFD6_Light | 43 | MGWSCIILFLVATATGVHSDIQMTQSPSSVSASVGDRVTITCRASQDISTWLAWY |
| | | QQKPGKAPKLLIYAASTLQSGVPSRFSGSGSGTDFSLTINSLQPEDFATYYCQQAN |
| | | SFFTFGGGTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVTHQGLSSP |
| | | VTKSFNKETGLETDVDPKDADTPEYKRVTVYTPLTAEDVELFKKLAEKLAEAIYE |
| | | AAKER |
| RFD7_Heavy | 45 | MGWSCIILFLVATATGVHSEVQLVQSGAEVKKPGATVKISCKASGYTFSDFYMY |
| | | WVRQAPGKGLEWMGLIDPEDACTMYAEKFRGRVTITADTSTDTGYLELSSLRSE |
| | | DTAVYYCAADPWELNAFNVWGQGTLVSVSSASTKGPSVFPLAPSSKSTSGGTAA |
| | | LGCLVKDYFPEPVTVSWNSGALTSGVHTFPAVLQSSGLYSLSSVVTVPSSSLGTQT |
| | | YICNVNHKPSNTKVDKKVEPKNGFSLSGSPEEVVAQALKHHTGDEKVAKMGVAL |
| | | VKELLA |
| RFD7_Light | 47 | MGWSCIILFLVATATGVHSDIQMTQSPSSVSASVGDRVTITCRASQDISTWLAWY |
| | | QQKPGKAPKLLIYAASTLQSGVPSRFSGSGSGTDFSLTINSLQPEDFATYYCQQAN |
| | | SFFTFGGGTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVTHQGLSSP |
| | | VTKSFNRYPSGVIVYRTTSNGSSSYGIDANLKTLEALLEFIEAVVEIYKER |
| 48D_H.C56D. | 49 | MGWSCIILFLVATATGVHSEVQLVQSGAEVKKPGATVKISCKASGYTFSDFYMY |
| MouseIgG2a. | | WVRQAPGKGLEWMGLIDPEDACTMYAEKFRGRVTITADTSTDTGYLELSSLRSE |
| Kdel. | | DTAVYYCAADPWELNAFNVWGQGTLVSVSSASTKGPSVFPLAPSSKSTSGGTAA |
| Spy: | | LGCLVKDYFPEPVTVSWNSGALTSGVHTFPAVLQSSGLYSLSSVVTVPSSSLGTQT |
| | | YICNVNHKPSNTKVDKKVEPKSCDKTHTCPPCPAPNAAGGPSVFIFPPKIKDVLMI |
| | | SLSPIVTCVVVDVSEDDPDVQISWFVNNVEVHTAQTQTHREDYNSTLRVVSALPI |
| | | QHQDWMSGKEFKCKVNNKDLGAPIERTISKPKGSVRAPQVYVLPPPEEEMTKKQ |
| | | VTLTCMVTDFMPEDIYVEWTNNGKTELNYKNTEPVLDSDGSYFMYSKLRVEKKN |
| | | WVERNSYSCSVVHEGLHNHHTTKSFSRTPGGSGGSGGSGRGVPHIVMVDAYKRY |
| | | K |
| 48D_H. | 51 | MGWSCIILFLVATATGVHSEVQLVQSGAEVKKPGATVKISCKASGYTFSDFYMY |
| ShortUpperHinge. | | WVRQAPGKGLEWMGLIDPEDACTMYAEKFRGRVTITADTSTDTGYLELSSLRSE |
| MouseIgG2a. | | DTAVYYCAADPWELNAFNVWGQGTLVSVSSASTKGPSVFPLAPSSKSTSGGTAA |
| Kde1.Spy | | LGCLVKDYFPEPVTVSWNSGALTSGVIITFPAVLQSSGLYSLSSVVTVPSSSLGTQT |
| | | YICNVNHKPSNTKVDKKVEPKSCDKTHTCPPCPAPNAAGGPSVFIFPPKIKDVLMI |
| | | SLSPIVTCVVVDVSEDDPDVQISWFVNNVEVHTAQTQTHREDYNSTLRVVSALPI |
| | | QHQDWMSGKEFKCKVNNKDLGAPIERTISKPKGSVRAPQVYVLPPPEEEMTKKQ |
| | | VTLTCMVTDFMPEDIYVEWTNNGKTELNYKNTEPVLDSDGSYFMYSKLRVEKKN |
| | | WVERNSYSCSVVHEGLHNHHTTKSFSRTPGGSGGSGGSGRGVPHIVMVDAYKRY |
| | | K |
| gp120.Core. | 61 | MPMGSLQPLATLYLLGMLVASVLASAITQACPKVSFEPIPIHYCAPAGFAILKCKD |
| 48D + 48D. | | KKFNGTGPCPSVSTVQCTHGIKPVVSTQLLLNGSLAEEEVMIRSENITNNAKNILV |
| Light_Fusion | | QFNTPVQINCTRPGNGGDIRQAHCNVSKATWNETLGKVVKQLRKHFGNNTIIRFA |
| | | NSSGGDLEVTTHSFNCGGEFFYCNTSGLFNSTWISNTSVQGSNSTGSNDSITLPCRI |
| | | KQCINMWQRIGQAMYAPPIQGVIRCVSNITGLILTRDGGSTNSTTETFRPGGGDMR |
| | | DNWRSELYKYKVVKIEGGVWKDAETTLFCASDAKAYETEKHNVWATHACVPTD |
| | | PNPQEIHLEGVTEEFNMWKNNMVEQMHTDIISLWDQSLKPCVKLTGGGSGGGSG |
| | | GGSDIQMTQSPSSVSASVGDRVTITCRASQDISTWLAWYQQKPGKAPKLLIYAAS |
| | | TLQSGVPSRFSGSGSGTDFSLTINSLQPEDFATYYCQQANSFFTFGGGTKVEIKRTV |
| | | AAPSVFIFPPSDEQLKSGTASVVCLLNNFYPREAKVQWKVDNALQSGNSQESVTE |
| | | QDSKDSTYSLSSTLTLSKADYEKHKVYACEVTHQGLSSPVTKSFNRGEC |
| RFD2_v2_ | 63 | MGWSCIILFLVATATGVHSEVQLVQSGAEVKKPGATVKISCKASGYTFSDFYMY |
| Heavy | | WVRQAPGKGLEWMGLIDPEDACTMYAEKFRGRVTITADTSTDTGYLELSSLRSE |
| | | DTAVYYCAADPWELNAFNVWGQGTLVSVSGAAVTGPRVFPLSPAGCSTTSPTTL |
| | | LGCLVDDYYPLPVTVTWNGGALTSGVTTFPAVLRPDGLYSQVSVISVPSSSCGTE |
| | | TYTCNVTHPASNTTVSQLVPCPRQLELPAEALAHLRAFLALLPPAAALAVAHAVL |
| | | QALAAAL |
| RFD2_v2_Light | 65 | MGWSCIILFLVATATGVHSDIQMTQSPSSVSASVGDRVTITCRASQDISTWLAWY |
| | | QQKPGKAPKILIYAASTLQSGVPSRFSGSGSGTDFSLTINSLQPEDFATYYCQQAN |
| | | SFFTFGGGTKVEIKRTVVAPSVSIFPPSPEELASGRARVVCVASSFYPESLEVTWLV |
| | | DGEPVSGRSFTSTSAKNPADNTYSLVSTLDLSTEEYLAHRRFECRVTHSGLSSPVT |
| | | ASILRVGDTLFTSVSSSDDGVFTVTGSPLMQLALDLYLEAQAA |
| RFD3_v2_ | 67 | MGWSCIILFLVATATGVHSEVQLVQSGAEVKKPGATVKISCKASGYTFSDFYMY |
| Heavy | | WVRQAPGKGLEWMGLIDPEDACTMYAEKFRGRVTITADTSTDTGYLELSSLRSE |
| | | DTAVYYCAADPWELNAFNVWGQGTLVSVSGAPVTGPEVFPLPPMSVGSGKPSAT |
| | | LGCLVKNFYPLPVEVTWNGGAVTEGVYVFPPVLRPDGRWSLVSVLRVPESTLGT |
| | | ETYVCNVRHPPANVTVSKEVKALPGVTLTQTPEDAALEAKLRKACPEAEAIIVGT |
| | | LPNGATLVLVFVP |
| RFD3_v2_Light | 69 | MGWSCIILFLVATATGVHSDIQMTQSPSSVSASVGDRVTITCRASQDISTWLAWY |
| | | QQKPGKAPKLLIYAASTLQSGVPSRFSGSGSGTDFSLTINSLQPEDFATYYCQQAN |
| | | SFFTFGGGTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVTHQGLSSP |
| | | VTKSFNKYPGVVVTETVDEATGTTTITLNTKNYKAVRLAIKIAEALHKLRPGSTTV |
| | | AVIS |
| gp120-BAR | 70 | LEAKAKALKAALDAAAAAAAAAAAAYLALLAATEAAVAAALAKIPDPATRAAR |
| | | AALAAAFAAALRGFATDLIAGGYAGLKERAEEIVELEKERAALAARRADLAAQA |
| | | AALAALEAEGTRLQRELRGYLAAIKGMQEASMKLTESLHEVYEPDWYGREDVK |
| | | MVGEKCDVLWEDFHQKLVDESLLTLDTYLGQFPALRARLAALAAQAAALEAAL |
| | | QALIDLLQKKPYGDAETTLFCASDAKAYETEKHNVWATHACVPAVPRFETPALP |
| | | VTEEFNMWKNNMVEQMHTDIISLWDQSLKPCVKLTPLCNCNTSAITQACPKVSFE |
| | | PIPIHYCAPAGFAILKCKDKKFNGTGPCPSVSTVQCTHGIKPVVSTQLLLNGSLAEE |
| | | EVMIRSENITNNAKNILVQFNTPVQINCTRPNDIIGDIRQAHCNVSKATWNETLGK |
| | | VVKQLRKHFGNNTIIRFANSSGGDLEVTTHSFNCGGEFFYCNTSGLFNSTWISNND |
| | | SITLPCRIKQIINMWQRIGQAMYAPPIQGVIRCVSNITGLILTRDGGSTNSTTETFRP |
| | | GGGDMRDNWRSELYKYKVVKPLSEDPELKAARAAYAAARAAYDAAYDALAAE |
| | | LPALLSRRVGFYVNTFKNVSSLEAKFHKEIAVLCHKLYEVMTKLGDQHA |
| 1gh7_Fusion_ | 71 | EETIPLQTLRCYNDYTSHITCRWADTQDAQRLVNVTLIRRVNEDLLEPVSCDLSDD |
| 008 | | MPWSACPHPRCVPRRCVIPCQSFVVTDVDYFSFQPDRPLGTRLTVTLTQHVQPPEP |
| | | RDLQISTDQDHFLLTWSVALGSPQSHWLSPGDLEFEVVYKRLQDSWEDAAILLSN |
| | | TSQATLGPEHLMPSSTYVARVRTRLAPGSRLSGRPSKWSPEVCWDSQPGDEAQPQ |
| | | NLECFFDGAAVLSCSWEVRKEVASSVSFGLFYKPSPREEECSPVLREGLGSLHTRH |
| | | HCQIPVPDPATHGQYIVSVQPRRAEKHIKSSVNPTYYETFPNYQVIYLPTTTTDPSN |
| | | KEPLGNTVTPAPVGTVTTYTPDGTRSSAITQACPKVTFEPIPIHYCAPAGFAILKCK |
| | | DEEFNGTGLCKNVSTVQCTHGIKPVVSTQLLLNGSLAEKEIRIKSENISDNAKTIIV |
| | | QLTKPVLINCARPSGDIRQAYCVVNRTQWNDTLGQVAIQLRKHWNTTIIFNEPSG |
| | | GDLEITTHSFNCGGEFFYCNTSDLFNSTWNDNITLPCRIKQIINMWQRVGQAMYAP |
| | | PIQGVIRCQSNITGILLTRDGGNTRTNETFRPGGGDMRDNWRSELYKYKVVKIE |

### Divalent Immunogen Antigens

The divalent scaffolds disclosed herein may be especially well suited for presentation of antigens from diseases or conditions for which eliciting a protective or therapeutic immune response has proven challenging. Non-limiting examples of such diseases include viruses for which elicitation of an immune response comprising a preferred neutralizing antibody (e.g., a broadly neutralizing antibody) would be expected to be protective or therapeutic. Additional non-limiting examples of such diseases include rapidly-mutating viruses. Non-limiting examples of such viruses include human immunodeficiency virus (HIV), influenza A, influenza B, severe acute respiratory syndrome coronaviruses (SARS-COV-1 and SARS-CoV-2), sarbecoviruses, hepatitis C virus, respiratory syncytial virus, dengue virus, Ebola virus, and norovirus.

Suitable antigens for presentation as part of the divalent immunogens described herein may include those exposed on the surface of a virus. Suitable antigens may also include any antigens recognized by neutralizing antibodies which, if elicited, would be expected to contribute to a protective or therapeutic immune response. In some embodiments, the antigen may be a viral spike protein, envelope protein, membrane protein, or fusion protein. Non-limiting examples of antigens for presentation by the disclosed divalent immunogens include HIV envelope protein, Influenza A Hemagglutinin, Influenza B Hemagglutinin, SARS-COV-2 spike protein, hepatitis C virus E2 protein, respiratory syncytial virus fusion (F) protein, and Ebola virus glycoprotein. In some embodiments, the antigen comprises a sequence at least 50%, at least 55%, at least 60%, at least 65%, at least 70%, at least 75%, at least 80%, at least 85%, at least 90%, at least 92%, at least 95%, at least 98%, or at least 99% identical to any one of SEQ ID NOs: 13, 15, and 54-60. In some embodiments, the antigen comprises any one of SEQ ID NOs: 13, 15, and SEQ ID NOs: 54-60.

In some embodiments, the antigen comprises HIV gp120, or HIV gp120 Core, or a fragment or variant thereof. In some embodiments, the antigen comprises a sequence at least 50%, at least 55%, at least 60%, at least 65%, at least 70%, at least 75%, at least 80%, at least 85%, at least 90%, at least 92%, at least 95%, at least 98%, or at least 99% identical to SEQ ID NO: 13. In some embodiments, the antigen comprises SEQ ID NO: 13. In some embodiments, the antigen comprises SEQ ID NO: 15.

In some embodiments, the epitope is the CD4 binding site of HIV gp120, or a fragment or variant thereof. In some embodiments, the epitope comprises a sequence at least 50%, at least 55%, at least 60%, at least 65%, at least 70%, at least 75%, at least 80%, at least 85%, at least 90%, at least 92%, at least 95%, at least 98%, or at least 99% identical to residues 123-124, 275-282, 365-371, 427-432, and 455-476 of SEQ ID NO: 53. In some embodiments, the epitope comprises residues corresponding to amino acids 123-124, 275-282, 365-371, 427-432, and 455-476 of SEQ ID NO: 53.

In some embodiments, presentation of non-identical but genetically related antigens may be advantageous. In a non-limiting example, presentation of non-identical but genetically related antigens may be advantageous for the divalent, high-avidity binding of BCRs that have broad reactivity. In this setting, the inclusion of two antigens from distantly related isolates of the same virus, each bound to the scaffold, would result in high avidity, divalent binding by BCRs that are able to bind to conserved features between the strains, which can promote broad reactivity.

In some embodiments, the first antigen or epitope thereof is 100% identical to the second antigen or epitope thereof. In some embodiments, the first antigen or epitope thereof is less than 100% identical to the second antigen or epitope thereof. For example, the first antigen or epitope thereof may be at least 50%, at least 60%, at least 70%, at least 80%, at least 90%, at least 95%, at least 98%, or at least 99% identical to the second antigen or epitope thereof.

Percentage “sequence identity” of one polypeptide with respect to another polypeptide refers to the percentage of amino acids of the first polypeptide that are the same as the second polypeptide, when comparing the two aligned sequences. Sequence similarity can be determined via a number of different methods. For example, sequence similarity or identity may be determined using the local sequence identity algorithm of Smith & Waterman, Adv. Appl. Math. 2, 482 (1981), by the sequence identity alignment algorithm of Needleman & Wunsch J Mol. Biol. 48, 443 (1970), by the search for similarity method of Pearson & Lipman, Proc. Natl. Acad. Sci. USA 85, 2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al. Nucl. Acid Res. 12, 387-395 (1984), or by inspection.

Another suitable algorithm is the BLAST algorithm, described in Altschul et al. J. Mol. Biol. 215, 403-410, (1990) and Karlin et al. Proc. Natl. Acad. Sci. USA 90, 5873-5787 (1993). A particularly useful BLAST program is the WU-BLAST-2 program which was obtained from Altschul et al. Methods in Enzymology, 266, 460-480 (1996); blast.wust1/edu/blast/README.html. WU-BLAST-2 uses several search parameters, which are optionally set to the default values. The parameters are dynamic values and are established by the program itself depending upon the composition of the particular sequence and composition of the particular database against which the sequence of interest is being searched; however, the values may be adjusted to increase sensitivity. Further, an additional useful algorithm is gapped BLAST as reported by Altschul et al, (1997) Nucleic Acids Res. 25, 3389-3402. Unless otherwise indicated, percent identity is determined herein using the algorithm available at the internet address: blast.ncbi.nlm.nih.gov/Blast.cgi.

In some embodiments, each antigen or epitope thereof comprises a first engineered antigen mutation or a first engineered epitope mutation. In some embodiments, the first engineered antigen mutation or first engineered epitope mutation enables formation of a disulfide bond between the scaffold and the antigen or epitope thereof. Non-limiting examples of engineered mutations include addition, deletion, substitution, or insertion of one or more amino acids into the amino acid sequence of the Fab fragment. In some embodiments, the first engineered antigen mutation corresponds to 1423C of SEQ ID NO: 53.

Incorporation of heterodimeric antigens into the divalent immunogen may be achieved via a number of mechanisms. A non-limiting example of such a mechanism is through use of bivalent antibody technology, where asymmetry is engineered into the complex (e.g., in the Fc region or any other scaffold region) such that only heterodimers (e.g., of an antigen-Fab fusion protein, for example) can stably form. Other non-limiting examples of such a mechanism are to attach a different affinity tag to each half of the antibody and then purify for both tags, or to engineer the heterodimeric antigen on a single polypeptide chain.

In some embodiments, the first and second antigen of the divalent immunogen differ at least in the presence of specific glycans. For example, a glycan at a specific position (e.g., a specific amino acid) can be present on the first antigen and absent on the second antigen, or present on the second antigen and absent on the first antigen. A glycan can also be present on both the first and second antigens or absent on both the first and second antigens. A non-limiting example of such a glycan is the glycan at position 276 of HIV antigen gp120. In some embodiments, the first gp 120 antigen comprises a glycan at position 276 and the second gp120 antigen does not comprise a glycan at position 276. In some embodiments, the first gp120 antigen does not comprise a glycan at position 276 and the second gp120 antigen comprises a glycan at position 276. It is well-established that a major obstacle for VRC01 lineage B-cells is overcoming the steric restriction caused by a glycan at this position. Immunogens that uniformly incorporate this glycan across all constituting gp120 antigens may offer an insurmountable challenge to the available BCR repertoire of VRC01-lineage B-cells. Divalent antigens that that differ in glycan occupancy at position 276 may provide an intermediate level of avidity such that B-cells whose receptors gain mutations marginally effective at accommodating the glycan at that position are positively selected.

Non-limiting examples of sequences that the divalent immunogen antigens may comprise are shown in Table 2 and SEQ ID NOs: 13-16 and SEQ ID NOs: 53-59.

TABLE 2
Non-limiting examples of polypeptides comprised by divalent immunogen
antigens

| gp120. | SEQ ID | |
|---|---|---|
| Core | NO. | Sequence |
| gp120. | 13 | MPMGSLQPLATLYLLGMLVASVLAVWKDAETTLFCASDAKAYETEKHNVWATHAC |
| Core | | VPTDPNPQEIHLEGVTEEFNMWKNNMVEQMHTDIISLWDQSLKPCVKLTGGSAITQA |
| | | CPKVSFEPIPIHYCAPAGFAILKCKDKKFNGTGPCPSVSTVQCTHGIKPVVSTQLLLNG |
| | | SLAEEEVMIRSENITNNAKNILVQFNTPVQINCTRPGNGGDIRQAHCNVSKATWNETL |
| | | GKVVKQLRKHFGNNTIIRFANSSGGDLEVTTHSFNCGGEFFYCNTSGLFNSTWISNTS |
| | | VQGSNSTGSNDSITLPCRIKQIINMWQRIGQAMYAPPIQGVIRCVSNITGLILTRDGCST |
| | | NSTTETFRPGGGDMRDNWRSELYKYKVVKIE |
| gp120. | 14 | ATGCCCATGGGCTCCCTGCAGCCGTTAGCAACTCTGTATCTGCTGGGGATGCTGGT |
| Core. | | AGCCAGCGTGCTGGCAGTGTGGAAAGATGCTGAAACCACATTGTTTTGTGCGAGC |
| 48D | | GATGCCAAAGCATACGAGACGGAAAAGCACAATGTCTGGGCCACTCATGCATGC |
| | | GTGCCCACAGACCCAAACCCCCAGGAGATCCACCTGGAAGGAGTGACCGAGGAA |
| | | TTCAACATGTGGAAAAACAATATGGTGGAGCAGATGCATACAGACATCATTAGCC |
| | | TGTGGGATCAGTCCCTGAAGCCCTGCGTCAAACTGACTGGCGGCTCCGCCATTAC |
| | | CCAGGCTTGTCCTAAAGTGTCTTTTGAGCCTATCCCAATTCATTATTGCGCCCCAG |
| | | CTGGCTTCGCCATCCTGAAGTGTAAAGATAAGAAGTTCAACGGAACTGGCCCCTG |
| | | CCCTTCCGTGTCTACAGTCCAGTGTACTCACGGGATTAAGCCTGTGGTCTCTACAC |
| | | AGCTGCTGCTGAATGGAAGTCTGGCTGAGGAAGAAGTGATGATCCGGAGCGAGA |
| | | ACATTACCAACAATGCCAAGAATATCCTGGTCCAGTTCAACACACCAGTGCAGAT |
| | | TAATTGCACAAGACCCGGAAATGGAGGAGATATCAGACAGGCTCACTGTAATGTG |
| | | AGTAAGGCAACCTGGAACGAGACACTGGGCAAGGTGGTCAAACAGCTGAGGAAA |
| | | CATTTCGGGAATAACACCATCATTCGCTTTGCCAATAGCTCCGGAGGGGACCTGG |
| | | AGGTCACTACCCACTCCTTCAACTGCGGAGGCGAATTCTTTTACTGTAACACATCT |
| | | GGCCTGTTTAATAGTACATGGATCTCTAACACTAGTGTGCAGGGCAGTAATTCAA |
| | | CTGGGTCAAACGATAGCATCACCCTGCCATGCCGAATTAAGCAGATCATTAATAT |
| | | GTGGCAGCGGATAGGTCAGGCAATGTATGCTCCCCCAATCCAGGGAGTTATTCGG |
| | | TGCGTCTCTAATATCACGGGACTTATCTTGACTCGCGACGGCTGCAGCACGAACA |
| | | GCACAACAGAAACATTCCGGCCTGGGGGGGGAGACATGCGGGATAACTGGCGGA |
| | | GTGAGCTGTACAAGTATAAAGTTGTTAAGATCGAATGA |
| gp120. | 15 | MPMGSLQPLATLYLLGMLVASVLAVWKDAETTLFCASDAKAYETEKHNVWATHAC |
| Core. | | VPTDPNPQEIHLEGVTEEFNMWKNNMVEQMHTDIISLWDQSLKPCVKLTGGSAITQA |
| 48D | | CPKVSFEPIPIHYCAPAGFAILKCKDKKFNGTGPCPSVSTVQCTHGIKPVVSTQLLLNG |
| | | SLAEEEVMIRSENITNNAKNILVQFNTPVQINCTRPGNGGDIRQAHCNVSKATWNETL |
| | | GKVVKQLRKHFGNNTIIRFANSSGGDLEVTTHSFNCGGEFFYCNTSGLFNSTWISNTS |
| | | VQGSNSTGSNDSITLPCRIKQCINMWQRIGQAMYAPPIQGVIRCVSNITGLILTRDGGS |
| | | TNSTTETFRPGGGDMRDNWRSELYKYKVVKIE |
| gp120 | 16 | ATGCCCATGGGCTCCCTGCAGCCGTTAGCAACTCTGTATCTGCTGGGGATGCTGGT |
| from | | AGCCAGCGTGCTGGCAGTGTGGAAAGATGCTGAAACCACATTGTTTTGTGCGAGC |
| HXBc2 | | GATGCCAAAGCATACGAGACGGAAAAGCACAATGTCTGGGCCACTCATGCATGC |
| SEQ | | GTGCCCACAGACCCAAACCCCCAGGAGATCCACCTGGAAGGAGTGACCGAGGAA |
| | | TTCAACATGTGGAAAAACAATATGGTGGAGCAGATGCATACAGACATCATTAGCC |
| | | TGTGGGATCAGTCCCTGAAGCCCTGCGTCAAACTGACTGGCGGCTCCGCCATTAC |
| | | CCAGGCTTGTCCTAAAGTGTCTTTTGAGCCTATCCCAATTCATTATTGCGCCCCAG |
| | | CTGGCTTCGCCATCCTGAAGTGTAAAGATAAGAAGTTCAACGGAACTGGCCCCTG |
| | | CCCTTCCGTGTCTACAGTCCAGTGTACTCACGGGATTAAGCCTGTGGTCTCTACAC |
| | | AGCTGCTGCTGAATGGAAGTCTGGCTGAGGAAGAAGTGATGATCCGGAGCGAGA |
| | | ACATTACCAACAATGCCAAGAATATCCTGGTCCAGTTCAACACACCAGTGCAGAT |
| | | TAATTGCACAAGACCCGGAAATGGAGGAGATATCAGACAGGCTCACTGTAATGTG |
| | | AGTAAGGCAACCTGGAACGAGACACTGGGCAAGGTGGTCAAACAGCTGAGGAAA |
| | | CATTTCGGGAATAACACCATCATTCGCTTTGCCAATAGCTCCGGAGGGGACCTGG |
| | | AGGTCACTACCCACTCCTTCAACTGCGGAGGCGAATTCTTTTACTGTAACACATCT |
| | | GGCCTGTTTAATAGTACATGGATCTCTAACACTAGTGTGCAGGGCAGTAATTCAA |
| | | CTGGGTCAAACGATAGCATCACCCTGCCATGCCGAATTAAGCAGTGCATTAATAT |
| | | GTGGCAGCGGATAGGTCAGGCAATGTATGCTCCCCCAATCCAGGGAGTTATTCGG |
| | | TGCGTCTCTAATATCACGGGACTTATCTTGACTCGCGACGGCGGCAGCACGAACA |
| | | GCACAACAGAAACATTCCGGCCTGGGGGGGGAGACATGCGGGATAACTGGCGGA |
| | | GTGAGCTGTACAAGTATAAAGTTGTTAAGATCGAATGA |
| HIV | 53 | MRVKEKYQHLWRWGWRWGTMLLGMLMICSATEKLWVTVYYGVPVWKEATTTLF |
| Envelope | | CASDAKAYDTEVHNVWATHACVPTDPNPQEVVLVNVTENFNMWKNDMVEQMHED |
| protein | | IISLWDQSLKPCVKLTPLCVSLKCTDLKNDTNTNSSSGRMIMEKGEIKNCSFNISTSIRG |
| | | KVQKEYAFFYKLDIIPIDNDTTSYKLTSCNTSVITQACPKVSFEPIPIHYCAPAGFAILKC |
| | | NNKTFNGTGPCTNVSTVQCTHGIRPVVSTQLLLNGSLAEEEVVIRSVNFTDNAKTIIVQ |
| | | LNTSVEINCTRPNNNTRKRIRIQRGPGRAFVTIGKIGNMRQAHCNISRAKWNNTLKQI |
| | | ASKLREQFGNNKTIIFKQSSGGDPEIVTHSFNCGGEFFYCNSTQLFNSTWFNSTWSTEG |
| | | SNNTEGSDTITLPCRIKQIINMWQKVGKAMYAPPISGQIRCSSNITGLLLTRDGGNSNN |
| | | ESEIFRPGGGDMRDNWRSELYKYKVVKIEPLGVAPTKAKRRVVQREKRAVGIGALFL |
| | | GFLGAAGSTMGAASMTLTVQARQLLSGIVQQQNNLLRAIEAQQHLLQLTVWGIKQL |
| | | QARILAVERYLKDQQLLGIWGCSGKLICTTAVPWNASWSNKSLEQIWNHTTWMEWD |
| | | REINNYTSLIHSLIEESQNQQEKNEQELLELDKWASLWNWFNITNWLWYIKLFIMIVG |
| | | GLVGLRIVFAVLSIVNRVRQGYSPLSFQTHLPTPRGPDRPEGIEEEGGERDRDRSIRLV |
| | | NGSLALIWDDLRSLCLFSYHRLRDLLLIVTRIVELLGRRGWEALKYWWNLLQYWSQE |
| | | LKNSAVSLLNATAIAVAEGTDRVIEVVQGACRAIRHIPRRIRQGLERILL |
| Influenza | 54 | MKAKLFVLFCAFNALKADTICVGYHANNSTDTVDTILKKNVTVTHSVNLLENTHNG |
| A | | KLCSLNGKAPLQLENCNVAGWILGNPECDFLLTENSWSYIIETSNSRNGTCYPGEFTN |
| Hemagglutinin | | YEELREQLSTVSSFERFEIFPKVTSWPSHETTKGTTVACSHSGANSFYRNLLWIVKKGD |
| | | SYPKLSKSYTNNKGKEVLVIWGVHHPPTYSDQQTLYQNNHTYVSVGSSKYYQRFTPE |
| | | IVARPKIREQAGRMNYYWLLLDQGDTITFEATGNLIAPWHAFALDKGSNSGIMMSDA |
| | | HVRNCTTKCQTPHGALKSNLPFQNVHPITIGECPKYVKSTHLRMATGLRNIPSIQSRGL |
| | | FGAIAGFIEGGWTGMIDGWYGYHHQNEQGSGYAADKKSTQIAIDGISNKVNSIIEKM |
| | | NIQFTSVGKEFNNLEKRIENLNKKVDDGFLDVWTYNAELLILLENERTLDFHDFNVRN |
| | | LYERVKSQLRNNAKEIGNGCFEFYHKCDNECMESVKNGTYNYPRYSEESKLNREKID |
| | | GVKLESLGVHQILAIYSTVASSLILLVSLGAIVFWMCSNGSLQCRICI |
| Influenza | 55 | MKAKLFVLFCAFNALKADTICVGYHANNSTDTVDTILKKNVTVTHSVNLLENTHNG |
| B | | KLCSLNGKAPLQLENCNVAGWILGNPECDFLLTENSWSYIIETSNSRNGTCYPGEFTN |
| IIemagglutinin | | YEELREQLSTVSSFERFEIFPKVTSWPSHIETTKGTTVACSIISGANSFYRNLLWIVKKGD |
| | | SYPKLSKSYTNNKGKEVLVIWGVHHPPTYSDQQTLYQNNHTYVSVGSSKYYQRFTPE |
| | | IVARPKIREQAGRMNYYWLLLDQGDTITFEATGNLIAPWHAFALDKGSNSGIMMSDA |
| | | HVRNCTTKCQTPHGALKSNLPFQNVHPITIGECPKYVKSTHLRMATGLRNIPSIQSRGL |
| | | FGAIAGFIEGGWTGMIDGWYGYHHQNEQGSGYAADKKSTQIAIDGISNKVNSIIEKM |
| | | NIQFTSVGKEFNNLEKRIENLNKKVDDGFLDVWTYNAELLILLENERTLDFHDFNVRN |
| | | LYERVKSQLRNNAKEIGNGCFEFYHKCDNECMESVKNGTYNYPRYSEESKLNREKID |
| | | GVKLESLGVHQILAIYSTVASSLILLVSLGAIVFWMCSNGSLQCRICI |
| SARS- | 56 | MKAIIVLLMVVTSNADRICTGITSSNSPHVVKTATQGEVNVTGVIPLTTTPTKSYFANL |
| CoV-2 | | KGTRTRGKLCPDCLNCTDLDVALGRPMCVGTTPSAKASILHEVKPVTSGCFPIMHDR |
| Spike | | TKIRQLPNLLRGYENIRLSTQNVIDAEKAPGGPYRLGTSGSCPNATSKSGFFATMAWA |
| | | VPKDNNKNATNPLTVEVPYICTEGEDQITVWGFHSDDKTQMKNLYGDSNPQKFTSSA |
| | | NGVTTHYVSQIGSFPDQTEDGGLPQSGRIVVDYMMQKPGKTGTIVYQRGVLLPQKV |
| | | WCASGRSKVIKGSLPLIGEADCLHEKYGGLNKSKPYYTGEHAKAIGNCPIWVKTPLK |
| | | LANGTKYRPPAKLLKERGFFGAIAGFLEGGWEGMIAGWHGYTSHGAHGVAVAADL |
| | | KSTQEAINKITKNLNSLSELEVKNLQRLSGAMDELHNEILELDEKVDDLRADTISSQIE |
| | | LAVLISNEGIINSEDEHLLALERKLKKMLGPSAVEIGNGCFETKHKCNQTCLDRIAAG |
| | | TFNAGEFSLPTFDSLNITAASLNDDGLDNHTILLYYSTAASSLAVTLMLAIFIVYMVSR |
| | | DNVSCSICL |
| Hepatitis C | 57 | MFVFLVLLPLVSSQCVNLTTRTQLPPAYTNSFTRGVYYPDKVFRSSVLHSTQDLFLPFF |
| Virus | | SNVTWFHAIHVSGTNGTKRFDNPVLPFNDGVYFASTEKSNIIRGWIFGTTLDSKTQSLL |
| E2 | | IVNNATNVVIKVCEFQFCNDPFLGVYYHKNNKSWMESEFRVYSSANNCTFEYVSQPF |
| | | LMDLEGKQGNFKNLREFVFKNIDGYFKIYSKHTPINLVRDLPQGFSALEPLVDLPIGINI |
| | | TRFQTLLALHRSYLTPGDSSSGWTAGAAAYYVGYLQPRTFLLKYNENGTITDAVDCA |
| | | LDPLSETKCTLKSFTVEKGIYQTSNFRVQPTESIVRFPNITNLCPFGEVFNATRFASVYA |
| | | WNRKRISNCVADYSVLYNSASFSTFKCYGVSPTKLNDLCFTNVYADSFVIRGDEVRQI |
| | | APGQTGKIADYNYKLPDDFTGCVIAWNSNNLDSKVGGNYNYLYRLFRKSNLKPFER |
| | | DISTEIYQAGSTPCNGVEGENCYFPLQSYGFQPINGVGYQPYRVVVLSFELLHAPATV |
| | | CGPKKSTNLVKNKCVNFNFNGLTGTGVLTESNKKFLPFQQFGRDIADTTDAVRDPQT |
| | | LEILDITPCSFGGVSVITPGTNTSNQVAVLYQDVNCTEVPVAIHADQLTPTWRVYSTGS |
| | | NVFQTRAGCLIGAEHVNNSYECDIPIGAGICASYQTQTNSPRRARSVASQSIIAYTMSL |
| | | GAENSVAYSNNSIAIPTNFTISVTTEILPVSMTKTSVDCTMYICGDSTECSNLLLQYGSF |
| | | CTQLNRALTGIAVEQDKNTQEVFAQVKQIYKTPPIKDFGGFNFSQILPDPSKPSKRSFIE |
| | | DLLFNKVTLADAGFIKQYGDCLGDIAARDLICAQKFNGLTVLPPLLTDEMIAQYTSAL |
| | | LAGTITSGWTFGAGAALQIPFAMQMAYRENGIGVTQNVLYENQKLIANQFNSAIGKIQ |
| | | DSLSSTASALGKLQDVVNQNAQALNTLVKQLSSNFGAISSVLNDILSRLDKVEAEVQI |
| | | DRLITGRLQSLQTYVTQQLIRAAEIRASANLAATKMSECVLGQSKRVDFCGKGYHLM |
| | | SFPQSAPHGVVFLHVTYVPAQEKNFTTAPAICHDGKAHFPREGVFVSNGTHWFVTQR |
| | | NFYEPQIITTDNTFVSGNCDVVIGIVNNTVYDPLQPELDSFKEELDKYFKNHTSPDVDL |
| | | GDISGINASVVNIQKEIDRLNEVAKNLNESLIDLQELGKYEQYIKWPWYIWLGFIAGLI |
| | | AIVMVTIMLCCMTSCCSCLKGCCSCGSCCKFDEDDSEPVLKGVKLHYT |
| Respiratory | 58 | VDAETHVTGGNAGRTTAGLVGLLTPGAKQNIQLINTNGSWHINSTALNCNESLNTGW |
| Syncytial | | LAGLFYQHKFNSSGCPERLASCRRLTDFAQGWGPISYANGSGLDERPYCWHYPPRPC |
| Virus F | | GIVPAKSVCGPVYCFTPSPVVVGTTDRSGAPTYSWGANDTDVFVLNNTRPPLGNWFG |
| | | CTWMNSTGFTKVCGAPPCVIGGVGNNTLLCPTDCFRKYPEATYSRCGSGPRITPRCM |
| | | VDYPYRLWHYPCTINYTIFKVRMYVGGVEHRLEAACNWTRGERCDLEDRDRSELSP |
| | | LLLSTTQWQVLPCSFTTLPALSTGLIHLHQNIVDVQYLYGVGSSIASWAIKWEYVVLL |
| | | FLLLADARVCSCLW |
| Ebola | 59 | MELLILKANAITTILTAVTFCFASGQNITEEFYQSTCSAVSKGYLSALRTGWYTSVITIE |
| virus | | ISNIKENKCNGTDAKVKLIKQELDKYKNAVTELQLLMQSTPATNNRARREIPRFMNY |
| glycoprotein | | TLNNAKKTNVTLSKKQKQQAIASGVAVSKVLHLEGEVNKIKSALLSTNKAVVSLSNG |
| | | VSVLTSKVLDLKNYIDKQLLPIVNKQSCSISNIETVIEFQQKNNRLLEITREFSVNAGVT |
| | | TPVSTYMLTNSELLSLINDMPITNDQKKLMSNNVQIVRQQSYSIMSIIKEEVLAYVVQL |
| | | PLYGVIDTPCWKLHTSPLCTTNTKEGSNICLTRTDRGWYCDNAGSVSFFPQAETCKVQ |
| | | SNRVFCDTMNSLTLPSEVNLCNVDIFNPKYDCKIMTSKTDVSSSVITSLGAIVSCYGKT |
| | | KCTASNKNRGIIKTFSNGCDYVSNKGVDTVSVGNTLYYVNKQEGKSLYVKGEPIINFY |
| | | DPLVFPSDEFDASISQVNEKINQSLAFIRKSDELLHNVNAGKSTTNIMITTIIIVIIVILLSL |
| | | IAVGLLLYCKARSTPVTLSKDQLSGINNIAFSN |

### Antigenic Distance in Divalent Immunogens

Fab-Fab geometry of an IgG antibody has a strong impact on the avidity conveyed by divalent binding, especially for antibodies with low monovalent affinity. For the divalent immunogens disclosed herein, antigenic spacing (the distance between the two antigens or epitopes thereof) is a key parameter considered for optimizing binding of a certain neutralizing antibody (e.g., a preferred neutralizing antibody and/or a broadly neutralizing antibody) to the antigens.

In some embodiments, the divalent immunogen provides a fixed and rigid spacing of the two antigens or epitopes thereof. In some embodiments, the divalent immunogen presents two antigens at a fixed antigenic distance. In some embodiments, the divalent immunogen provides an antigenic spacing of about 3 to about 20 nm. In some embodiments, the divalent immunogen provides an antigenic spacing of about 7 to about 19 nm. In some embodiments, the divalent immunogen provides an antigenic spacing of about 13 to about 16 nm. In some embodiments, the divalent immunogen provides an antigenic spacing of about 3, about 4, about 5, about 6, about 7, about 8, about 9, about 10, about 11, about 12, about 13, about 14, about 15, about 16, about 17, about 18, about 18, or about 20 nm. In some embodiments, the divalent immunogen provides an antigenic spacing of about 14 nm. In some embodiments, the divalent immunogen provides an antigenic spacing of about 15 nm. In some embodiments, the divalent immunogen provides an antigenic spacing of about 16 nm.

In some embodiments, the divalent immunogen provides precise positioning of two antigens or epitopes thereof such that certain (e.g, preferred) BCRs or neutralizing antibodies are preferably bound to both antigens simultaneously. In some embodiments, the divalent immunogen provides antigenic spacing that allows a certain (e.g, preferred) BCR or neutralizing antibody to bind divalently with 1:1 stoichiometry to bind both antigens of the divalent immunogen) in a relaxed state.

### Fab-Fab Angles of Preferred B-Cell Receptors Bound to the Divalent Immunogen

The present disclosure provides divalent immunogens that provide precise positioning of two antigens or epitopes thereof such that certain (e.g, preferred) BCRs and/or neutralizing antibodies bind to both antigens simultaneously under the most relaxed conformations of the BCR or antibody. In some embodiments, this relaxed conformation occurs with a certain angle (Fab-Fab angle) between the two Fab domains of the BCR or antibody. In some embodiments, two Fab domains of the bound BCR and/or antibody assume an angle of about 20 degrees to about 150 degrees. In some embodiments, the two Fab domains of the bound BCR and/or antibody assume an angle of about 90 degrees to about 140 degrees. In some embodiments, the two Fab domains of the bound BCR and/or antibody assume an angle of about 20, about 30, about 40, about 50, about 60, about 70, about 80, about 90, about 100, about 110, about 120, about 130, about 140, or about 150 degrees. In some embodiments, the two Fab domains of the bound BCR and/or antibody assume an angle of about 110, about 120, or about 130 degrees. In some embodiments, the two Fab domains of the bound BCR and/or antibody assume an angle of about 120 degrees.

The present disclosure also provides divalent immunogens that increase the binding avidity of certain (e.g., preferred) BCRs and/or antibodies. In some embodiments, the divalent immunogen increases the binding avidity of certain BCRs and/or antibodies due to the ability of the certain BCRs and/or antibodies to bind divalently (e.g., to bind to both antigens in the divalent immunogen). Divalent binding of the two antigens provides an avidity boost to certain BCRs and/or antibodies. This may be a particularly valuable mechanism for selectively binding certain BCRs and/or antibodies. For instance, this may be particularly valuable for BCRs and/or antibodies with low monovalent binding affinity.

In some embodiments, the binding avidity of certain BCRs and/or antibodies is at least 1.5, at least 5, at least 10, at least 20, at least 40, at least 60, at least 80, at least 100, at least 200, at least 400, at least 600, at least 800, or at least 1000-fold higher for the two antigens covalently linked to the scaffold compared to the same antigen unlinked to the scaffold. In some embodiments, the binding avidity of certain BCRs and/or antibodies is at least 1.5-fold higher for the two antigens covalently linked to the scaffold compared to the same antigen unlinked to the scaffold. In some embodiments, the binding avidity of certain BCRs and/or antibodies is at least 10-fold higher for the two antigens covalently linked to the scaffold compared to the same antigen unlinked to the scaffold. In some embodiments, the binding avidity of certain BCRs and/or antibodies is at least 100-fold higher for the two antigens covalently linked to the scaffold compared to the same antigen unlinked to the scaffold.

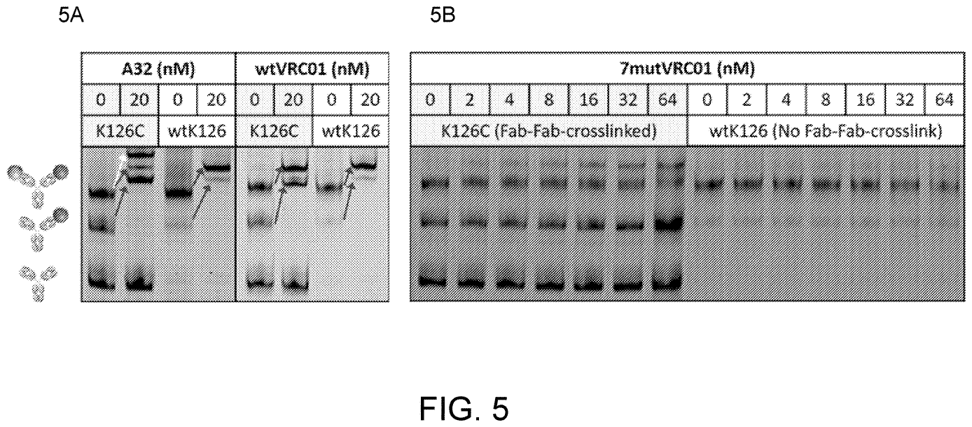

Monovalent and divalent binding of a BCR and/or antibody to the antigens of the divalent immunogen, avidity of a BCR and/or antibody for an antigen linked to a scaffold of a divalent immunogen, and/or avidity of a BCR and/or antibody not linked to a scaffold, may be measured by any method known in the art. For example, monovalent and divalent binding and/or avidity may be measured in a gel shift assay as shown in FIG. 5A and described in Example 2. Alternatively or additionally, avidity may be measured by surface plasmon resonance.

In some embodiments, the second engineered Fab mutation of the divalent immunogen fixes the distance between the antigens or epitopes thereof. In some embodiments, the second engineered Fab mutation modifies the interaction between the antigens or epitopes thereof and certain (e.g., preferred) BCRs and/or antibodies. In a non-limiting example, the second engineered Fab mutation may increase the avidity of certain (e.g., preferred) BCRs and/or antibodies for the antigens in the divalent immunogen. In a non-limiting example, the second engineered Fab mutation may decrease the avidity of other (e.g., non-preferred) BCRs and/or antibodies for the antigens in the divalent immunogen. In some embodiments, the second engineered Fab mutation enhances the binding avidity of certain BCRs and/or antibodies for the antigen or epitope thereof by at least 1.5-fold. In some embodiments, the second engineered Fab mutation enhances the binding avidity of certain BCRs and/or antibodies for the antigen or epitope thereof by at least 10-fold. In some embodiments, the second engineered Fab mutation enhances the binding avidity of certain BCRs and/or antibodies for the antigen or epitope thereof by at least 100-fold.

### Multimers of Divalent Immunogens

BCR crosslinking likely plays an important role in B-cell activation. The present disclosure provides multimers of the divalent immunogens describe herein. Multimerization at high or low valency may be effective for different divalent immunogen and/or different applications.

In some embodiments, the multimer is a dimer, a trimer, a tetramer, a 5-mer, a 6-mer, a 7-mer, an 8-mer, a 9-mer, or a 10-mer. In some embodiments, the multimer is a dimer, a trimer, or a tetramer.

The present disclosure also provides multimers of divalent immunogens conjugated onto a nanoparticle. A non-limiting example of a technology suitable for conjugating the divalent immunogens disclosed herein onto nanoparticles is the SpyCatcher/Spy Tag technology (Keeble et al., Proc Natl Acad Sci USA, 2019. 116(52): p. 26523-26533).

In some embodiments, multimers of divalent immunogens may be conjugated onto a nanoparticle by generating a SpyTagged divalent immunogen and contacting it with a SpyCatcher-VLP (Rahikainen et al. Angew Chem Int Ed Engl, 2021. 60(1): p. 321-330). In some embodiments, the SpyTagged divalent immunogen and SpyCatcher-VLP are mixed at an empirically determined molar ratio such that 1/3 of the SpyCatcher-VLP monomers are conjugated (VLP is made of 60 monomers of which 10 divalent constructs (20 SpyTags) can react).

In some embodiments, to generate dimers and trimers, two separate cultures are generated: one expressing the 15.6 kDa SpyCatcher at the divalent scaffold interface (heavy chain C-termini for Fab-based divalent immunogens) and one expressing a 50/50 combination of SpyTagged and untagged divalent complex resulting in a mixed species containing zero (25%), one (50%) or two (25%) SpyTags. Mixing of the two batches of divalent construct will result in dimers and trimers which can be separated by size exclusion chromatography (SEC) with Sephacryl-S300.

In some embodiments, dimers and/or trimers of the divalent immunogen maintain selective avidity and selective activation for preferred BCRs over low-affinity BCRs. In some embodiments, extensive multimerization (e.g., 10mer) of the divalent antigen results in loss of selective avidity/activation.

### Pharmaceutical Compositions

The present invention further contemplates a pharmaceutical composition comprising a divalent immunogen and one or more pharmaceutically acceptable diluent, carrier, or excipient.

The divalent immunogens can be combined with pharmaceutically-acceptable carriers, diluents, excipients and reagents useful in preparing a formulation that is generally safe, non-toxic, and desirable, and includes excipients that are acceptable for mammalian, e.g., human or primate, use. Such excipients can be solid, liquid, semisolid, or, in the case of an aerosol composition, gaseous. Examples of such carriers, diluents and excipients include, but are not limited to, water, saline, Ringer's solutions, dextrose solution, and 5% human serum albumin. Supplementary active compounds can also be incorporated into the formulations. Solutions or suspensions used for the formulations can include a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates; detergents such as Tween 20 to prevent aggregation; and compounds for the adjustment of tonicity such as sodium chloride or dextrose. The pH can be adjusted with acids or bases, such as hydrochloric acid or sodium hydroxide. In some embodiments, the pharmaceutical compositions are sterile.

Pharmaceutical compositions may further include sterile aqueous solutions or dispersions and sterile powders for the extemporaneous preparation of sterile injectable solutions or dispersion. For intravenous administration, suitable carriers include physiological saline, bacteriostatic water, or phosphate buffered saline (PBS). In some cases, the composition is sterile and should be fluid such that it can be drawn into a syringe or delivered to a subject from a syringe. In certain embodiments, it is stable under the conditions of manufacture and storage and is preserved against the contaminating action of microorganisms such as bacteria and fungi. The carrier can be, e.g., a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the internal compositions can be brought about by including in the composition an agent which delays absorption, for example, aluminum monostearate and gelatin.

Sterile solutions can be prepared by incorporating the divalent immunogen (or encoding polynucleotide) in the required amount in an appropriate solvent with one or a combination of ingredients enumerated above, as required, followed by filtered sterilization.

The pharmaceutical composition may additionally comprise an immunomodulatory component, such as an adjuvant. The adjuvant for administration in combination with a composition described herein may be administered before, concomitantly with, or after administration of said composition.

Non-limiting examples of adjuvants include aluminum salts such as aluminum hydroxide and/or aluminum phosphate; oil emulsion compositions (or oil-in-water compositions), including squalene-water emulsions, such as MF59 (see, for example, WO 90/14837); saponin formulations, for example, such as QS21 and immunostimulatory complexes (ISCOMS) (see, for example, U.S. Pat. No. 5,057,540; WO 90/03184, WO 96/11711, WO 2004/004762, WO 2005/002620); bacterial or microbial derivatives, examples of which are polyribosinic:polyribocytidic acid (polyI:C), monophosphoryl lipid A (MPL), 3-O-deacylated MPL (3 dMPL), CpG motif oligonucleotides, ADP-ribosylating bacterial toxins or their mutants, such as E. coli thermolabile enterotoxin LT, cholera toxin CT, etc.

### Polynucleotides

The present disclosure also provides polynucleotides encoding the scaffold and two antigens or epitopes thereof described herein. In some embodiments, a polynucleotide encodes a scaffold or portion thereof, wherein the encoded scaffold provides precise positioning of two antigens or epitopes thereof such that only certain (e.g., preferred) BCRs and/or antibodies are preferably bound to both antigens simultaneously. In some embodiments, a polynucleotide encodes an antigen or epitope thereof described therein. In some embodiments, a first polynucleotide comprises a sequence that encodes a heavy chain sequence and a second polynucleotide encodes a light chain sequence.

In some embodiments, the polynucleotide comprises a sequence that encodes a heavy chain sequence at least 80%, at least 85%, at least 90%, at least 92%, at least 95%, at least 98%, or at least 99% identical to the heavy chain sequence of any one of SEQ ID NOs: 1, 17, 19, 25, 29, 33, 37, 41, 45, 49, or 51. In some embodiments, the polynucleotide comprises a sequence that encodes a light chain sequence at least 80%, at least 85%, at least 90%, at least 92%, at least 95%, at least 98%, or at least 99% identical to the light chain sequence of any one of SEQ ID NOs: 3, 5, 7, 9, 11, 21, 23, 27, 31, 35, 39, 43, 47, and 62. In some embodiments, the polynucleotide comprises a sequence that encodes an antigen sequence at least 80%, at least 85%, at least 90%, at least 92%, at least 95%, at least 98%, or at least 99% identical to any one of SEQ ID NOs: 13, 15, 54, 55, 56, 57, 58, 59, and 60.

In some embodiments, the polynucleotide comprises a sequence that encodes a heavy chain sequence at least 80%, at least 85%, at least 90%, at least 92%, at least 95%, at least 98%, or at least 99% identical to the heavy chain sequence of a co-receptor antibody. In some embodiments, the polynucleotide comprises a sequence that encodes the heavy chain sequence of a co-receptor antibody. In some embodiments, the polynucleotide comprises a sequence that encodes a light chain sequence at least 80%, at least 85%, at least 90%, at least 92%, at least 95%, at least 98%, or at least 99% identical to the light chain sequence of a co-receptor antibody. In some embodiments, the polynucleotide comprises a sequence that encodes the light chain sequence of a co-receptor antibody. In some embodiments, a first polynucleotide comprises a sequence that encodes the heavy chain sequence of a co-receptor antibody and a second polynucleotide comprises a sequence that encodes the light chain sequence of a co-receptor antibody.

In some embodiments, the co-receptor binding site antibody is 48d. In some embodiments, the polynucleotide comprises a sequence that encodes a heavy chain sequence at least 80%, at least 85%, at least 90%, at least 92%, at least 95%, at least 98%, or at least 99% identical to the heavy chain sequence of the 48d antibody. In some embodiments, the polynucleotide comprises a sequence that encodes the heavy chain sequence of the 48d antibody. In some embodiments, the polynucleotide comprises a sequence that encodes a light chain sequence at least 80%, at least 85%, at least 90%, at least 92%, at least 95%, at least 98%, or at least 99% identical to the light chain sequence of the 48d antibody. In some embodiments, the polynucleotide comprises a sequence that encodes the light chain sequence of the 48d antibody. In some embodiments, a third and fourth polypeptide each comprise a sequence that encodes an antigen at least 80%, at least 85%, at least 90%, at least 92%, at least 95%, at least 98%, or at least 99% identical to an HIV gp120, Core, or epitope thereof.

In some embodiments, a first polynucleotide comprises a sequence that encodes a heavy chain sequence at least 80%, at least 85%, at least 90%, at least 92%, at least 95%, at least 98%, or at least 99% identical to SEQ ID NO: 1 and a second polynucleotide comprises a sequence that encodes a light chain sequence at least 80%, at least 85%, at least 90%, at least 92%, at least 95%, at least 98%, or at least 99% identical to SEQ ID NO: 3. In some embodiments, a first polynucleotide comprises a sequence that encodes a heavy chain sequence at least 80%, at least 85%, at least 90%, at least 92%, at least 95%, at least 98%, or at least 99% identical to SEQ ID NO: 1 and a second polynucleotide comprises a sequence that encodes a light chain sequence at least 80%, at least 85%, at least 90%, at least 92%, at least 95%, at least 98%, or at least 99% identical to SEQ ID NO: 5. In some embodiments, a first polynucleotide comprises a sequence that encodes a heavy chain sequence at least 80%, at least 85%, at least 90%, at least 92%, at least 95%, at least 98%, or at least 99% identical to SEQ ID NO: 1 and a second polynucleotide comprises a sequence that encodes a light chain sequence at least 80%, at least 85%, at least 90%, at least 92%, at least 95%, at least 98%, or at least 99% identical to SEQ ID No: 7. In some embodiments, a first polynucleotide comprises a sequence that encodes a heavy chain sequence at least 80%, at least 85%, at least 90%, at least 92%, at least 95%, at least 98%, or at least 99% identical to SEQ ID NO: 1 and a second polynucleotide comprises a sequence that encodes a light chain sequence at least 80%, at least 85%, at least 90%, at least 92%, at least 95%, at least 98%, or at least 99% identical to SEQ ID NO: 9. In some embodiments, a first polynucleotide comprises a sequence that encodes a heavy chain sequence at least 80%, at least 85%, at least 90%, at least 92%, at least 95%, at least 98%, or at least 99% identical to SEQ ID NO: 1 and a second polynucleotide comprises a sequence that encodes a light chain sequence at least 80%, at least 85%, at least 90%, at least 92%, at least 95%, at least 98%, or at least 99% identical to SEQ ID NO11. In some embodiments, a third and fourth polypeptide each comprise a sequence that encodes an antigen sequence at least 80%, at least 85%, at least 90%, at least 92%, at least 95%, at least 98%, or at least 99% identical to SEQ ID NO: 13. In some embodiments, a third and fourth polypeptide each comprise a sequence that encodes an antigen sequence at least 80%, at least 85%, at least 90%, at least 92%, at least 95%, at least 98%, or at least 99% identical to SEQ ID NO: 15.

In some embodiments, a first polynucleotide comprises a sequence that encodes the heavy chain sequence of the 48d antibody and a second polynucleotide comprises a sequence that encodes the light chain sequence of the 48d antibody. In some embodiments, a third and fourth polypeptide each comprise a sequence that encodes an HIV gp120 antigen, Core, or epitope thereof.

In some embodiments, the polynucleotide comprises a sequence that encodes a heavy chain sequence comprising any one of SEQ ID NOs: 1, 17, 19, 25, 29, 33, 37, 41, 45, 49, 51, 63, and 67. In some embodiments, the polynucleotide comprises a sequence that encodes a light chain sequence comprising any one of SEQ ID NOs: 3, 5, 7, 9, 11, 21, 23, 27, 31, 35, 39, 43, 47, 61, 65, and 69. In some embodiments, a first polynucleotide comprises a sequence that encodes a heavy chain sequence comprising any one of SEQ ID NOs: 1, 17, 49, or 51 and a second polynucleotide comprises a sequence that encodes a light chain sequence comprising any one of SEQ ID NOs: 3, 5, 7, 9, 11, and 62. In some embodiments, a first polynucleotide comprises a sequence that encodes SEQ ID NO: 19 and a second polynucleotide comprises a sequence that encodes SEQ ID NO: 21. In some embodiments, a first polynucleotide comprises a sequence that encodes SEQ ID NO: 19 and a second polynucleotide comprises a sequence that encodes SEQ ID NO: 23. In some embodiments, a first polynucleotide comprises a sequence that encodes SEQ ID NO: 25 and a second polynucleotide comprises a sequence that encodes SEQ ID NO: 27. In some embodiments, a first polynucleotide comprises a sequence that encodes SEQ ID NO: 29 and a second polynucleotide comprises a sequence that encodes SEQ ID NO: 31. In some embodiments, a first polynucleotide comprises a sequence that encodes SEQ ID NO: 33 and a second polynucleotide comprises a sequence that encodes SEQ ID NO: 35. In some embodiments, a first polynucleotide comprises a sequence that encodes SEQ ID NO: 37 and a second polynucleotide comprises a sequence that encodes SEQ ID NO: 39. In some embodiments, a first polynucleotide comprises a sequence that encodes SEQ ID NO: 41 and a second polynucleotide comprises a sequence that encodes SEQ ID NO: 43. In some embodiments, a first polynucleotide comprises a sequence that encodes SEQ ID NO: 45 and a second polynucleotide comprises a sequence that encodes SEQ ID NO: 47. In some embodiments, a first polynucleotide comprises a sequence that encodes SEQ ID NO: 63 and a second polynucleotide comprises a sequence that encodes SEQ ID NO: 65. In some embodiments, a first polynucleotide comprises a sequence that encodes SEQ ID NO: 67 and a second polynucleotide comprises a sequence that encodes SEQ ID NO: 69. In some embodiments, a third and fourth polynucleotide each comprise a sequence that encodes SEQ ID NO: 13. In some embodiments, a third and fourth polynucleotide each comprise a sequence that encodes SEQ ID NO: 15.

In some embodiments, a first polynucleotide comprises a sequence that encodes a heavy chain sequence comprising SEQ ID NO: 1 and a second polynucleotide encodes a light chain sequence comprising SEQ ID NO: 5. In some embodiments, a first polynucleotide comprises a sequence that encodes a heavy chain sequence comprising SEQ ID NO: 1 and a second polynucleotide encodes a light chain sequence comprising SEQ ID No: 7. In some embodiments, a first polynucleotide comprises a sequence that encodes a heavy chain sequence comprising SEQ ID NO: 1 a second polynucleotide encodes a light chain sequence comprising 9. In some embodiments, a first polynucleotide comprises a sequence that encodes a heavy chain sequence comprising SEQ ID NO: 1 a second polynucleotide encodes a light chain sequence comprising SEQ ID NO: 11. In some embodiments, a third and fourth polynucleotide each comprise a sequence that encodes SEQ ID NO: 13. In some embodiments, a third and fourth polynucleotide each comprise a sequence that encodes SEQ ID NO: 15.

In some embodiments, the polynucleotides comprise a sequence at least 80%, at least 85%, at least 90%, at least 92%, at least 95%, at least 98%, or at least 99% identical at least one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 62, 64, 66, 68, and 70. In some embodiments, a first polynucleotide comprises a sequence at least 80%, at least 85%, at least 90%, at least 92%, at least 95%, at least 98%, or at least 99% identical to at least one of SEQ ID NOs: 2, 18, 20, 26, 30, 34, 38, 42, 46, 50, 52, 64, and 68, and a second polynucleotide comprises a sequence at least 80%, at least 85%, at least 90%, at least 92%, at least 95%, at least 98%, or at least 99% identical to at least one of SEQ ID NOs: 4, 6, 8, 10, 12, 22, 24, 28, 32, 36, 40, 44, 48, 62, 66, and 70.

In some embodiments, the polynucleotides comprise at least one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 62, 64, 66, 68, and 70. In some embodiments, a first polynucleotide comprises any one of SEQ ID NOs: 2, 18, 20, 26, 30, 34, 38, 42, 46, 50, 52, 64, and 68, and a second polynucleotide comprises any one of SEQ ID NOs: 4, 6, 8, 10, 12, 22, 24, 28, 32, 36, 40, 44, 48, 62, 66, and 70.

In some embodiments, a first polynucleotide comprises SEQ ID NO: 2 and a second polynucleotide comprises SEQ ID NO: 4. In some embodiments, a first polynucleotide comprises SEQ ID NO: 2 and a second polynucleotide comprises SEQ ID NO: 6. In some embodiments, a first polynucleotide comprises SEQ ID NO: 2 and a second polynucleotide comprises SEQ ID NO: 8. In some embodiments, a first polynucleotide comprises SEQ ID NO: 2 and a second polynucleotide comprises SEQ ID NO: 10. In some embodiments, a first polynucleotide comprises SEQ ID NO: 2 and a second polynucleotide comprises SEQ ID NO: 12. In some embodiments, a first polynucleotide comprises SEQ ID NO: 2 and a second polynucleotide comprises SEQ ID NO: 62. In some embodiments, a third and fourth polynucleotide each comprise SEQ ID NO: 14. In some embodiments, a third and fourth polynucleotide each comprise SEQ ID NO: 16.

In some embodiments, a first polynucleotide comprises SEQ ID NO: 18 and a second polynucleotide comprises SEQ ID NO: 4. In some embodiments, a first polynucleotide comprises SEQ ID NO: 18 and a second polynucleotide comprises SEQ ID NO: 6. In some embodiments, a first polynucleotide comprises SEQ ID NO: 18 and a second polynucleotide comprises SEQ ID NO: 8. In some embodiments, a first polynucleotide comprises SEQ ID NO: 18 and a second polynucleotide comprises SEQ ID NO: 10. In some embodiments, a first polynucleotide comprises SEQ ID NO: 18 and a second polynucleotide comprises SEQ ID NO: 12.

In some embodiments, a first polynucleotide comprises SEQ ID NO: 20 and a second polynucleotide comprises SEQ ID NO: 22. In some embodiments, a first polynucleotide comprises SEQ ID NO: 20 and a second polynucleotide comprises SEQ ID NO: 24. In some embodiments, a first polynucleotide comprises SEQ ID NO: 26 and a second polynucleotide comprises SEQ ID NO: 28. In some embodiments, a first polynucleotide comprises SEQ ID NO: 30 and a second polynucleotide comprises SEQ ID NO: 32. In some embodiments, a first polynucleotide comprises SEQ ID NO: 34 and a second polynucleotide comprises SEQ ID NO: 36. In some embodiments, a first polynucleotide comprises SEQ ID NO: 38 and a second polynucleotide comprises SEQ ID NO: 40. In some embodiments, a first polynucleotide comprises SEQ ID NO: 42 and a second polynucleotide comprises SEQ ID NO: 44. In some embodiments, a first polynucleotide comprises SEQ ID NO: 46 and a second polynucleotide comprises SEQ ID NO: 48. In some embodiments, a first polynucleotide comprises SEQ ID NO: 64 and a second polynucleotide comprises SEQ ID NO: 66. In some embodiments, a first polynucleotide comprises SEQ ID NO: 68 and a second polynucleotide comprises SEQ ID NO: 70. In some embodiments, a third and fourth polynucleotide each comprise SEQ ID NO: 14. In some embodiments, a third and fourth polynucleotide each comprise SEQ ID NO: 16.

The present disclosure also provides cells comprising at least one polynucleotide described herein. In some embodiments, the cell is a prokaryotic cell. In some embodiments, the cell is a eukaryotic cell. In some embodiments, the cell is a mammalian cell (e.g., a murine, bovine, simian, porcine, equine, ovine, or human cell). In some embodiments, the cell is a human cell.

Non-limiting examples of polynucleotide sequences that may encode divalent immunogen scaffolds are shown in Table 3 and SEQ ID NOs: 2, 4, 6, 8, 10, 12, 18, 20, 22, 24, 26, 28, 30, 32, 24, 36, 38, 40, 42, 44, 46, 48, 50, 52, 62, 64, 66, 68, and 70.

TABLE 3
Non-limiting examples of sequences comprised by polynucleotides encoding
divalent immunogen scaffolds

| | SEQ ID | |
|---|---|---|
| ID | NO. | Sequence |
| 48d | 2 | ATGGGATGGTCATGTATCATCCTTTTTCTAGTAGCAACTGCAACCGGTGTACA |
| antibody | | TTCCGAAGTTCAACTTGTGCAGTCAGGAGCTGAGGTCAAGAAACCCGGTGCT |
| HC | | ACAGTGAAGATTAGCTGCAAAGCAAGCGGCTATACCTTTTCAGACTTTTATAT |
| | | GTACTGGGTTCGCCAAGCCCCCGGCAAAGGTCTGGAATGGATGGGCCTGATT |
| | | GATCCAGAAGACGCCTGCACTATGTACGCAGAGAAATTCCGAGGTAGGGTTA |
| | | CTATCACTGCTGACACGTCCACCGATACAGGCTATCTAGAGCTTAGCTCTCTA |
| | | AGAAGTGAAGACACGGCAGTGTATTATTGTGCTGCCGACCCATGGGAACTCA |
| | | ATGCCTTTAACGTTTGGGGACAAGGGACACTTGTCAGCGTTTCAAGCGCCTCG |
| | | ACCAAGGGCCCATCGGTCTTCCCCCTGGCACCCTCCTCCAAGAGCACCTCTGG |
| | | GGGCACAGCGGCCCTGGGCTGCCTGGTCAAGGACTACTTCCCCGAACCGGTG |
| | | ACGGTGTCGTGGAACTCAGGCGCCCTGACCAGCGGCGTGCACACCTTCCCGG |
| | | CTGTCCTACAGTCCTCAGGACTCTACTCCCTCAGCAGCGTGGTGACCGTGCCC |
| | | TCCAGCAGCTTGGGCACCCAGACCTACATCTGCAACGTGAATCACAAGCCCA |
| | | GCAACACCAAGGTGGACAAGAAAGTTGAGCCCAAATCTTGTGACAAAACTCA |
| | | CACATGCCCACCGTGCCCAGCACCTAACGCTGCTGGTGGACCATCCGTCTTCA |
| | | TCTTCCCTCCAAAGATCAAGGATGTACTCATGATCTCCCTGAGCCCCATAGTC |
| | | ACATGTGTGGTGGTGGATGTGAGCGAGGATGACCCAGATGTCCAGATCAGCT |
| | | GGTTTGTGAACAACGTGGAAGTACACACAGCTCAGACACAAACCCATAGAGA |
| | | GGATTACAACAGTACTCTCCGGGTGGTCAGTGCCCTCCCCATCCAGCACCAGG |
| | | ACTGGATGAGTGGCAAGGAGTTCAAATGCAAGGTCAACAACAAAGACCTCGG |
| | | CGCGCCCATCGAGAGAACCATCTCAAAACCCAAAGGGTCAGTAAGAGCTCCA |
| | | CAGGTATATGTCTTGCCTCCACCAGAAGAAGAGATGACTAAGAAACAGGTCA |
| | | CTCTGACCTGCATGGTCACAGACTTCATGCCTGAAGACATTTACGTGGAGTGG |
| | | ACCAACAACGGGAAAACAGAGCTAAACTACAAGAACACTGAACCAGTCCTG |
| | | GACTCTGATGGTTCTTACTTCATGTACAGCAAGCTGAGAGTGGAAAAGAAGA |
| | | ACTGGGTGGAAAGAAATAGCTACTCCTGTTCAGTGGTCCACGAGGGTCTGCA |
| | | CAATCACCACACGACTAAGAGCTTCTCCCGGACTCCGGGTTGA |
| 48d | 4 | ATGGGATGGTCATGTATCATCCTTTTTCTAGTAGCAACTGCAACCGGTGTACA |
| antibody | | TTCAGATATTCAAATGACGCAAAGTCCTAGCAGTGTCTCTGCCAGTGTGGGTG |
| LC | | ACCGAGTCACAATCACCTGTAGAGCTTCACAGGATATTAGTACCTGGTTGGCT |
| | | TGGTATCAACAAAAGCCTGGAAAGGCCCCAAAACTCCTGATTTACGCCGCCT |
| | | CAACATTGCAATCAGGAGTGCCTTCTAGATTTAGTGGGAGCGGGTCTGGGAC |
| | | CGACTTCTCACTTACAATTAACAGCCTGCAGCCGGAAGATTTTGCCACCTATT |
| | | ATTGCCAACAAGCTAACTCTTTCTTCACGTTCGGGGGGGGAACTAAAGTTGAG |
| | | ATTAAACGTACGGTGGCTGCACCATCTGTCTTCATCTTCCCGCCATCTGATGA |
| | | GCAGTTGAAATCTGGAACTGCCTCTGTTGTGTGCCTGCTGAATAACTTCTACC |
| | | CCAGAGAAGCCAAAGTGCAGTGGAAGGTGGACAACGCCCTGCAGAGCGGAA |
| | | ACAGCCAGGAAAGCGTGACAGAGCAGGATTCCAAGGATTCCACATACAGCCT |
| | | GAGCAGCACACTGACACTGTCCAAGGCCGACTACGAGAAGCACAAGGTGTAC |
| | | GCCTGCGAAGTGACACACCAGGGACTGTCCTCCCCTGTGACAAAGAGCTTCA |
| | | ACAGAGGAGAATGCTGA |
| 48d | 6 | ATGGGATGGTCATGTATCATCCTTTTTCTAGTAGCAACTGCAACCGGTGTACA |
| antibody | | TTCAGATATTCAAATGACGCAAAGTCCTAGCAGTGTCTCTGCCAGTGTGGGTG |
| LC K126C | | ACCGAGTCACAATCACCTGTAGAGCTTCACAGGATATTAGTACCTGGTTGGCT |
| | | TGGTATCAACAAAAGCCTGGAAAGGCCCCAAAACTCCTGATTTACGCCGCCT |
| | | CAACATTGCAATCAGGAGTGCCTTCTAGATTTAGTGGGAGCGGGTCTGGGAC |
| | | CGACTTCTCACTTACAATTAACAGCCTGCAGCCGGAAGATTTTGCCACCTATT |
| | | ATTGCCAACAAGCTAACTCTTTCTTCACGTTCGGGGGGGGAACTAAAGTTGAG |
| | | ATTAAACGTACGGTGGCTGCACCATCTGTCTTCATCTTCCCGCCATCTGATGA |
| | | GCAGTTGTGCTCTGGAACTGCCTCTGTTGTGTGCCTGCTGAATAACTTCTACC |
| | | CCAGAGAAGCCAAAGTGCAGTGGAAGGTGGACAACGCCCTGCAGAGCGGAA |
| | | ACAGCCAGGAAAGCGTGACAGAGCAGGATTCCAAGGATTCCACATACAGCCT |
| | | GAGCAGCACACTGACACTGTCCAAGGCCGACTACGAGAAGCACAAGGTGTAC |
| | | GCCTGCGAAGTGACACACCAGGGACTGTCCTCCCCTGTGACAAAGAGCTTCA |
| | | ACAGAGGAGAATGCTGA |
| 48d | 8 | ATGGGATGGTCATGTATCATCCTTTTTCTAGTAGCAACTGCAACCGGTGTACA |
| antibody | | TTCAGATATTCAAATGACGCAAAGTCCTAGCAGTGTCTCTGCCAGTGTGGGTG |
| LC Loop1 | | ACCGAGTCACAATCACCTGTAGAGCTTCACAGGATATTAGTACCTGGTTGGCT |
| | | TGGTATCAACAAAAGCCTGGAAAGGCCCCAAAACTCCTGATTTACGCCGCCT |
| | | CAACATTGCAATCAGGAGTGCCTTCTAGATTTAGTGGGAGCGGGTCTGGGAC |
| | | CGACTTCTCACTTACAATTAACAGCCTGCAGCCGGAAGATTTTGCCACCTATT |
| | | ATTGCCAACAAGCTAACTCTTTCTTCACGTTCGGGGGGGGAACTAAAGTTGAG |
| | | ATTAAACGTACGGTGGCTGCACCATCTGTCTTCATCTTCCCGCCAAACGATGA |
| | | ACAGCTGCTGCCTTGCGCAGGGCCGAGCAACGCCTCTGTTGTGTGCCTGCTGA |
| | | ATAACTTCTACCCCAGAGAAGCCAAAGTGCAGTGGAAGGTGGACAACGCCCT |
| | | GCAGAGCGGAAACAGCCAGGAAAGCGTGACAGAGCAGGATTCCAAGGATTC |
| | | CACATACAGCCTGAGCAGCACACTGACACTGTCCAAGGCCGACTACGAGAAG |
| | | CACAAGGTGTACGCCTGCGAAGTGACACACCAGGGACTGTCCTCCCCTGTGA |
| | | CAAAGAGCTTCAACAGAGGAGAATGCTGA |
| 48d | 10 | ATGGGATGGTCATGTATCATCCTTTTTCTAGTAGCAACTGCAACCGGTGTACA |
| antibody | | TTCAGATATTCAAATGACGCAAAGTCCTAGCAGTGTCTCTGCCAGTGTGGGTG |
| LC Loop2 | | ACCGAGTCACAATCACCTGTAGAGCTTCACAGGATATTAGTACCTGGTTGGCT |
| | | TGGTATCAACAAAAGCCTGGAAAGGCCCCAAAACTCCTGATTTACGCCGCCT |
| | | CAACATTGCAATCAGGAGTGCCTTCTAGATTTAGTGGGAGCGGGTCTGGGAC |
| | | CGACTTCTCACTTACAATTAACAGCCTGCAGCCGGAAGATTTTGCCACCTATT |
| | | ATTGCCAACAAGCTAACTCTTTCTTCACGTTCGGGGGGGGAACTAAAGTTGAG |
| | | ATTAAACGTACGGTGGCTGCACCATCTGTCTTCATCTTCCCGCCAGCACCTCC |
| | | TGAGGGGTGCGGGAGCCCTACTAGAACTGTTGTGTGCCTGCTGAATAACTTCT |
| | | ACCCCAGAGAAGCCAAAGTGCAGTGGAAGGTGGACAACGCCCTGCAGAGCG |
| | | GAAACAGCCAGGAAAGCGTGACAGAGCAGGATTCCAAGGATTCCACATACA |
| | | GCCTGAGCAGCACACTGACACTGTCCAAGGCCGACTACGAGAAGCACAAGGT |
| | | GTACGCCTGCGAAGTGACACACCAGGGACTGTCCTCCCCTGTGACAAAGAGC |
| | | TTCAACAGAGGAGAATGCTGA |
| 48d | 12 | ATGGGATGGTCATGTATCATCCTTTTTCTAGTAGCAACTGCAACCGGTGTACA |
| antibody | | TTCAGATATTCAAATGACGCAAAGTCCTAGCAGTGTCTCTGCCAGTGTGGGTG |
| LC Loop3 | | ACCGAGTCACAATCACCTGTAGAGCTTCACAGGATATTAGTACCTGGTTGGCT |
| | | TGGTATCAACAAAAGCCTGGAAAGGCCCCAAAACTCCTGATTTACGCCGCCT |
| | | CAACATTGCAATCAGGAGTGCCTTCTAGATTTAGTGGGAGCGGGTCTGGGAC |
| | | CGACTTCTCACTTACAATTAACAGCCTGCAGCCGGAAGATTTTGCCACCTATT |
| | | ATTGCCAACAAGCTAACTCTTTCTTCACGTTCGGGGGGGGAACTAAAGTTGAG |
| | | ATTAAACGTACGGTGGCTGCACCATCTGTCTTCATCTTCAGAGCAGAGGAGG |
| | | GGTGCGGGGGGAACACTGTGTACCTGGTGTGCCTGCTGAATAACTTCTACCCC |
| | | AGAGAAGCCAAAGTGCAGTGGAAGGTGGACAACGCCCTGCAGAGCGGAAAC |
| | | AGCCAGGAAAGCGTGACAGAGCAGGATTCCAAGGATTCCACATACAGCCTGA |
| | | GCAGCACACTGACACTGTCCAAGGCCGACTACGAGAAGCACAAGGTGTACGC |
| | | CTGCGAAGTGACACACCAGGGACTGTCCTCCCCTGTGACAAAGAGCTTCAAC |
| | | AGAGGAGAATGCTGA |
| gp120.Core | 18 | ATGCCCATGGGTAGCCTGCAACCACTGGCCACCCTGTATCTGTTAGGGATGTT |
| 48D + 48D. | | GGTGGCCTCCGTGCTTGCAGATATCCGACAGGCCCATTGCAATGTATCTAAAG |
| Heavy_Fusion | | CCACCTGGAATGAGACACTGGGTAAAGTCGTTAAGCAGTTGCGGAAACACTT |
| | | TGGCAATAATACGATCATCCGGTTCGCTAACTCCTCCGGGGGAGACTTGGAG |
| | | GTCACGACCCATTCTTTCAATTGCGGCGGAGAATTTTTTTATTGCAACACCTC |
| | | AGGCCTGTTCAATTCTACCTGGATTTCAAATACTAGTGTGCAGGGATCTAACA |
| | | GTACCGGTTCAAACGACAGCATAACATTGCCCTGTCGGATTAAACAGTGTATC |
| | | AACATGTGGCAGCGCATAGGTCAGGCCATGTATGCCCCTCCTATTCAAGGAG |
| | | TGATACGATGCGTGTCCAACATAACAGGACTTATTCTCACAAGAGATGGAGG |
| | | ATCCACCAACTCTACGACTGAGACCTTTAGACCAGGAGGTGGAGACATGAGA |
| | | GATAACTGGAGGAGTGAGCTTTACAAATACAAGGTCGTAAAAATCGAGGGGG |
| | | GGGTGTGGAAAGATGCCGAGACTACGTTATTCTGCGCCTCAGACGCCAAGGC |
| | | CTATGAAACAGAGAAACACAACGTGTGGGCAACCCATGCATGTGTTCCGACC |
| | | GACCCCAATCCACAGGAGATCCACCTGGAAGGCGTCACAGAAGAATTCAACA |
| | | TGTGGAAGAACAATATGGTGGAGCAGATGCACACCGATATTATTAGTCTGTG |
| | | GGACCAATCACTGAAACCATGTGTCAAACTTACCGGCGGTAGCGCCATCACC |
| | | CAGGCATGTCCTAAAGTAAGTTTCGAGCCAATCCCCATCCATTATTGCGCACC |
| | | TGCCGGCTTCGCAATCCTGAAATGTAAGGATAAGAAGTTCAATGGCACAGGC |
| | | CCTTGTCCGAGCGTGAGCACCGTCCAATGCACACACGGAATTAAACCTGTAG |
| | | TCAGCACTCAGCTGCTGCTGAATGGCTCCCTGGCAGAAGAGGAAGTCATGAT |
| | | CCGCTCAGAAAACATCACAAATAACGCTAAAAACATTCTGGTTCAATTTAAC |
| | | ACGCCTGTCCAGATCAACTGTACCAGGCCGGGCAACGGAGGTGGTGGTGGCA |
| | | GTGGGGGGGGTTCAGGTGGCGGCTCCGAAGTTCAACTTGTGCAGTCAGGAGC |
| | | TGAGGTCAAGAAACCCGGTGCTACAGTGAAGATTAGCTGCAAAGCAAGCGGC |
| | | TATACCTTTTCAGACTTTTATATGTACTGGGTTCGCCAAGCCCCCGGCAAAGG |
| | | TCTGGAATGGATGGGCCTGATTGATCCAGAAGACGCCTGCACTATGTACGCA |
| | | GAGAAATTCCGAGGTAGGGTTACTATCACTGCTGACACGTCCACCGATACAG |
| | | GCTATCTAGAGCTTAGCTCTCTAAGAAGTGAAGACACGGCAGTGTATTATTGT |
| | | GCTGCCGACCCATGGGAACTCAATGCCTTTAACGTTTGGGGACAAGGGACAC |
| | | TTGTCAGCGTTTCAAGCGCCTCGACCAAGGGCCCATCGGTCTTCCCCCTGGCA |
| | | CCCTCCTCCAAGAGCACCTCTGGGGGCACAGCGGCCCTGGGCTGCCTGGTCA |
| | | AGGACTACTTCCCCGAACCGGTGACGGTGTCGTGGAACTCAGGCGCCCTGAC |
| | | CAGCGGCGTGCACACCTTCCCGGCTGTCCTACAGTCCTCAGGACTCTACTCCC |
| | | TCAGCAGCGTGGTGACCGTGCCCTCCAGCAGCTTGGGCACCCAGACCTACAT |
| | | CTGCAACGTGAATCACAAGCCCAGCAACACCAAGGTGGACAAGAAAGTTGA |
| | | GCCCAAATCTTGTGACAAAACTCACACATGCCCACCGTGCCCAGCACCTAAC |
| | | GCTGCTGGTGGACCATCCGTCTTCATCTTCCCTCCAAAGATCAAGGATGTACT |
| | | CATGATCTCCCTGAGCCCCATAGTCACATGTGTGGTGGTGGATGTGAGCGAG |
| | | GATGACCCAGATGTCCAGATCAGCTGGTTTGTGAACAACGTGGAAGTACACA |
| | | CAGCTCAGACACAAACCCATAGAGAGGATTACAACAGTACTCTCCGGGTGGT |
| | | CAGTGCCCTCCCCATCCAGCACCAGGACTGGATGAGTGGCAAGGAGTTCAAA |
| | | TGCAAGGTCAACAACAAAGACCTCGGCGCGCCCATCGAGAGAACCATCTCAA |
| | | AACCCAAAGGGTCAGTAAGAGCTCCACAGGTATATGTCTTGCCTCCACCAGA |
| | | AGAAGAGATGACTAAGAAACAGGTCACTCTGACCTGCATGGTCACAGACTTC |
| | | ATGCCTGAAGACATTTACGTGGAGTGGACCAACAACGGGAAAACAGAGCTAA |
| | | ACTACAAGAACACTGAACCAGTCCTGGACTCTGATGGTTCTTACTTCATGTAC |
| | | AGCAAGCTGAGAGTGGAAAAGAAGAACTGGGTGGAAAGAAATAGCTACTCC |
| | | TGTTCAGTGGTCCACGAGGGTCTGCACAATCACCACACGACTAAGAGCTTCTC |
| | | CCGGACTCCGGGTTGA |
| RFD1_Heavy | 20 | ATGGGATGGTCATGTATCATCCTTTTTCTAGTAGCAACTGCAACCGGTGTACA |
| | | TTCCGAAGTTCAACTTGTGCAGTCAGGAGCTGAGGTCAAGAAACCCGGTGCT |
| | | ACAGTGAAGATTAGCTGCAAAGCAAGCGGCTATACCTTTTCAGACTTTTATAT |
| | | GTACTGGGTTCGCCAAGCCCCCGGCAAAGGTCTGGAATGGATGGGCCTGATT |
| | | GATCCAGAAGACGCCTGCACTATGTACGCAGAGAAATTCCGAGGTAGGGTTA |
| | | CTATCACTGCTGACACGTCCACCGATACAGGCTATCTAGAGCTTAGCTCTCTA |
| | | AGAAGTGAAGACACGGCAGTGTATTATTGTGCTGCCGACCCATGGGAACTCA |
| | | ATGCCTTTAACGTTTGGGGACAAGGGACACTTGTCAGCGTTTCAAGCGCCTCG |
| | | ACCAAGGGCCCATCGGTCTTCCCCCTGGCACCCTCCTCCAAGAGCACCTCTGG |
| | | GGGCACAGCGGCCCTGGGCTGCCTGGTCAAGGACTACTTCCCCGAACCGGTG |
| | | ACGGTGTCGTGGAACTCAGGCGCCCTGACCAGCGGCGTGCACACCTTCCCGG |
| | | CTGTCCTACAGTCCTCAGGACTCTACTCCCTCAGCAGCGTGGTGACCGTGCCC |
| | | TCCAGCAGCTTGGGCACCCAGACCTACATCTGCAACGTGAATCACAAGCCCA |
| | | GCAACACCAAGGTGGACAAGAAAGTTACAGCTAATACAGAAGTGACCCCTGA |
| | | GAGCGTGCGGGCCGCACTGCGCGAGCTGTTCGGAGATGAGGAACTGCTGGCC |
| | | TGGGCCGCAGAGCTTCTGAGAGCCTGA |
| RFD1_Light | 22 | ATGGGATGGTCATGTATCATCCTTTTTCTAGTAGCAACTGCAACCGGTGTACA |
| | | TTCAGATATTCAAATGACGCAAAGTCCTAGCAGTGTCTCTGCCAGTGTGGGTG |
| | | ACCGAGTCACAATCACCTGTAGAGCTTCACAGGATATTAGTACCTGGTTGGCT |
| | | TGGTATCAACAAAAGCCTGGAAAGGCCCCAAAACTCCTGATTTACGCCGCCT |
| | 24 | CAACATTGCAATCAGGAGTGCCTTCTAGATTTAGTGGGAGCGGGTCTGGGAC |
| | | CGACTTCTCACTTACAATTAACAGCCTGCAGCCGGAAGATTTTGCCACCTATT |
| | | ATTGCCAACAAGCTAACTCTTTCTTCACGTTCGGGGGGGGAACTAAAGTTGAG |
| | | ATTAAACGTACGGTGGCTGCACCATCTGTCTTCATCTTCCCGCCATCTGATGA |
| | | GCAGTTGAAATCTGGAACTGCCTCTGTTGTGTGCCTGCTGAATAACTTCTACC |
| | | CCAGAGAAGCCAAAGTGCAGTGGAAGGTGGACAACGCCCTGCAGAGCGGAA |
| | | ACAGCCAGGAAAGCGTGACAGAGCAGGATTCCAAGGATTCCACATACAGCCT |
| | | GAGCAGCACACTGACACTGTCCAAGGCCGACTACGAGAAGCACAAGGTGTAC |
| | | GCCTGCGAAGTGACACACCAGGGACTGTCCTCCCCTGTGACAGTGTCCTACG |
| | | AGAAGCTTGGACCCGGTCTGTGGATCTACCAGACTATTCAGGATGGCAAGCT |
| | | CCTGTCAATTTCCACTAATGCAAAGCCCAGCACTCCACTGGAAGAGAAAGTG |
| | | CGAGCTGTCGCCATCAGGGAGAAGCTTATCGCTGAGAACGCCGCCTGA |
| RFD1_Light. | | ATGCCCATGGGCAGCTTACAGCCCCTTGCCACACTGTATCTCTTGGGGATGCT |
| gpFusion: | | TGTTGCCTCCGTACTCGCCAGCGCAATCACTCAGGCTTGTCCAAAGGTTTCCT |
| | | TTGAACCGATACCCATTCACTATTGTGCTCCCGCCGGCTTCGCCATCCTTAAG |
| | | TGTAAGGACAAGAAATTCAATGGCACTGGCCCGTGTCCCTCCGTGTCCACCGT |
| | | GCAGTGCACCCATGGCATAAAACCCGTGGTGAGCACTCAGCTGCTGCTGAAC |
| | | GGTTCATTGGCCGAGGAAGAAGTTATGATCAGATCTGAGAACATCACGAATA |
| | | ACGCTAAAAATATCCTGGTTCAGTTCAACACCCCAGTACAAATCAATTGTACT |
| | | AGACCAGGCAACGGCGGCGACATCCGCCAGGCTCATTGTAACGTCTCCAAGG |
| | | CAACTTGGAATGAAACACTCGGGAAAGTGGTGAAACAGCTGCGCAAGCATTT |
| | | TGGGAATAACACCATCATACGATTCGCCAATAGCAGCGGGGGAGACCTGGAG |
| | | GTTACCACTCACAGTTTCAACTGTGGAGGCGAATTTTTCTATTGTAACACATC |
| | | AGGACTGTTCAATTCTACATGGATAAGTAACACATCAGTCCAGGGGTCAAAC |
| | | AGTACCGGAAGTAATGACAGCATCACCCTGCCCTGTAGAATTAAGCAGTGCA |
| | | TTAACATGTGGCAGCGGATCGGCCAGGCTATGTACGCCCCTCCCATACAGGG |
| | | CGTTATCCGTTGCGTGAGCAATATTACAGGCTTGATTTTAACTAGAGACGGCG |
| | | GTTCCACAAATTCTACAACCGAAACCTTCAGACCCGGCGGGGGCGACATGCG |
| | | TGACAACTGGCGCTCAGAACTCTACAAATACAAAGTTGTGAAAATTGAGGGA |
| | | GGCGTATGGAAGGACGCTGAAACCACCCTGTTCTGCGCAAGCGACGCCAAGG |
| | | CATATGAAACTGAGAAACACAACGTATGGGCCACACACGCTTGTGTCCCCAC |
| | | AGATCCCAACCCCCAAGAGATTCACCTCGAGGGTGTTACAGAGGAATTCAAC |
| | | ATGTGGAAAAACAACATGGTCGAGCAGATGCACACAGACATCATATCCCTCT |
| | | GGGATCAAAGTCTGAAGCCCTGCGTGAAACTGACTGGCGGCGGTAGTGGGGG |
| | | TGGATCAGGCGGTGGTAGTGATATTCAAATGACGCAAAGTCCTAGCAGTGTC |
| | | TCTGCCAGTGTGGGTGACCGAGTCACAATCACCTGTAGAGCTTCACAGGATAT |
| | | TAGTACCTGGTTGGCTTGGTATCAACAAAAGCCTGGAAAGGCCCCAAAACTC |
| | | CTGATTTACGCCGCCTCAACATTGCAATCAGGAGTGCCTTCTAGATTTAGTGG |
| | | GAGCGGGTCTGGGACCGACTTCTCACTTACAATTAACAGCCTGCAGCCGGAA |
| | | GATTTTGCCACCTATTATTGCCAACAAGCTAACTCTTTCTTCACGTTCGGGGG |
| | | GGGAACTAAAGTTGAGATTAAACGTACGGTGGCTGCACCATCTGTCTTCATCT |
| | | TCCCGCCATCTGATGAGCAGTTGAAATCTGGAACTGCCTCTGTTGTGTGCCTG |
| | | CTGAATAACTTCTACCCCAGAGAAGCCAAAGTGCAGTGGAAGGTGGACAACG |
| | | CCCTGCAGAGCGGAAACAGCCAGGAAAGCGTGACAGAGCAGGATTCCAAGG |
| | | ATTCCACATACAGCCTGAGCAGCACACTGACACTGTCCAAGGCCGACTACGA |
| | | GAAGCACAAGGTGTACGCCTGCGAAGTGACACACCAGGGACTGTCCTCCCCT |
| | | GTGACAGTGTCCTACGAGAAGCTTGGACCCGGTCTGTGGATCTACCAGACTAT |
| | | TCAGGATGGCAAGCTCCTGTCAATTTCCACTAATGCAAAGCCCAGCACTCCAC |
| | | TGGAAGAGAAAGTGCGAGCTGTCGCCATCAGGGAGAAGCTTATCGCTGAGAA |
| | | CGCCGCCTGA |
| RFD2_Heavy | 26 | ATGGGATGGTCATGTATCATCCTTTTTCTAGTAGCAACTGCAACCGGTGTACA |
| | | TTCCGAAGTTCAACTTGTGCAGTCAGGAGCTGAGGTCAAGAAACCCGGTGCT |
| | | ACAGTGAAGATTAGCTGCAAAGCAAGCGGCTATACCTTTTCAGACTTTTATAT |
| | | GTACTGGGTTCGCCAAGCCCCCGGCAAAGGTCTGGAATGGATGGGCCTGATT |
| | | GATCCAGAAGACGCCTGCACTATGTACGCAGAGAAATTCCGAGGTAGGGTTA |
| | | CTATCACTGCTGACACGTCCACCGATACAGGCTATCTAGAGCTTAGCTCTCTA |
| | | AGAAGTGAAGACACGGCAGTGTATTATTGTGCTGCCGACCCATGGGAACTCA |
| | | ATGCCTTTAACGTTTGGGGACAAGGGACACTTGTCAGCGTTTCAAGCGCCTCG |
| | | ACCAAGGGCCCATCGGTCTTCCCCCTGGCACCCTCCTCCAAGAGCACCTCTGG |
| | | GGGCACAGCGGCCCTGGGCTGCCTGGTCAAGGACTACTTCCCCGAACCGGTG |
| | | ACGGTGTCGTGGAACTCAGGCGCCCTGACCAGCGGCGTGCACACCTTCCCGG |
| | | CTGTCCTACAGTCCTCAGGACTCTACTCCCTCAGCAGCGTGGTGACCGTGCCC |
| | | TCCAGCAGCTTGGGCACCCAGACCTACATCTGCAACGTGAATCACAAGCCCA |
| | | GCAACACCAAGGTGGACAAGAAAGTTTCTACTCCAGTCGAGGCAGTGCTTCC |
| | | TCCCGAGGCACTTGCTCATTTGAGAGCTTTCCTGGCTCTGCTTCCGCTGGAGG |
| | | CCGCTCTTGCCGTGGCTCACGCCGTGCGGCAGGCCCTGATCGCAGTCCAA |
| RFD2_Light | 28 | ATGGGATGGTCATGTATCATCCTTTTTCTAGTAGCAACTGCAACCGGTGTACA |
| | | TTCAGATATTCAAATGACGCAAAGTCCTAGCAGTGTCTCTGCCAGTGTGGGTG |
| | | ACCGAGTCACAATCACCTGTAGAGCTTCACAGGATATTAGTACCTGGTTGGCT |
| | | TGGTATCAACAAAAGCCTGGAAAGGCCCCAAAACTCCTGATTTACGCCGCCT |
| | | CAACATTGCAATCAGGAGTGCCTTCTAGATTTAGTGGGAGCGGGTCTGGGAC |
| | | CGACTTCTCACTTACAATTAACAGCCTGCAGCCGGAAGATTTTGCCACCTATT |
| | | ATTGCCAACAAGCTAACTCTTTCTTCACGTTCGGGGGGGGAACTAAAGTTGAG |
| | | ATTAAACGTACGGTGGCTGCACCATCTGTCTTCATCTTCCCGCCATCTGATGA |
| | | GCAGTTGAAATCTGGAACTGCCTCTGTTGTGTGCCTGCTGAATAACTTCTACC |
| | | CCAGAGAAGCCAAAGTGCAGTGGAAGGTGGACAACGCCCTGCAGAGCGGAA |
| | | ACAGCCAGGAAAGCGTGACAGAGCAGGATTCCAAGGATTCCACATACAGCCT |
| | | GAGCAGCACACTGACACTGTCCAAGGCCGACTACGAGAAGCACAAGGTGTAC |
| | | GCCTGCGAAGTGACACACCAGGGACTGTCCTCCCCTGTGACAGCCAGAATCC |
| | | TGAGGGTGAACGATGTAGTATTTACTTCTACCCAGACCGAAACATCTGGAGA |
| | | GTGGACTGTTAACTCTACACCTCTGGATCAGCTCCTGCTTGATATCTACTTGG |
| | | AGGCCCAGGCCGCC |
| RFD3_Heavy | 30 | ATGGGATGGTCATGTATCATCCTTTTTCTAGTAGCAACTGCAACCGGTGTACA |
| | | TTCCGAAGTTCAACTTGTGCAGTCAGGAGCTGAGGTCAAGAAACCCGGTGCT |
| | | ACAGTGAAGATTAGCTGCAAAGCAAGCGGCTATACCTTTTCAGACTTTTATAT |
| | | GTACTGGGTTCGCCAAGCCCCCGGCAAAGGTCTGGAATGGATGGGCCTGATT |
| | | GATCCAGAAGACGCCTGCACTATGTACGCAGAGAAATTCCGAGGTAGGGTTA |
| | | CTATCACTGCTGACACGTCCACCGATACAGGCTATCTAGAGCTTAGCTCTCTA |
| | | AGAAGTGAAGACACGGCAGTGTATTATTGTGCTGCCGACCCATGGGAACTCA |
| | | ATGCCTTTAACGTTTGGGGACAAGGGACACTTGTCAGCGTTTCAAGCGCCTCG |
| | | ACCAAGGGCCCATCGGTCTTCCCCCTGGCACCCTCCTCCAAGAGCACCTCTGG |
| | | GGGCACAGCGGCCCTGGGCTGCCTGGTCAAGGACTACTTCCCCGAACCGGTG |
| | | ACGGTGTCGTGGAACTCAGGCGCCCTGACCAGCGGCGTGCACACCTTCCCGG |
| | | CTGTCCTACAGTCCTCAGGACTCTACTCCCTCAGCAGCGTGGTGACCGTGCCC |
| | | TCCAGCAGCTTGGGCACCCAGACCTACATCTGCAACGTGAATCACAAGCCCA |
| | | GCAACACCAAGGTGGACAAGAAAGTTGAGCCTAAAAACTCTGTGACACTGAC |
| | | ATCCACGGCTGCCGACAGGGAGCTCGAGGAGGCATTGAGGAAACTGGCCCCC |
| | | GAGGCCAAGTTTGTATTGGTCGGAACCCTGCCCGATGGCTCTACCCTCGTTAT |
| | | TGTGTTTGTTTCC |
| RFD3_Light | 32 | ATGGGATGGTCATGTATCATCCTTTTTCTAGTAGCAACTGCAACCGGTGTACA |
| | | TTCAGATATTCAAATGACGCAAAGTCCTAGCAGTGTCTCTGCCAGTGTGGGTG |
| | | ACCGAGTCACAATCACCTGTAGAGCTTCACAGGATATTAGTACCTGGTTGGCT |
| | | TGGTATCAACAAAAGCCTGGAAAGGCCCCAAAACTCCTGATTTACGCCGCCT |
| | | CAACATTGCAATCAGGAGTGCCTTCTAGATTTAGTGGGAGCGGGTCTGGGAC |
| | | CGACTTCTCACTTACAATTAACAGCCTGCAGCCGGAAGATTTTGCCACCTATT |
| | | ATTGCCAACAAGCTAACTCTTTCTTCACGTTCGGGGGGGGAACTAAAGTTGAG |
| | | ATTAAACGTACGGTGGCTGCACCATCTGTCTTCATCTTCCCGCCATCTGATGA |
| | | GCAGTTGAAATCTGGAACTGCCTCTGTTGTGTGCCTGCTGAATAACTTCTACC |
| | | CCAGAGAAGCCAAAGTGCAGTGGAAGGTGGACAACGCCCTGCAGAGCGGAA |
| | | ACAGCCAGGAAAGCGTGACAGAGCAGGATTCCAAGGATTCCACATACAGCCT |
| | | GAGCAGCACACTGACACTGTCCAAGGCCGACTACGAGAAGCACAAGGTGTAC |
| | | GCCTGCGAAGTGACACACCAGGGACTGTCCTCCCCTGTGACAAAGAGCTTCA |
| | | ATAAATATCCAGGCGTGGTGGTTACCGAAACAGTCGATGAGGCAACCGGCAC |
| | | CACTACAATTACCCTCAACACTAAAAACTATAAGGCAGTTCGCCTGGCCATCA |
| | | AAATTGCCGAAGCCCTGCATAAACTGCGTCCGGGCTCCACCACCGTTGCCGTT |
| | | ATCAGT |
| RFD4_Heavy | 34 | ATGGGATGGTCATGTATCATCCTTTTTCTAGTAGCAACTGCAACCGGTGTACA |
| | | TTCCGAAGTTCAACTTGTGCAGTCAGGAGCTGAGGTCAAGAAACCCGGTGCT |
| | | ACAGTGAAGATTAGCTGCAAAGCAAGCGGCTATACCTTTTCAGACTTTTATAT |
| | | GTACTGGGTTCGCCAAGCCCCCGGCAAAGGTCTGGAATGGATGGGCCTGATT |
| | | GATCCAGAAGACGCCTGCACTATGTACGCAGAGAAATTCCGAGGTAGGGTTA |
| | | CTATCACTGCTGACACGTCCACCGATACAGGCTATCTAGAGCTTAGCTCTCTA |
| | | AGAAGTGAAGACACGGCAGTGTATTATTGTGCTGCCGACCCATGGGAACTCA |
| | | ATGCCTTTAACGTTTGGGGACAAGGGACACTTGTCAGCGTTTCAAGCGCCTCG |
| | | ACCAAGGGCCCATCGGTCTTCCCCCTGGCACCCTCCTCCAAGAGCACCTCTGG |
| | | GGGCACAGCGGCCCTGGGCTGCCTGGTCAAGGACTACTTCCCCGAACCGGTG |
| | | ACGGTGTCGTGGAACTCAGGCGCCCTGACCAGCGGCGTGCACACCTTCCCGG |
| | | CTGTCCTACAGTCCTCAGGACTCTACTCCCTCAGCAGCGTGGTGACCGTGCCC |
| | | TCCAGCAGCTTGGGCACCCAGACCTACATCTGCAACGTGAATCACAAGCCCA |
| | | GCAACACCAAGGTGGACAAGAAAGTTCCCACAAACAAAGTGGTTACTGTGAC |
| | | AATCGAGGTCAAGGAGGAGGAGCTTCCCGAGGACGTGTCTGTGAGGATTCGT |
| | | ATGGAGATCGATATTAGTGAGAAGACAGGAAAGGTTGAAATGCGAATTGAA |
| | | GTTAGCAAGAGC |
| RFD4_Light | 36 | ATGGGATGGTCATGTATCATCCTTTTTCTAGTAGCAACTGCAACCGGTGTACA |
| | | TTCAGATATTCAAATGACGCAAAGTCCTAGCAGTGTCTCTGCCAGTGTGGGTG |
| | | ACCGAGTCACAATCACCTGTAGAGCTTCACAGGATATTAGTACCTGGTTGGCT |
| | | TGGTATCAACAAAAGCCTGGAAAGGCCCCAAAACTCCTGATTTACGCCGCCT |
| | | CAACATTGCAATCAGGAGTGCCTTCTAGATTTAGTGGGAGCGGGTCTGGGAC |
| | | CGACTTCTCACTTACAATTAACAGCCTGCAGCCGGAAGATTTTGCCACCTATT |
| | | ATTGCCAACAAGCTAACTCTTTCTTCACGTTCGGGGGGGGAACTAAAGTTGAG |
| | | ATTAAACGTACGGTGGCTGCACCATCTGTCTTCATCTTCCCGCCATCTGATGA |
| | | GCAGTTGAAATCTGGAACTGCCTCTGTTGTGTGCCTGCTGAATAACTTCTACC |
| | | CCAGAGAAGCCAAAGTGCAGTGGAAGGTGGACAACGCCCTGCAGAGCGGAA |
| | | ACAGCCAGGAAAGCGTGACAGAGCAGGATTCCAAGGATTCCACATACAGCCT |
| | | GAGCAGCACACTGACACTGTCCAAGGCCGACTACGAGAAGCACAAGGTGTAC |
| | | GCCTGCGAAGTGACACACCAGGGACTGTCCTCCCCTGTGACAGTTACCATCCA |
| | | CAGGTTTAAGCCCAATAATGTGGTCACCATCGAGAAACTGCCCAGCGGGAAA |
| | | ATCAAAGTGACCCTGACAGCTGAAAACGTTGAAGAAGCATTGGTTAACGCCT |
| | | ACAGGCTGGCTCTTGAACTAGCAGAGGAG |
| RFD5_Heavy | 38 | ATGGGATGGTCATGTATCATCCTTTTTCTAGTAGCAACTGCAACCGGTGTACA |
| | | TTCCGAAGTTCAACTTGTGCAGTCAGGAGCTGAGGTCAAGAAACCCGGTGCT |
| | | ACAGTGAAGATTAGCTGCAAAGCAAGCGGCTATACCTTTTCAGACTTTTATAT |
| | | GTACTGGGTTCGCCAAGCCCCCGGCAAAGGTCTGGAATGGATGGGCCTGATT |
| | | GATCCAGAAGACGCCTGCACTATGTACGCAGAGAAATTCCGAGGTAGGGTTA |
| | | CTATCACTGCTGACACGTCCACCGATACAGGCTATCTAGAGCTTAGCTCTCTA |
| | | AGAAGTGAAGACACGGCAGTGTATTATTGTGCTGCCGACCCATGGGAACTCA |
| | | ATGCCTTTAACGTTTGGGGACAAGGGACACTTGTCAGCGTTTCAAGCGCCTCG |
| | | ACCAAGGGCCCATCGGTCTTCCCCCTGGCACCCTCCTCCAAGAGCACCTCTGG |
| | | GGGCACAGCGGCCCTGGGCTGCCTGGTCAAGGACTACTTCCCCGAACCGGTG |
| | | ACGGTGTCGTGGAACTCAGGCGCCCTGACCAGCGGCGTGCACACCTTCCCGG |
| | | CTGTCCTACAGTCCTCAGGACTCTACTCCCTCAGCAGCGTGGTGACCGTGCCC |
| | | TCCAGCAGCTTGGGCACCCAGACCTACATCTGCAACGTGAATCACAAGCCCA |
| | | GCAACACCAAGGTGGACAAGAAAGTTGAACCCAAGAACTGGGTGAAGGCCA |
| | | CTGTTAAAGTCGAGGACGGGGTGGTGGAAGCCGAGTTTGACTCCGGTGGGAG |
| | | TAAAAACATCATTGAGGGACTGGCCAATTCTATGAAACTGCTCGCTGAGGTG |
| | | GCTCTAGAGCTGCTCAGC |
| RFD5_Light | 40 | ATGGGATGGTCATGTATCATCCTTTTTCTAGTAGCAACTGCAACCGGTGTACA |
| | | TTCAGATATTCAAATGACGCAAAGTCCTAGCAGTGTCTCTGCCAGTGTGGGTG |
| | | ACCGAGTCACAATCACCTGTAGAGCTTCACAGGATATTAGTACCTGGTTGGCT |
| | | TGGTATCAACAAAAGCCTGGAAAGGCCCCAAAACTCCTGATTTACGCCGCCT |
| | | CAACATTGCAATCAGGAGTGCCTTCTAGATTTAGTGGGAGCGGGTCTGGGAC |
| | | CGACTTCTCACTTACAATTAACAGCCTGCAGCCGGAAGATTTTGCCACCTATT |
| | | ATTGCCAACAAGCTAACTCTTTCTTCACGTTCGGGGGGGGAACTAAAGTTGAG |
| | | ATTAAACGTACGGTGGCTGCACCATCTGTCTTCATCTTCCCGCCATCTGATGA |
| | | GCAGTTGAAATCTGGAACTGCCTCTGTTGTGTGCCTGCTGAATAACTTCTACC |
| | | CCAGAGAAGCCAAAGTGCAGTGGAAGGTGGACAACGCCCTGCAGAGCGGAA |
| | | ACAGCCAGGAAAGCGTGACAGAGCAGGATTCCAAGGATTCCACATACAGCCT |
| | | GAGCAGCACACTGACACTGTCCAAGGCCGACTACGAGAAGCACAAGGTGTAC |
| | | GCCTGCGAAGTGACACACCAGGGACTGTCCTCCCCTGTGACAAAGTCTTTTAA |
| | | TAGGCTAAGTTCTGGAACATGGGCCACCATCGAGAAGAAGGAAGATCCCGGC |
| | | GAGATGAACGTGACAGCCAACGTAGCAAACGTTATTAGCGCTAAGATCAAGG |
| | | TCGAGTTCAAG |
| RFD6_Hcavy | 42 | ATGGGATGGTCATGTATCATCCTTTTTCTAGTAGCAACTGCAACCGGTGTACA |
| | | TTCCGAAGTTCAACTTGTGCAGTCAGGAGCTGAGGTCAAGAAACCCGGTGCT |
| | | ACAGTGAAGATTAGCTGCAAAGCAAGCGGCTATACCTTTTCAGACTTTTATAT |
| | | GTACTGGGTTCGCCAAGCCCCCGGCAAAGGTCTGGAATGGATGGGCCTGATT |
| | | GATCCAGAAGACGCCTGCACTATGTACGCAGAGAAATTCCGAGGTAGGGTTA |
| | | CTATCACTGCTGACACGTCCACCGATACAGGCTATCTAGAGCTTAGCTCTCTA |
| | | AGAAGTGAAGACACGGCAGTGTATTATTGTGCTGCCGACCCATGGGAACTCA |
| | | ATGCCTTTAACGTTTGGGGACAAGGGACACTTGTCAGCGTTTCAAGCGCCTCG |
| | | ACCAAGGGCCCATCGGTCTTCCCCCTGGCACCCTCCTCCAAGAGCACCTCTGG |
| | | GGGCACAGCGGCCCTGGGCTGCCTGGTCAAGGACTACTTCCCCGAACCGGTG |
| | | ACGGTGTCGTGGAACTCAGGCGCCCTGACCAGCGGCGTGCACACCTTCCCGG |
| | | CTGTCCTACAGTCCTCAGGACTCTACTCCCTCAGCAGCGTGGTGACCGTGCCC |
| | | TCCAGCAGCTTGGGCACCCAGACCTACATCTGCAACGTGAATCACAAGCCCA |
| | | GCAACACCAAGGTGGACAAGAAAGTTGAGCCAAAAGAGGTGCTCAACCGCG |
| | | TGACCGTGAAAGTGCCCGCCGAATTGACTGAGGCCGAAATTGCCATGCTCAA |
| | | GACTCTCCTGAAAGCCCTCGGTGAGGCTATTGCTAAGGCCGCTAAA |
| RFD6_Light | 44 | ATGGGATGGTCATGTATCATCCTTTTTCTAGTAGCAACTGCAACCGGTGTACA |
| | | TTCAGATATTCAAATGACGCAAAGTCCTAGCAGTGTCTCTGCCAGTGTGGGTG |
| | | ACCGAGTCACAATCACCTGTAGAGCTTCACAGGATATTAGTACCTGGTTGGCT |
| | | TGGTATCAACAAAAGCCTGGAAAGGCCCCAAAACTCCTGATTTACGCCGCCT |
| | | CAACATTGCAATCAGGAGTGCCTTCTAGATTTAGTGGGAGCGGGTCTGGGAC |
| | | CGACTTCTCACTTACAATTAACAGCCTGCAGCCGGAAGATTTTGCCACCTATT |
| | | ATTGCCAACAAGCTAACTCTTTCTTCACGTTCGGGGGGGGAACTAAAGTTGAG |
| | | ATTAAACGTACGGTGGCTGCACCATCTGTCTTCATCTTCCCGCCATCTGATGA |
| | | GCAGTTGAAATCTGGAACTGCCTCTGTTGTGTGCCTGCTGAATAACTTCTACC |
| | | CCAGAGAAGCCAAAGTGCAGTGGAAGGTGGACAACGCCCTGCAGAGCGGAA |
| | | ACAGCCAGGAAAGCGTGACAGAGCAGGATTCCAAGGATTCCACATACAGCCT |
| | | GAGCAGCACACTGACACTGTCCAAGGCCGACTACGAGAAGCACAAGGTGTAC |
| | | GCCTGCGAAGTGACACACCAGGGACTGTCCTCCCCTGTGACAAAGTCCTTCA |
| | | ACAAGGAGACCGGACTCGAGACAGATGTGGATCCCAAAGATGCAGACACTCC |
| | | TGAGTACAAAAGGGTGACCGTCTATACTCCGCTGACCGCAGAGGATGTGGAA |
| | | CTGTTTAAGAAGCTTGCTGAGAAGCTGGCCGAAGCCATCTATGAAGCCGCCA |
| | | AAGAAAGG |
| RFD7_Heavy | 46 | ATGGGATGGTCATGTATCATCCTTTTTCTAGTAGCAACTGCAACCGGTGTACA |
| | | TTCCGAAGTTCAACTTGTGCAGTCAGGAGCTGAGGTCAAGAAACCCGGTGCT |
| | | ACAGTGAAGATTAGCTGCAAAGCAAGCGGCTATACCTTTTCAGACTTTTATAT |
| | | GTACTGGGTTCGCCAAGCCCCCGGCAAAGGTCTGGAATGGATGGGCCTGATT |
| | | GATCCAGAAGACGCCTGCACTATGTACGCAGAGAAATTCCGAGGTAGGGTTA |
| | | CTATCACTGCTGACACGTCCACCGATACAGGCTATCTAGAGCTTAGCTCTCTA |
| | | AGAAGTGAAGACACGGCAGTGTATTATTGTGCTGCCGACCCATGGGAACTCA |
| | | ATGCCTTTAACGTTTGGGGACAAGGGACACTTGTCAGCGTTTCAAGCGCCTCG |
| | | ACCAAGGGCCCATCGGTCTTCCCCCTGGCACCCTCCTCCAAGAGCACCTCTGG |
| | | GGGCACAGCGGCCCTGGGCTGCCTGGTCAAGGACTACTTCCCCGAACCGGTG |
| | | ACGGTGTCGTGGAACTCAGGCGCCCTGACCAGCGGCGTGCACACCTTCCCGG |
| | | CTGTCCTACAGTCCTCAGGACTCTACTCCCTCAGCAGCGTGGTGACCGTGCCC |
| | | TCCAGCAGCTTGGGCACCCAGACCTACATCTGCAACGTGAATCACAAGCCCA |
| | | GCAACACCAAGGTGGACAAGAAAGTTGAGCCCAAGAACGGCTTCAGTCTTTC |
| | | TGGCTCCCCTGAAGAGGTGGTGGCACAGGCACTGAAGCATCATACCGGAGAT |
| | | GAAAAGGTGGCCAAGATGGGCGTCGCACTAGTGAAGGAGCTGTTGGCT |
| RFD7_Light | 48 | ATGGGATGGTCATGTATCATCCTTTTTCTAGTAGCAACTGCAACCGGTGTACA |
| | | TTCAGATATTCAAATGACGCAAAGTCCTAGCAGTGTCTCTGCCAGTGTGGGTG |
| | | ACCGAGTCACAATCACCTGTAGAGCTTCACAGGATATTAGTACCTGGTTGGCT |
| | | TGGTATCAACAAAAGCCTGGAAAGGCCCCAAAACTCCTGATTTACGCCGCCT |
| | | CAACATTGCAATCAGGAGTGCCTTCTAGATTTAGTGGGAGCGGGTCTGGGAC |
| | | CGACTTCTCACTTACAATTAACAGCCTGCAGCCGGAAGATTTTGCCACCTATT |
| | | ATTGCCAACAAGCTAACTCTTTCTTCACGTTCGGGGGGGGAACTAAAGTTGAG |
| | | ATTAAACGTACGGTGGCTGCACCATCTGTCTTCATCTTCCCGCCATCTGATGA |
| | | GCAGTTGAAATCTGGAACTGCCTCTGTTGTGTGCCTGCTGAATAACTTCTACC |
| | | CCAGAGAAGCCAAAGTGCAGTGGAAGGTGGACAACGCCCTGCAGAGCGGAA |
| | | ACAGCCAGGAAAGCGTGACAGAGCAGGATTCCAAGGATTCCACATACAGCCT |
| | | GAGCAGCACACTGACACTGTCCAAGGCCGACTACGAGAAGCACAAGGTGTAC |
| | | GCCTGCGAAGTGACACACCAGGGACTGTCCTCCCCTGTGACAAAGAGTTTCA |
| | | ACCGGTATCCCAGCGGCGTGATTGTATACAGGACGACCTCCAACGGCTCCAG |
| | | CAGCTACGGCATTGATGCTAATCTGAAGACACTGGAAGCACTGCTGGAATTT |
| | | ATCGAGGCAGTGGTTGAGATCTACAAAGAAAGG |
| 48D H.C56D. | 50 | ATGCCCATGGGTAGCCTGCAACCACTGGCCACCCTGTATCTGTTAGGGATGTT |
| MouseIgG2a. | | GGTGGCCTCCGTGCTTGCAGATATCCGACAGGCCCATTGCAATGTATCTAAAG |
| Kde1.Spy: | | CCACCTGGAATGAGACACTGGGTAAAGTCGTTAAGCAGTTGCGGAAACACTT |
| | | TGGCAATAATACGATCATCCGGTTCGCTAACTCCTCCGGGGGAGACTTGGAG |
| | | GTCACGACCCATTCTTTCAATTGCGGCGGAGAATTTTTTTATTGCAACACCTC |
| | | AGGCCTGTTCAATTCTACCTGGATTTCAAATACTAGTGTGCAGGGATCTAACA |
| | | GTACCGGTTCAAACGACAGCATAACATTGCCCTGTCGGATTAAACAGTGTATC |
| | | AACATGTGGCAGCGCATAGGTCAGGCCATGTATGCCCCTCCTATTCAAGGAG |
| | | TGATACGATGCGTGTCCAACATAACAGGACTTATTCTCACAAGAGATGGAGG |
| | | ATCCACCAACTCTACGACTGAGACCTTTAGACCAGGAGGTGGAGACATGAGA |
| | | GATAACTGGAGGAGTGAGCTTTACAAATACAAGGTCGTAAAAATCGAGGGGG |
| | | GGGTGTGGAAAGATGCCGAGACTACGTTATTCTGCGCCTCAGACGCCAAGGC |
| | | CTATGAAACAGAGAAACACAACGTGTGGGCAACCCATGCATGTGTTCCGACC |
| | | GACCCCAATCCACAGGAGATCCACCTGGAAGGCGTCACAGAAGAATTCAACA |
| | | TGTGGAAGAACAATATGGTGGAGCAGATGCACACCGATATTATTAGTCTGTG |
| | | GGACCAATCACTGAAACCATGTGTCAAACTTACCGGCGGTAGCGCCATCACC |
| | | CAGGCATGTCCTAAAGTAAGTTTCGAGCCAATCCCCATCCATTATTGCGCACC |
| | | TGCCGGCTTCGCAATCCTGAAATGTAAGGATAAGAAGTTCAATGGCACAGGC |
| | | CCTTGTCCGAGCGTGAGCACCGTCCAATGCACACACGGAATTAAACCTGTAG |
| | | TCAGCACTCAGCTGCTGCTGAATGGCTCCCTGGCAGAAGAGGAAGTCATGAT |
| | | CCGCTCAGAAAACATCACAAATAACGCTAAAAACATTCTGGTTCAATTTAAC |
| | | ACGCCTGTCCAGATCAACTGTACCAGGCCGGGCAACGGAGGTGGTGGTGGCA |
| | | GTGGGGGGGGTTCAGGTGGCGGCTCCGAAGTTCAACTTGTGCAGTCAGGAGC |
| | | TGAGGTCAAGAAACCCGGTGCTACAGTGAAGATTAGCTGCAAAGCAAGCGGC |
| | | TATACCTTTTCAGACTTTTATATGTACTGGGTTCGCCAAGCCCCCGGCAAAGG |
| | | TCTGGAATGGATGGGCCTGATTGATCCAGAAGACGCCTGCACTATGTACGCA |
| | | GAGAAATTCCGAGGTAGGGTTACTATCACTGCTGACACGTCCACCGATACAG |
| | | GCTATCTAGAGCTTAGCTCTCTAAGAAGTGAAGACACGGCAGTGTATTATTGT |
| | | GCTGCCGACCCATGGGAACTCAATGCCTTTAACGTTTGGGGACAAGGGACAC |
| | | TTGTCAGCGTTTCAAGCGCCTCGACCAAGGGCCCATCGGTCTTCCCCCTGGCA |
| | | CCCTCCTCCAAGAGCACCTCTGGGGGCACAGCGGCCCTGGGCTGCCTGGTCA |
| | | AGGACTACTTCCCCGAACCGGTGACGGTGTCGTGGAACTCAGGCGCCCTGAC |
| | | CAGCGGCGTGCACACCTTCCCGGCTGTCCTACAGTCCTCAGGACTCTACTCCC |
| | | TCAGCAGCGTGGTGACCGTGCCCTCCAGCAGCTTGGGCACCCAGACCTACAT |
| | | CTGCAACGTGAATCACAAGCCCAGCAACACCAAGGTGGACAAGAAAGTTGA |
| | | GCCCAAATCTTGTGACAAAACTCACACATGCCCACCGTGCCCAGCACCTAAC |
| | | GCTGCTGGTGGACCATCCGTCTTCATCTTCCCTCCAAAGATCAAGGATGTACT |
| | | CATGATCTCCCTGAGCCCCATAGTCACATGTGTGGTGGTGGATGTGAGCGAG |
| | | GATGACCCAGATGTCCAGATCAGCTGGTTTGTGAACAACGTGGAAGTACACA |
| | | CAGCTCAGACACAAACCCATAGAGAGGATTACAACAGTACTCTCCGGGTGGT |
| | | CAGTGCCCTCCCCATCCAGCACCAGGACTGGATGAGTGGCAAGGAGTTCAAA |
| | | TGCAAGGTCAACAACAAAGACCTCGGCGCGCCCATCGAGAGAACCATCTCAA |
| | | AACCCAAAGGGTCAGTAAGAGCTCCACAGGTATATGTCTTGCCTCCACCAGA |
| | | AGAAGAGATGACTAAGAAACAGGTCACTCTGACCTGCATGGTCACAGACTTC |
| | | ATGCCTGAAGACATTTACGTGGAGTGGACCAACAACGGGAAAACAGAGCTAA |
| | | ACTACAAGAACACTGAACCAGTCCTGGACTCTGATGGTTCTTACTTCATGTAC |
| | | AGCAAGCTGAGAGTGGAAAAGAAGAACTGGGTGGAAAGAAATAGCTACTCC |
| | | TGTTCAGTGGTCCACGAGGGTCTGCACAATCACCACACGACTAAGAGCTTCTC |
| | | CCGGACTCCGGGTTGA |
| 48D H. | 52 | ATGCCCATGGGTAGCCTGCAACCACTGGCCACCCTGTATCTGTTAGGGATGTT |
| ShortUpperHinge. | | GGTGGCCTCCGTGCTTGCAGATATCCGACAGGCCCATTGCAATGTATCTAAAG |
| MouseIgG2a. | | CCACCTGGAATGAGACACTGGGTAAAGTCGTTAAGCAGTTGCGGAAACACTT |
| Kde1.Spy: | | TGGCAATAATACGATCATCCGGTTCGCTAACTCCTCCGGGGGAGACTTGGAG |
| | | GTCACGACCCATTCTTTCAATTGCGGCGGAGAATTTTTTTATTGCAACACCTC |
| | | AGGCCTGTTCAATTCTACCTGGATTTCAAATACTAGTGTGCAGGGATCTAACA |
| | | GTACCGGTTCAAACGACAGCATAACATTGCCCTGTCGGATTAAACAGTGTATC |
| | | AACATGTGGCAGCGCATAGGTCAGGCCATGTATGCCCCTCCTATTCAAGGAG |
| | | TGATACGATGCGTGTCCAACATAACAGGACTTATTCTCACAAGAGATGGAGG |
| | | ATCCACCAACTCTACGACTGAGACCTTTAGACCAGGAGGTGGAGACATGAGA |
| | | GATAACTGGAGGAGTGAGCTTTACAAATACAAGGTCGTAAAAATCGAGGGGG |
| | | GGGTGTGGAAAGATGCCGAGACTACGTTATTCTGCGCCTCAGACGCCAAGGC |
| | | CTATGAAACAGAGAAACACAACGTGTGGGCAACCCATGCATGTGTTCCGACC |
| | | GACCCCAATCCACAGGAGATCCACCTGGAAGGCGTCACAGAAGAATTCAACA |
| | | TGTGGAAGAACAATATGGTGGAGCAGATGCACACCGATATTATTAGTCTGTG |
| | | GGACCAATCACTGAAACCATGTGTCAAACTTACCGGCGGTAGCGCCATCACC |
| | | CAGGCATGTCCTAAAGTAAGTTTCGAGCCAATCCCCATCCATTATTGCGCACC |
| | | TGCCGGCTTCGCAATCCTGAAATGTAAGGATAAGAAGTTCAATGGCACAGGC |
| | | CCTTGTCCGAGCGTGAGCACCGTCCAATGCACACACGGAATTAAACCTGTAG |
| | | TCAGCACTCAGCTGCTGCTGAATGGCTCCCTGGCAGAAGAGGAAGTCATGAT |
| | | CCGCTCAGAAAACATCACAAATAACGCTAAAAACATTCTGGTTCAATTTAAC |
| | | ACGCCTGTCCAGATCAACTGTACCAGGCCGGGCAACGGAGGTGGTGGTGGCA |
| | | GTGGGGGGGGTTCAGGTGGCGGCTCCGAAGTTCAACTTGTGCAGTCAGGAGC |
| | | TGAGGTCAAGAAACCCGGTGCTACAGTGAAGATTAGCTGCAAAGCAAGCGGC |
| | | TATACCTTTTCAGACTTTTATATGTACTGGGTTCGCCAAGCCCCCGGCAAAGG |
| | | TCTGGAATGGATGGGCCTGATTGATCCAGAAGACGCCTGCACTATGTACGCA |
| | | GAGAAATTCCGAGGTAGGGTTACTATCACTGCTGACACGTCCACCGATACAG |
| | | GCTATCTAGAGCTTAGCTCTCTAAGAAGTGAAGACACGGCAGTGTATTATTGT |
| | | GCTGCCGACCCATGGGAACTCAATGCCTTTAACGTTTGGGGACAAGGGACAC |
| | | TTGTCAGCGTTTCAAGCGCCTCGACCAAGGGCCCATCGGTCTTCCCCCTGGCA |
| | | CCCTCCTCCAAGAGCACCTCTGGGGGCACAGCGGCCCTGGGCTGCCTGGTCA |
| | | AGGACTACTTCCCCGAACCGGTGACGGTGTCGTGGAACTCAGGCGCCCTGAC |
| | | CAGCGGCGTGCACACCTTCCCGGCTGTCCTACAGTCCTCAGGACTCTACTCCC |
| | | TCAGCAGCGTGGTGACCGTGCCCTCCAGCAGCTTGGGCACCCAGACCTACAT |
| | | CTGCAACGTGAATCACAAGCCCAGCAACACCAAGGTGGACAAGAAAGTTGA |
| | | GCCCAAATCTTGTGACAAAACTCACACATGCCCACCGTGCCCAGCACCTAAC |
| | | GCTGCTGGTGGACCATCCGTCTTCATCTTCCCTCCAAAGATCAAGGATGTACT |
| | | CATGATCTCCCTGAGCCCCATAGTCACATGTGTGGTGGTGGATGTGAGCGAG |
| | | GATGACCCAGATGTCCAGATCAGCTGGTTTGTGAACAACGTGGAAGTACACA |
| | | CAGCTCAGACACAAACCCATAGAGAGGATTACAACAGTACTCTCCGGGTGGT |
| | | CAGTGCCCTCCCCATCCAGCACCAGGACTGGATGAGTGGCAAGGAGTTCAAA |
| | | TGCAAGGTCAACAACAAAGACCTCGGCGCGCCCATCGAGAGAACCATCTCAA |
| | | AACCCAAAGGGTCAGTAAGAGCTCCACAGGTATATGTCTTGCCTCCACCAGA |
| | | AGAAGAGATGACTAAGAAACAGGTCACTCTGACCTGCATGGTCACAGACTTC |
| | | ATGCCTGAAGACATTTACGTGGAGTGGACCAACAACGGGAAAACAGAGCTAA |
| | | ACTACAAGAACACTGAACCAGTCCTGGACTCTGATGGTTCTTACTTCATGTAC |
| | | AGCAAGCTGAGAGTGGAAAAGAAGAACTGGGTGGAAAGAAATAGCTACTCC |
| | | TGTTCAGTGGTCCACGAGGGTCTGCACAATCACCACACGACTAAGAGCTTCTC |
| | | CCGGACTCCGGGTTGA |
| gp120.Core | 62 | ATGCCCATGGGCAGCTTACAGCCCCTTGCCACACTGTATCTCTTGGGGATGCT |
| 48D + 48D. | | TGTTGCCTCCGTACTCGCCAGCGCAATCACTCAGGCTTGTCCAAAGGTTTCCT |
| Light_Fusion | | TTGAACCGATACCCATTCACTATTGTGCTCCCGCCGGCTTCGCCATCCTTAAG |
| | | TGTAAGGACAAGAAATTCAATGGCACTGGCCCGTGTCCCTCCGTGTCCACCGT |
| | | GCAGTGCACCCATGGCATAAAACCCGTGGTGAGCACTCAGCTGCTGCTGAAC |
| | | GGTTCATTGGCCGAGGAAGAAGTTATGATCAGATCTGAGAACATCACGAATA |
| | | ACGCTAAAAATATCCTGGTTCAGTTCAACACCCCAGTACAAATCAATTGTACT |
| | | AGACCAGGCAACGGCGGCGACATCCGCCAGGCTCATTGTAACGTCTCCAAGG |
| | | CAACTTGGAATGAAACACTCGGGAAAGTGGTGAAACAGCTGCGCAAGCATTT |
| | | TGGGAATAACACCATCATACGATTCGCCAATAGCAGCGGGGGAGACCTGGAG |
| | | GTTACCACTCACAGTTTCAACTGTGGAGGCGAATTTTTCTATTGTAACACATC |
| | | AGGACTGTTCAATTCTACATGGATAAGTAACACATCAGTCCAGGGGTCAAAC |
| | | AGTACCGGAAGTAATGACAGCATCACCCTGCCCTGTAGAATTAAGCAGTGCA |
| | | TTAACATGTGGCAGCGGATCGGCCAGGCTATGTACGCCCCTCCCATACAGGG |
| | | CGTTATCCGTTGCGTGAGCAATATTACAGGCTTGATTTTAACTAGAGACGGCG |
| | | GTTCCACAAATTCTACAACCGAAACCTTCAGACCCGGCGGGGGCGACATGCG |
| | | TGACAACTGGCGCTCAGAACTCTACAAATACAAAGTTGTGAAAATTGAGGGA |
| | | GGCGTATGGAAGGACGCTGAAACCACCCTGTTCTGCGCAAGCGACGCCAAGG |
| | | CATATGAAACTGAGAAACACAACGTATGGGCCACACACGCTTGTGTCCCCAC |
| | | AGATCCCAACCCCCAAGAGATTCACCTCGAGGGTGTTACAGAGGAATTCAAC |
| | | ATGTGGAAAAACAACATGGTCGAGCAGATGCACACAGACATCATATCCCTCT |
| | | GGGATCAAAGTCTGAAGCCCTGCGTGAAACTGACTGGCGGCGGTAGTGGGGG |
| | | TGGATCAGGCGGTGGTAGTGATATTCAAATGACGCAAAGTCCTAGCAGTGTC |
| | | TCTGCCAGTGTGGGTGACCGAGTCACAATCACCTGTAGAGCTTCACAGGATAT |
| | | TAGTACCTGGTTGGCTTGGTATCAACAAAAGCCTGGAAAGGCCCCAAAACTC |
| | | CTGATTTACGCCGCCTCAACATTGCAATCAGGAGTGCCTTCTAGATTTAGTGG |
| | | GAGCGGGTCTGGGACCGACTTCTCACTTACAATTAACAGCCTGCAGCCGGAA |
| | | GATTTTGCCACCTATTATTGCCAACAAGCTAACTCTTTCTTCACGTTCGGGGG |
| | | GGGAACTAAAGTTGAGATTAAACGTACGGTGGCTGCACCATCTGTCTTCATCT |
| | | TCCCGCCATCTGATGAGCAGTTGAAATCTGGAACTGCCTCTGTTGTGTGCCTG |
| | | CTGAATAACTTCTACCCCAGAGAAGCCAAAGTGCAGTGGAAGGTGGACAACG |
| | | CCCTGCAGAGCGGAAACAGCCAGGAAAGCGTGACAGAGCAGGATTCCAAGG |
| | | ATTCCACATACAGCCTGAGCAGCACACTGACACTGTCCAAGGCCGACTACGA |
| | | GAAGCACAAGGTGTACGCCTGCGAAGTGACACACCAGGGACTGTCCTCCCCT |
| | | GTGACAAAGAGCTTCAACAGAGGAGAATGCTGA |
| RFD2_v2_ | 64 | ATGGGCTGGTCCTGCATTATCCTGTTCCTGGTAGCGACAGCTACAGGTGTCCA |
| Heavy | | CTCTGAAGTGCAGCTTGTCCAGAGTGGCGCCGAAGTTAAGAAACCCGGAGCC |
| | | ACTGTGAAGATATCTTGTAAAGCTAGCGGATACACTTTCAGTGATTTTTATAT |
| | | GTATTGGGTGAGGCAAGCCCCTGGTAAGGGCCTGGAGTGGATGGGCCTCATC |
| | | GACCCAGAAGACGCCTGCACCATGTACGCTGAGAAGTTCCGAGGACGCGTCA |
| | | CAATTACAGCAGATACATCTACGGATACGGGATACCTTGAACTTAGCTCCCTG |
| | | CGGTCTGAGGATACCGCCGTGTATTATTGTGCAGCTGACCCCTGGGAACTCAA |
| | | CGCTTTTAACGTATGGGGCCAAGGAACACTCGTATCTGTTAGCGGTGCCGCGG |
| | | TGACTGGGCCACGAGTTTTTCCCCTGAGCCCAGCTGGCTGTTCTACCACATCA |
| | | CCAACTACACTCCTGGGTTGCCTGGTCGACGACTACTACCCCCTGCCCGTTAC |
| | | TGTAACCTGGAATGGCGGCGCTCTGACATCCGGCGTAACAACTTTCCCCGCCG |
| | | TCCTGCGGCCTGACGGCTTGTACAGCCAGGTGTCCGTGCTGAGCGTCCCCTCT |
| | | AGTAGCTGTGGCACAGAGACCTATACCTGCAACGTGACTCATCCAGCCTCCA |
| | | ACACAACCGTTTCACAGCTGGTGCCCTGCCCAAGACAACTGGAATTGCCGGC |
| | | CGAGGCCCTGGCGCACCTCAGGGCATTCCTGGCCTTGCTTCCGCCAGCCGCTG |
| | | CCCTTGCAGTCGCACATGCGGTACTCCAGGCACTCGCAGCCGCTCTTTAA |
| RFD2_v2_ | 66 | ATGGGATGGTCATGTATTATCCTTTTCCTGGTAGCCACTGCCACAGGAGTCCA |
| Light | | CAGCGACATCCAGATGACCCAGTCCCCATCTTCCGTTTCTGCTAGTGTCGGAG |
| | | ATAGAGTGACCATCACCTGCAGGGCCTCACAGGATATAAGTACGTGGCTGGC |
| | | CTGGTACCAGCAGAAACCCGGGAAGGCCCCTAAATTGCTGATTTACGCCGCC |
| | | TCCACACTCCAATCTGGGGTGCCTAGCCGGTTTTCCGGCAGTGGCAGCGGCAC |
| | | GGATTTCAGTCTGACCATCAACTCACTACAGCCTGAGGACTTTGCCACATACT |
| | | ACTGCCAGCAGGCCAACAGCTTCTTCACCTTTGGCGGAGGCACAAAGGTTGA |
| | | GATCAAGCGCACCGTCGTCGCCCCTAGTGTGAGCATCTTCCCCCCTAGCCCTG |
| | | AGGAGCTGGCATCCGGCCGCGCAAGGGTCGTGTGTGTAGCTTCCTCCTTTTAC |
| | | CCCGAGTCTCTGGAAGTTACCTGGCTGGTGGACGGAGAGCCAGTGAGCGGAA |
| | | GGTCTTTCACATCTACTTCAGCCAAGAACCCAGCAGACAATACCTATTCACTG |
| | | GTCTCAACCTTGGATCTTTCTACAGAAGAGTATCTGGCCCATAGGCGCTTTGA |
| | | ATGTAGAGTGACACATTCAGGCCTGAGCAGTCCTGTCACAGCCAGCATACTG |
| | | AGGGTCGGGGACACCCTGTTCACTTCTGTTTCTTCTTCTGATGATGGAGTGTTT |
| | | ACAGTCACCGGGTCTCCTCTGATGCAGCTGGCTCTGGATTTGTACTTGGAGGC |
| | | TCAGGCCGCCTAG |
| RFD3_v2_ | 68 | ATGGGGTGGTCCTGCATTATTCTGTTTTTGGTGGCTACCGCCACTGGAGTGCA |
| Heavy | | CAGCGAAGTACAGCTTGTGCAAAGTGGTGCTGAGGTGAAAAAGCCAGGCGCA |
| | | ACCGTGAAAATCAGCTGTAAGGCGAGCGGCTATACCTTCAGCGATTTCTACAT |
| | | GTACTGGGTGCGGCAGGCACCTGGTAAAGGACTGGAGTGGATGGGTCTGATC |
| | | GATCCAGAGGACGCCTGTACTATGTACGCGGAAAAATTTAGGGGTAGAGTGA |
| | | CCATTACCGCAGATACCTCAACTGATACTGGCTATCTGGAACTTTCAAGTTTG |
| | | CGCAGCGAAGATACCGCTGTCTACTATTGTGCAGCTGACCCCTGGGAACTTAA |
| | | CGCATTCAACGTGTGGGGGCAGGGAACATTGGTCTCAGTCTCCGGCGCTCCC |
| | | GTGACCGGGCCCGAGGTGTTTCCACTGCCACCTATGAGCGTAGGTAGCGGTA |
| | | AGCCAAGCGCAACCCTGGGGTGCCTCGTTAAGAACTTTTATCCACTGCCTGTC |
| | | GAAGTTACCTGGAATGGCGGCGCTGTCACAGAAGGTGTGTATGTGTTCCCTCC |
| | | GGTATTGCGGCCAGATGGCAGATGGAGTCTCGTCAGCGTGCTGAGGGTTCCG |
| | | GAGTCCACTCTTGGAACGGAGACATACGTGTGTAACGTGCGACATCCTCCAG |
| | | CGAATGTTACAGTGAGCAAAGAAGTGAAGGCTCTACCAGGTGTAACTCTCAC |
| | | ACAGACCCCTGAGGATGCTGCACTGGAAGCTAAGTTGAGGAAGGCCTGCCCT |
| | | GAAGCAGAAGCAATTATAGTGGGGACATTGCCTAACGGCGCGACCCTGGTCT |
| | | TGGTGTTTGTGCCCTGA |
| RFD3_v2_ | 70 | ATGGGATGGTCTTGCATCATTCTGTTTCTGGTGGCTACCGCTACTGGGGTACA |
| Light | | CAGCGATATACAGATGACCCAATCTCCCAGTTCCGTGTCCGCCTCCGTCGGAG |
| | | ATCGTGTGACTATTACATGCCGCGCTAGCCAAGACATCAGTACATGGCTAGC |
| | | ATGGTACCAGCAGAAGCCCGGGAAAGCACCCAAGCTTCTGATCTATGCAGCA |
| | | TCCACCCTGCAGTCAGGAGTGCCCAGCCGCTTTTCCGGAAGCGGGAGTGGAA |
| | | CCGATTTTTCTCTTACGATCAACTCTCTACAGCCCGAAGACTTCGCCACCTATT |
| | | ATTGTCAGCAGGCGAACAGTTTTTTCACATTTGGTGGCGGCACTAAGGTGGAA |
| | | ATCAAACGTACAGTGGCTGCTCCGTCCGTGTTCATCTTCCCCCCATCAGATGA |
| | | GCAGCTGAAATCTGGCACCGCTAGCGTGGTCTGTCTTCTGAACAATTTCTACC |
| | | CTCGCGAGGCTAAAGTCCAGTGGAAAGTTGACAACGCTCTGCAAAGCGGTAA |
| | | TTCCCAGGAATCTGTTACAGAGCAGGACTCCAAGGATAGCACATATTCCCTG |
| | | AGTAGTACCCTGACACTCAGCAAGGCCGACTACGAGAAGCATAAGGTTTACG |
| | | CGTGTGAAGTAACCCACCAGGGTTTGAGTAGCCCCGTTACCAAAAGTTTCAAT |
| | | AAGTATCCGGGCGTGGTGGTTACTGAAACCGTGGACGAGGCAACAGGTACCA |
| | | CTACTATTACTCTTAACACGAAGAACTACAAGGCAGTCCGTCTCGCCATAAAG |
| | | ATAGCCGAGGCTCTGCATAAGCTCAGGCCAGGTTCTACTACTGTTGCTGTCAT |
| | | TTCTTGA |

### Methods of Designing a Divalent Immunogen

The present disclosure also provides methods of designing divalent immunogens described herein. In some embodiments, the method comprises first identifying a target bnAb. In other embodiments, the method comprises first positioning conserved epitopes of the antigen in such a way that the epitopes would engage both Fabs of a theoretical or hypothesized bnAb simultaneously and with 1:1 stoichiometry.

In some embodiments, the scaffold comprises a partially de novo engineered protein scaffold. Scaffolds comprising a partially de novo engineered protein scaffold may be based on a monomeric, dimeric, or multimeric protein. Design of such a scaffold based on a dimeric or multimeric protein comprises several steps.

First, a target bnAb is identified. Second, two antigens or epitopes are modeled such that the target broadly neutralizing antibody (bnAb) will engage them with both Fabs simultaneously. Third, a dimeric protein or multimeric is modeled (structure obtained from the Protein Data Bank or structure prediction software) wherein the dimeric or multimeric protein is centrally positioned with respect to the two antigens without spatial overlap. Portions of the dimeric or multimeric protein that may interfere with grafting of the modeled antigens onto it are removed, and/or the protein is extended as required to achieve optimal spacing of the antigens. Fourth, for a rotationally symmetrical model including the modeled antigens and the dimeric or multimeric protein, protein design tools are used to connect a monomer (of the dimeric or multimeric protein) to a single antigen/epitope within the model. For a non-symmetrical model, protein design tools are used to connect each monomer of the dimeric protein to each of the antigens/epitopes. In either case, it is critical to avoid spatial overlap between the partially de novo scaffold and modeled bnAb Fabs. Non-limiting examples of divalent immunogens designed by this method are shown in FIG. 16 and FIG. 17 and in SEQ ID NOs: 71 and 72.

Design of such a scaffold based on a monomeric protein comprises several steps. First, a target bnAb is identified. Second, two antigens or epitopes are modeled such that the target broadly neutralizing antibody (bnAb) will engage them with both Fabs simultaneously. Third, a monomeric scaffold is modeled near the two antigens or epitopes. Fourth, protein design tools are used to create peptide linkages between the antigens or epitopes and the monomeric scaffold, avoiding overlap between the partially de novo scaffold and modeled bnAb Fabs.

In other embodiments, the scaffold comprises a completely de novo engineered protein scaffold. Scaffolds comprising a completely de novo engineered protein scaffold may be based on a monomeric, dimeric, or multimeric protein. First, a target bnAb is identified. Second, two antigens or epitopes are modeled such that the target bnAb will engage them with both Fabs simultaneously. Third, protein design tools are used to generate a de novo scaffold linking the two antigens, avoiding spatial overlap with the modeled bnAb Fabs. This de novo scaffold may include a dimerization domain.

In designing a scaffold comprising either a partially or fully de novo engineered protein scaffold, there are several considerations. In some embodiments, an essential feature is limitation of rotational or translational freedom of the antigen/epitope in the model, such that the broadly neutralizing antibody has a rare ability of simultaneous Fab engagement, compared with other antibodies that bind to other epitopes or with different angles of approach.

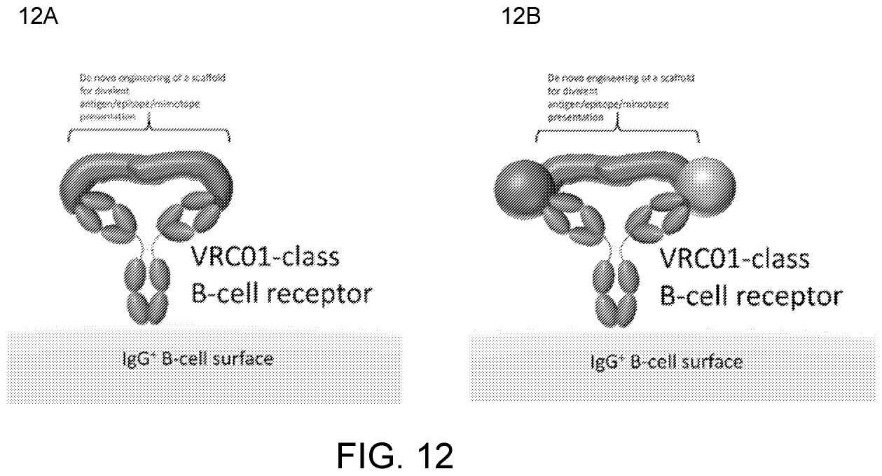

The antigen or epitope thereof may be incorporated through any method known in the art. The epitope may be grafted onto the scaffold (e.g., as in FIG. 12A) or alternatively, the entire antigen may be included (e.g., as in FIG. 12B) or a reduced (Core) antigen that includes the epitope may be included. In one non-limiting example, the antigen, the epitope, or the Core is linked to the scaffold-two separate proteins-using disulfide bonding or another covalent linkage. In a second non-limiting example, the antigen, the epitope, or the Core is fused to the scaffold such that it is one contiguous protein that folds into essentially the “scaffold” domain and the “antigen/epitope” domain, wherein these domains come together in the desired positioning upon completion of folding. In a third non-limiting example, the antigen, the epitope, or the Core is fully integrated into the scaffold such that there is less of a distinction between the “scaffold” domain and the “antigen/epitope” domain. Optionally, it may be closer to an integrated scaffold+epitope protein.

Divalent immunogen designs may be optimized by any method known in the art. In a non-limiting example, scaffold designs are generated using AI-based protein design tools. In a second, non-mutually-exclusive example, linkages of the scaffold to the antigens, Cores, or epitopes of interest are generated using AI-based protein design tools. Using this approach can achieve multiple important design goals, including:
Optimized positioning and idealized geometry of the two copies of the antigen/epitope for high avidity engagement with the target bnAb;
Rigid structure which increases the specificity of the divalent binding to the target bnAb—the “selective avidity effect”;
Possible incorporation of linear epitopes or other desirable features into the design;
Addition of any tags or domains required for multimerization of the divalent antigen;
Minimized off-target immunogenicity;
Optimized expression of the protein; and/or
Optimized purification method.

The present disclosure also provides methods that comprise first positioning conserved epitopes of the antigen in such a way that the epitopes would engage both Fabs of a theoretical or hypothesized bnAb simultaneously and with 1:1 stoichiometry. In some embodiments, the method comprises first identifying a “theoretical” or “hypothesized” template bnAb that would engage conserved epitopes at a particular angle of approach. In some embodiments, a divalent immunogen modeled thereon could elicit previously undiscovered and uncharacterized bnAbs with similar epitopes and angles of approach as the template bnAb.

First, the conserved epitopes of the antigen are positioned in such a way that they would engage both Fabs of such a theoretical or hypothesized bnAb simultaneously and with 1:1 stoichiometry. In some embodiments, the positioning of the epitopes provides rotational and positional flexibility. In some embodiments, the epitopes are about 3 to about 20 nm apart (i.e., have an epitopic distance of about 3 to about 20 nm). In some embodiments, the epitopes are about 7 to about 19 nm apart (i.e., have an epitopic distance of about 7 to about 19 nm). In some embodiments, the epitopes are about 13 to about 16 nm apart (i.e., have an epitopic distance of about 13 to about 16 nm). In some embodiments, the epitopes are positioned such that the two Fab domains of a bound BCR assume an angle of about 20 to about 150 degrees. In some embodiments, the epitopes are positioned such that the two Fab domains of a bound BCR assume an angle of about 90 to about 140 degrees. In some embodiments, the epitopes are positioned such that the two Fab domains of a bound BCR assume an angle of about 120 degrees.

This would be an alternative way of completing “Step 2” in the methods to design a divalent immunogen listed previously. Subsequent steps of completing the design would remain the same.

### Methods of Identifying Antibodies for Divalent Immunogens

The present disclosure also provides methods of identifying antibodies with preferred characteristics for divalent immunogens, comprising: (a) providing a divalent immunogen comprising a known broadly neutralizing antibody and an antigen or epitope thereof; (b) administering the divalent immunogen to a subject, optionally a mouse; (c) isolating B cells from the subject; (d) screening the isolated B cells for ability to bind to the divalent immunogen in high avidity, bidentate fashion; and (e) sequencing the complementarity determining regions of B cells that bind as in (d).

In some embodiments, the method comprises designing and providing an immune complex comprising a known broadly neutralizing antibody as a scaffold, and two antigens or epitopes thereof. In some embodiments, the method comprises administering the complex to an animal. In some embodiments, the complex is non-covalently or covalently cross-linked via chemical crosslinkers, engineered disulfide bonds, or expression of antibody-antigen chimeras. In some embodiments, the method activates/selects B cells whose receptors bind to the complex with high avidity. In some embodiments, the B cells undergo/continue affinity maturation.

In some embodiments, the B cells are isolated from the animal and screened for their ability to bind to the broadly neutralizing antibody-antigen complex in high avidity, bidentate fashion. In some embodiments, fluorescence-activated cell-sorting is used to isolate B-cells that preferentially bind to fluorophore-labeled intact broadly neutralizing antibody-antigen complex over a differentially labeled, fragmented bnAb-antigen complex. In some embodiments, the complementarity determining regions of these B cells will be sequenced, cloned and expressed to confirm the interaction using biochemical assays.

In some embodiments, once isolated from B-cells, the ideal antibody can be formulated into a divalent immunogen vaccine by complexation with the antigen. In some embodiments, the complexation may be non-covalent, or covalent in the form of chemical crosslinking, disulfide bond engineering or chimeric protein expression. In some embodiments, further mutations/chemical alterations to the antibody can be performed to lock the Fabs in a conformation that allows minimal entropic penalty in forming the ring-like structure with the BCR. In some embodiments, these modifications will also promote immune focusing by limiting the number of epitopes compatible with bidentate binding. In some embodiments, all or parts of the antibody may be “humanized” in order to minimize immunogenicity of the antibody alone. In some embodiments, the immune complex vaccine may be beneficially used to activate naïve B cells or may be used for further differentiation toward the desired specificity/breadth of neutralization of antibodies from B-cells already undergoing affinity maturation.

### Vaccines

The present disclosure provides vaccines comprising at least one of the divalent immunogens described herein. In some embodiments, the vaccine comprises or consists of at least one multimer of a divalent immunogen described herein. In some embodiments, the vaccine comprises or consists of a pharmaceutical composition described herein. In some embodiments, the vaccine is administered to a subject. A subject may be any mammal, including non-human primate and human subjects. Non-limiting examples of a subject are a mouse and a human.

### Methods of Generating an Immune Response

The present disclosure also provides methods of generating an immune response in a subject. In some embodiments, these methods comprise administering at least one divalent immunogen, at least one multimer, or at least one pharmaceutical composition disclosed herein, to a subject. The subject may be any mammal, including non-human primate and human subjects. In some embodiments, the subject is a mouse. In some embodiments, the subject is a human.

The divalent immunogens and/or pharmaceutical compositions disclosed herein can be administered to a subject by any mode of delivery, including, for example, by parenteral injection (e.g. subcutaneously, intraperitoneally, intravenously, intramuscularly, or to the interstitial space of a tissue), or by rectal, oral (e.g. tablet, spray), vaginal, topical, transdermal (e.g. see WO99/27961) or transcutaneous (e.g. see WO02/074244 and WO02/064162), intranasal (e.g. see WO03/028760), ocular, aural, pulmonary or other mucosal administration and/or inhalation of powder compositions. Multiple doses can be administered by the same or different routes.

The divalent immunogens, multimers, and/or pharmaceutical compositions disclosed herein can be administered prior to, concurrent with, or subsequent to delivery of other vaccines. Dosage with the divalent immunogens and pharmaceutical compositions may be a single dose schedule or a multiple dose schedule. A multiple dose schedule is one in which a course of vaccination may comprise 1-5 separate doses, in which a first “prime” dose is followed by other doses given at subsequent time intervals, chosen to maintain and/or reinforce the immune response. Non-limiting examples of time intervals between doses include 2 weeks, 3 weeks, 4 weeks, 6 weeks, 8 weeks, 3 months, 4 months, or 6 months. The dosage regimen will also, at least in part, be determined by the potency of the modality, the vaccine delivery employed, the need of the subject and be dependent on the judgment of the practitioner.

In some embodiments, the divalent immunogens and pharmaceutical compositions may be administered as “boost” vaccine dose after administration of a “prime” vaccine dose. The “prime” dose may be a different vaccine or may be a dose of a divalent immunogen or pharmaceutical composition of the present application. In other embodiments, the divalent immunogens and pharmaceutical compositions may be administered as a “prime” vaccine. One or more additional doses of a divalent immunogen or pharmaceutical composition of the present application, or of a different vaccine, may follow administration of the divalent immunogen and pharmaceutical composition. Also, the site of divalent immunogens or pharmaceutical composition administration may be the same or different as other vaccine compositions that are being administered.

In some embodiments, the divalent immunogen, multimer, pharmaceutical composition, or vaccine disclosed herein is formulated in an effective amount to produce an antigen specific immune response in a subject (e.g., production of antibodies specific to a specific antigen). “An effective amount” is a dose of the divalent immunogen, multimer, or pharmaceutical composition sufficient to produce an antigen-specific immune response.

In some embodiments, the immune response to a divalent immunogen, multimer, or pharmaceutical composition disclosed herein is the development in a subject of a humoral and/or a cellular immune response to an antigen present in the administered divalent immunogen, multimer, or pharmaceutical composition.

In some embodiments, the immune response results in an increased level (titer) of a certain antibody that binds an antigen or epitope thereof present in the divalent immunogen, multimer, or pharmaceutical composition that was administered. In some embodiments, the certain antibody is a neutralizing antibody. In some embodiments, the neutralizing antibody is a bnAb (e.g., a preferred bnAb).

In some embodiments, the immune response results in an increased number of B cells expressing a BCR that binds an antigen or epitope thereof present in the divalent immunogen, multimer, or pharmaceutical composition that was administered. In some embodiments, the BCR binds the divalent immunogen divalently with increased avidity. In some embodiments, the BCR binds both antigens of the divalent immunogen simultaneously with 1:1 stoichiometry. In some embodiments, the BCR binds both antigens of the divalent immunogen simultaneously, wherein the two Fab domains of the bound BCR assume an angle (Fab-Fab angle) disclosed herein. In some embodiments, the immune response results in an increased number of B cells expressing a certain (e.g., a preferred) BCR. In some embodiments, the immune response results in an increased number of B cells expressing a VRC01-class BCR.

An immune response generated by administering at least one divalent immunogen, multimer, and/or pharmaceutical composition disclosed herein may be measured by any suitable method known in the art. In some embodiments, the immune response is assessed by determining the titer of a certain bnAb (e.g., a preferred bnAb) in the subject. In other embodiments, the immune response is assessed by measuring the quantity and/or characteristics of B cells expressing a certain BCR (e.g., a preferred BCR) in the subject. In other embodiments, the ability of serum or antibody from an immunized subject is tested for its ability to neutralize viral uptake.

Measurement of the antibody titer, B cells, and/or neutralizing capacity may be quantified with respect to a control. In some embodiments, the antibody titer, B cell quantity/characteristics, and/or neutralizing capacity may be measured and compared to level(s) in the same subject prior to administering the divalent immunogen, multimer, or pharmaceutical composition. In some embodiments, the antibody titer, B cell quantity/characteristics, and/or neutralizing capacity may be measured and compared to level(s) in subjects administered a control substance (e.g., a placebo).

An antibody titer is a measurement of the amount of antibodies within a subject, for example, antibodies that are specific to a certain antigen or epitope of an antigen. In some embodiments, antibody titer is expressed as the inverse of the greatest dilution that provides a positive result. A non-limiting example of an assay for determining antibody titers is enzyme-linked immunosorbent assay (ELISA).

The quantity and/or characteristics of B cells expressing a certain (e.g., preferred) BCR may be assessed by metrics including, but not limited to, the number of B cells expressing a certain BCR, the number of B cells that recognize a certain antigen and/or divalent immunogen (e.g., through use of a specific probe), and surface markers of the B cells that indicate phenotype (e.g, activation or memory). A non-limiting example of an assay for measuring the quantity and/or characteristics of B cells is flow cytometry. BCRs may also be sequenced, to measure the BCR repertoire and the relative number of B cells expressing a certain BCR (e.g., a preferred BCR).

Neutralizing capacity of serum or antibody from subjects administered at least one divalent immunogen, multimer, or pharmaceutical composition disclosed herein may be measured by, e.g, a neutralization assay. Neutralization of different viral strains may be measured to assess breadth of neutralization.

### Methods of Forming Ring Structure

The present disclosure also provides methods of forming a ring structure comprising a divalent immunogen disclosed herein and a BCR or antibody. In some embodiments, the BCR is bound to both antigens or epitopes thereof with 1:1 stoichiometry. In some embodiments, the ring structure is preferentially formed by a certain (e.g., preferred) BCR or antibody. In some embodiments, the certain BCR or antibody is a broadly neutralizing BCR or antibody.

In some embodiments, the BCR or antibody is in a relaxed state. In some embodiments, the divalent immunogen comprises a specific antigenic spacing that allows the BCR and/or antibody to bind with a Fab-Fab angle of about 20 degrees to about 150 degrees. In some embodiments, the Fab-Fab angle is about 90 degrees to about 140 degrees. In some embodiments, the Fab-Fab angle is about 120 degrees.

In some embodiments, the ring structure is formed in a subject. In some embodiments, subject is a human.

1. A divalent immunogen comprising:
(a) a scaffold, and
(b) two antigens or epitopes thereof;
wherein each antigen or epitope thereof is covalently linked to the scaffold.

2. The divalent immunogen of claim 1, wherein only a fraction of antigen-reactive B-cell receptors are able to bind to both antigens or epitopes thereof simultaneously and with 1:1 stoichiometry.

3. The divalent immunogen of claim 1 or claim 2, wherein the scaffold is derived from an antibody.

4. The divalent immunogen of claim 1 or claim 2, where the scaffold is derived from a dimeric protein or a multimeric protein.

5. The divalent immunogen of claim 1 or claim 2, where the scaffold is derived from a monomeric protein.

6. The divalent immunogen of claim 1 or claim 2, wherein each antigen or epitope thereof is expressed as a fusion polypeptide comprising at least a part or fragment of the scaffold.

7. The divalent immunogen of claim 1 or claim 2, where the scaffold is a nucleic acid nanostructure.

8. The divalent immunogen of any one of claims 1-3, wherein the scaffold comprises an antibody or fragment or variant thereof.

9. The divalent complex of claim 8, wherein the scaffold comprises two Fab fragments or variants thereof.

10. The divalent immunogen of any one of claims 1-9, wherein the covalent linkage is a disulfide bond.

11. The divalent immunogen of any one of claims 1-9, wherein the covalent linkage is a chemical crosslinker, a protein crosslinker, or a nucleic acid crosslinker.

12. The divalent immunogen of any one of claims 9-11, wherein each Fab fragment comprises at least a first engineered Fab mutation that enables formation of the disulfide bond between the Fab fragment and the antigen or epitope thereof.

13. The divalent immunogen of any one of claims 9-12, wherein each Fab fragment comprises at least a second engineered Fab mutation that enables Fab-Fab crosslinking, wherein the second engineered Fab mutation is in the heavy chain or the light chain of each of the two Fab fragments.

14. The divalent immunogen of any one of claims 9-13, wherein the second engineered Fab mutation places the antigens or epitopes thereof in a more favorable orientation for divalent binding of at least one antibody.

15. The divalent immunogen of any one of claims 9-14, wherein the divalent immunogen comprises an engineered dimerization interface comprising C-terminal extension of the Fab heavy and/or light chains, optionally wherein the engineered dimerization interface places the antigens or epitopes thereof in a more favorable orientation for divalent binding of at least one B-cell receptor and/or antibody.

16. The divalent immunogen of any one of claims 1-15, wherein the divalent immunogen comprises a linker between the two antigens or epitopes thereof, optionally wherein the linker places the antigens or epitopes thereof in a more favorable orientation for divalent binding of at least one B-cell receptor and/or antibody.

17. The divalent immunogen of any one of claims 1-16, wherein the first antigen or epitope thereof is at least 70%, at least 80%, at least 90%, at least 95%, at least 98%, or at least 99% identical to the second antigen or epitope thereof.

18. The divalent immunogen of claim 17, wherein the first antigen or epitope thereof is 100% identical to the second antigen or epitope thereof.

19. The divalent immunogen of any one of claims 1-18, wherein each antigen or epitope thereof comprises a first engineered antigen mutation or a first engineered epitope mutation that enables formation of a disulfide bond between the scaffold and the antigen or epitope thereof.

20. The divalent immunogen of any one of claims 9-19, wherein the divalent immunogen additionally comprises an Fc domain.

21. The divalent immunogen of claim 20, wherein the Fc is a mouse Fc, optionally a mouse IgG2a Fc.

22. The divalent immunogen of claim 20, wherein the Fc is a human Fc, optionally a human IgG1 Fc.

23. The divalent immunogen of any one of claims 20-22, wherein the divalent immunogen comprises an engineered disulfide bond between the Fab and the Fc, optionally wherein the engineered disulfide bond places the antigens or epitopes thereof in a more favorable orientation for divalent binding of at least one B-cell receptor and/or antibody.

24. The divalent immunogen of any one of claims 20-23, wherein the divalent immunogen comprises an engineered loop extension in the Fc, optionally wherein the engineered loop extension places the antigens or epitopes thereof in a more favorable orientation for divalent binding of at least one B-cell receptor and/or antibody.

25. The divalent immunogen of any one of claims 9-24, wherein all or part of the antibody is humanized.

26. The divalent immunogen of any one of claims 20-25, wherein the Fc comprises amino acid substitutions corresponding to L234A/L235A/P329G in SEQ ID NO 1.

27. The divalent immunogen of any one of claims 1-26, wherein the antigen is HIV gp120, or a fragment or variant.

28. The divalent immunogen of any one of claims 1-26, wherein the epitope is the CD4 binding site of HIV gp120.

29. The divalent immunogen of claim 27, wherein the sequence of the HIV gp120 antigen comprises a sequence at least 80% identical to SEQ ID NO: 13.

30. The divalent immunogen of any one of claim 27 or claim 29, wherein the first engineered antigen mutation of the HIV gp120 antigen, or fragment or variant thereof, corresponds to 1423C of HIV gp120.

31. The divalent immunogen of any one of claims 27-30, wherein at least one of the two antigens or epitopes thereof does not comprise the N276 glycan.

32. The divalent immunogen of any one of claims 9-31, wherein the Fab domains are derived from a co-receptor binding site antibody.

33. The divalent immunogen of claim 32, wherein the co-receptor binding site antibody is 48d.

34. The divalent immunogen of claim 32 or claim 33, wherein the sequence of the Fab heavy chain comprises a sequence at least 80% identical to SEQ ID NO: 1.

35. The divalent immunogen of any one of claims 32-34, wherein the sequence of the Fab light chain comprises a sequence at least 80% identical to SEQ ID NO: 3.

36. The divalent immunogen of any one of claims 12-35, wherein the first engineered Fab mutation corresponds to D56 of the 48d heavy chain.

37. The divalent immunogen of any one of claims 13-36, wherein the second engineered Fab mutation corresponds to K126C of the 48d light chain.

38. The divalent immunogen of any one of claims 14-37, wherein the second engineered Fab mutation comprises addition of at least one amino acid between the residues corresponding to amino acids 119 to 132 of the 48d light chain, optionally wherein the second engineered mutation additionally comprises substitution of at least one of the residues corresponding to amino acids 119 to 132 of the 48d light chain.

39. The divalent immunogen of claim 38, wherein the sequence of the Fab light chain comprises any one of SEQ ID NOs: 7, 9, and 11.

40. The divalent immunogen of any one of claims 1-39, wherein the divalent immunogen provides precise positioning of two antigens or epitopes thereof such that certain B-cell receptors are preferably bound to both antigens simultaneously.

41. The divalent immunogen of claim 40, wherein the two Fab domains of the bound B-cell receptor assume an angle of about 20 degrees to about 150 degrees.

42. The divalent immunogen of claim 41, wherein the two Fab domains of the bound B-cell receptor assume an angle of about 90 degrees to about 140 degrees.

43. The divalent immunogen of claim 42, wherein the two Fab domains of the bound B-cell receptor assume an angle of about 120 degrees.

44. The divalent immunogen of any one of claims 1-43, wherein the divalent immunogen provides an antigenic spacing of about 3 to about 20 nm.

45. The divalent immunogen of claim 44, wherein the divalent immunogen provides an antigenic spacing of about 7 to about 19 nm.

46. The divalent immunogen of claim 45, wherein the divalent immunogen provides an antigenic spacing of about 13 to about 16 nm.

47. The divalent immunogen of any of one of claims 13-46, wherein the second engineered Fab mutation fixes the distance between the antigens or epitopes thereof.

48. The divalent immunogen of any one of claims 13-47, wherein the second engineered Fab mutation modifies the interaction between the antigens or epitopes thereof and certain B-cell receptors.

49. The divalent immunogen of any one of claims 13-48, wherein the second engineered Fab mutation enhances the binding avidity of certain B-cell receptors and/or antibodies for the antigen or epitope thereof by at least 1.5-fold.

50. The divalent immunogen of claim 49, wherein the second engineered Fab mutation enhances the binding avidity of certain B-cell receptors and/or antibodies for the antigen or epitope thereof by at least 10-fold.

51. The divalent immunogen of claim 50, wherein the second engineered Fab mutation enhances the binding avidity of certain B-cell receptors and/or antibodies for the antigen or epitope thereof by at least 100-fold.

52. The divalent immunogen of any one of claims 1-51, wherein the binding avidity of certain B-cell receptors is at least 1.5-fold higher for the two antigens covalently linked to the scaffold compared to the same antigen unlinked to the scaffold.

53. The divalent immunogen of claim 52, wherein the binding avidity of certain B-cell receptors is at least 10-fold higher for the two antigens covalently linked to the scaffold compared to the same antigen unlinked to the scaffold.

54. The divalent immunogen of claim 53, wherein the binding avidity of certain B-cell receptors is at least 100-fold higher for the two antigens covalently linked to the scaffold compared to the same antigen unlinked to the scaffold.

55. A multimer of the divalent immunogen of any one of claims 1-54.

56. The multimer of claim 55, wherein the monomers of the divalent immunogen are conjugated onto a nanoparticle.

57. The multimer of claim 55 or claim 56, wherein the multimer is a dimer, a trimer, or a tetramer.

58. A pharmaceutical composition comprising the divalent immunogen of any one of claims 1-54 and/or the multimer of any one of claims 55-57.

59. The pharmaceutical composition of claim 58, additionally comprising a pharmaceutically-acceptable solvent.

60. The pharmaceutical composition of claim 59, additionally comprising an adjuvant, optionally wherein the adjuvant is poly I:C.

61. Polynucleotides encoding the scaffold and two antigens or epitopes thereof of any one of claims 1-54.

62. A polynucleotide encoding a scaffold or portion thereof, wherein the encoded scaffold provides precise positioning of two antigens or epitopes thereof such that only certain B-cell receptors are preferably bound to both antigens simultaneously.

63. A polynucleotide encoding the Fab heavy chain of claim 34 or 36.

64. A polynucleotide encoding the Fab light chain of claim 35 or any one of claims 37-39.

65. A cell comprising at least one polynucleotide of any one of claims 60-64.

66. A method of identifying antibodies with preferred characteristics for divalent immunogens, comprising:
(a) providing a divalent immunogen comprising a known broadly neutralizing antibody and an antigen or epitope thereof;
(b) administering the divalent immunogen to a subject, optionally a mouse;
(c) Isolating B cells from the subject;
(d) screening the isolated B cells for ability to bind to the divalent immunogen in high avidity, bidentate fashion; and
(e) sequencing the complementarity determining regions of B cells that bind as in (d).

67. A method of generating an immune response in a patient, comprising administering the divalent immunogen of any one of claims 1-54, the multimer of any one of claims 55-57, or the pharmaceutical composition of any one of claims 58-60.

68. The method of claim 67, wherein the immune response results in an increased number of B cells expressing a B-cell receptor that binds the divalent immunogen divalently with increased avidity.

69. The method of claim 68, wherein the patient has previously been administered at least one dose of a vaccine, optionally wherein the dose was of a different vaccine.

70. The method of any one of claims 67-69, wherein the immune response results in an increased number of B cells expressing a VRC01-class B-cell receptor.

71. A method of forming a ring structure comprising the divalent immunogen of any one of class 1-54 and a B-cell receptor and/or antibody;
wherein the divalent immunogen comprises a specific antigenic spacing that allows the B-cell receptor and/or antibody to bind with a Fab-Fab angle of about 20 degrees to about 150 degrees.

72. The method of claim 71, wherein the Fab-Fab angle is about 90 degrees to about 140 degrees.

73. The method of claim 72 wherein the Fab-Fab angle is about 120 degrees.

74. The method of any one of claims 71-73, wherein the ring structure is formed in a subject.

75. The method of claim 74, wherein the subject is a human.


SEQUENCE LISTING (72 sequences)

SEQ ID NO: 1 - protein, 467 aa, Homo sapiens
MGWSCIILFLVATATGVHSEVQLVQSGAEVKKPGATVKISCKASGYTFSDFYMYWVRQAPGKGLEWMGLIDPEDACTMYAEKFRGRVTITADTSTDTGYLELSSLRSEDTAVYYCAADPWELNAFNVWGQGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 2 - DNA, 1404 bp, Homo sapiens
  source 1..1404: mol_type = genomic DNA
atgggatggtcatgtatcatcctttttctagtagcaactgcaaccggtgtacattccgaagttcaacttgtgcagtcaggagctgaggtcaagaaacccggtgctacagtgaagattagctgcaaagcaagcggctataccttttcagacttttatatgtactgggttcgccaagcccccggcaaaggtctggaatggatgggcctgattgatccagaagacgcctgcactatgtacgcagagaaattccgaggtagggttactatcactgctgacacgtccaccgatacaggctatctagagcttagctctctaagaagtgaagacacggcagtgtattattgtgctgccgacccatgggaactcaatgcctttaacgtttggggacaagggacacttgtcagcgtttcaagcgcctcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctaacgctgctggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcggcgcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggttga

SEQ ID NO: 3 - protein, 232 aa, Homo sapiens
MGWSCIILFLVATATGVHSDIQMTQSPSSVSASVGDRVTITCRASQDISTWLAWYQQKPGKAPKLLIYAASTLQSGVPSRFSGSGSGTDFSLTINSLQPEDFATYYCQQANSFFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - DNA, 699 bp, Homo sapiens
  source 1..699: mol_type = genomic DNA
atgggatggtcatgtatcatcctttttctagtagcaactgcaaccggtgtacattcagatattcaaatgacgcaaagtcctagcagtgtctctgccagtgtgggtgaccgagtcacaatcacctgtagagcttcacaggatattagtacctggttggcttggtatcaacaaaagcctggaaaggccccaaaactcctgatttacgccgcctcaacattgcaatcaggagtgccttctagatttagtgggagcgggtctgggaccgacttctcacttacaattaacagcctgcagccggaagattttgccacctattattgccaacaagctaactctttcttcacgttcggggggggaactaaagttgagattaaacgtacggtggctgcaccatctgtcttcatcttcccgccatctgatgagcagttgaaatctggaactgcctctgttgtgtgcctgctgaataacttctaccccagagaagccaaagtgcagtggaaggtggacaacgccctgcagagcggaaacagccaggaaagcgtgacagagcaggattccaaggattccacatacagcctgagcagcacactgacactgtccaaggccgactacgagaagcacaaggtgtacgcctgcgaagtgacacaccagggactgtcctcccctgtgacaaagagcttcaacagaggagaatgctga

SEQ ID NO: 5 - protein, 232 aa, synthetic construct
MGWSCIILFLVATATGVHSDIQMTQSPSSVSASVGDRVTITCRASQDISTWLAWYQQKPGKAPKLLIYAASTLQSGVPSRFSGSGSGTDFSLTINSLQPEDFATYYCQQANSFFTFGGGTKVEIKRTVAAPSVFIFPPSDEQLCSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - DNA, 699 bp, synthetic construct
  source 1..699: mol_type = other DNA
atgggatggtcatgtatcatcctttttctagtagcaactgcaaccggtgtacattcagatattcaaatgacgcaaagtcctagcagtgtctctgccagtgtgggtgaccgagtcacaatcacctgtagagcttcacaggatattagtacctggttggcttggtatcaacaaaagcctggaaaggccccaaaactcctgatttacgccgcctcaacattgcaatcaggagtgccttctagatttagtgggagcgggtctgggaccgacttctcacttacaattaacagcctgcagccggaagattttgccacctattattgccaacaagctaactctttcttcacgttcggggggggaactaaagttgagattaaacgtacggtggctgcaccatctgtcttcatcttcccgccatctgatgagcagttgtgctctggaactgcctctgttgtgtgcctgctgaataacttctaccccagagaagccaaagtgcagtggaaggtggacaacgccctgcagagcggaaacagccaggaaagcgtgacagagcaggattccaaggattccacatacagcctgagcagcacactgacactgtccaaggccgactacgagaagcacaaggtgtacgcctgcgaagtgacacaccagggactgtcctcccctgtgacaaagagcttcaacagaggagaatgctga

SEQ ID NO: 7 - protein, 236 aa, synthetic construct
MGWSCIILFLVATATGVHSDIQMTQSPSSVSASVGDRVTITCRASQDISTWLAWYQQKPGKAPKLLIYAASTLQSGVPSRFSGSGSGTDFSLTINSLQPEDFATYYCQQANSFFTFGGGTKVEIKRTVAAPSVFIFPPNDEQLLPCAGPSNASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - DNA, 711 bp, synthetic construct
  source 1..711: mol_type = other DNA
atgggatggtcatgtatcatcctttttctagtagcaactgcaaccggtgtacattcagatattcaaatgacgcaaagtcctagcagtgtctctgccagtgtgggtgaccgagtcacaatcacctgtagagcttcacaggatattagtacctggttggcttggtatcaacaaaagcctggaaaggccccaaaactcctgatttacgccgcctcaacattgcaatcaggagtgccttctagatttagtgggagcgggtctgggaccgacttctcacttacaattaacagcctgcagccggaagattttgccacctattattgccaacaagctaactctttcttcacgttcggggggggaactaaagttgagattaaacgtacggtggctgcaccatctgtcttcatcttcccgccaaacgatgaacagctgctgccttgcgcagggccgagcaacgcctctgttgtgtgcctgctgaataacttctaccccagagaagccaaagtgcagtggaaggtggacaacgccctgcagagcggaaacagccaggaaagcgtgacagagcaggattccaaggattccacatacagcctgagcagcacactgacactgtccaaggccgactacgagaagcacaaggtgtacgcctgcgaagtgacacaccagggactgtcctcccctgtgacaaagagcttcaacagaggagaatgctga

SEQ ID NO: 9 - protein, 233 aa, synthetic construct
MGWSCIILFLVATATGVHSDIQMTQSPSSVSASVGDRVTITCRASQDISTWLAWYQQKPGKAPKLLIYAASTLQSGVPSRFSGSGSGTDFSLTINSLQPEDFATYYCQQANSFFTFGGGTKVEIKRTVAAPSVFIFPPAPPEGCGSPTRTVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - DNA, 702 bp, synthetic construct
  source 1..702: mol_type = other DNA
atgggatggtcatgtatcatcctttttctagtagcaactgcaaccggtgtacattcagatattcaaatgacgcaaagtcctagcagtgtctctgccagtgtgggtgaccgagtcacaatcacctgtagagcttcacaggatattagtacctggttggcttggtatcaacaaaagcctggaaaggccccaaaactcctgatttacgccgcctcaacattgcaatcaggagtgccttctagatttagtgggagcgggtctgggaccgacttctcacttacaattaacagcctgcagccggaagattttgccacctattattgccaacaagctaactctttcttcacgttcggggggggaactaaagttgagattaaacgtacggtggctgcaccatctgtcttcatcttcccgccagcacctcctgaggggtgcgggagccctactagaactgttgtgtgcctgctgaataacttctaccccagagaagccaaagtgcagtggaaggtggacaacgccctgcagagcggaaacagccaggaaagcgtgacagagcaggattccaaggattccacatacagcctgagcagcacactgacactgtccaaggccgactacgagaagcacaaggtgtacgcctgcgaagtgacacaccagggactgtcctcccctgtgacaaagagcttcaacagaggagaatgctga

SEQ ID NO: 11 - protein, 231 aa, synthetic construct
MGWSCIILFLVATATGVHSDIQMTQSPSSVSASVGDRVTITCRASQDISTWLAWYQQKPGKAPKLLIYAASTLQSGVPSRFSGSGSGTDFSLTINSLQPEDFATYYCQQANSFFTFGGGTKVEIKRTVAAPSVFIFRAEEGCGGNTVYLVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - DNA, 696 bp, synthetic construct
  source 1..696: mol_type = other DNA
atgggatggtcatgtatcatcctttttctagtagcaactgcaaccggtgtacattcagatattcaaatgacgcaaagtcctagcagtgtctctgccagtgtgggtgaccgagtcacaatcacctgtagagcttcacaggatattagtacctggttggcttggtatcaacaaaagcctggaaaggccccaaaactcctgatttacgccgcctcaacattgcaatcaggagtgccttctagatttagtgggagcgggtctgggaccgacttctcacttacaattaacagcctgcagccggaagattttgccacctattattgccaacaagctaactctttcttcacgttcggggggggaactaaagttgagattaaacgtacggtggctgcaccatctgtcttcatcttcagagcagaggaggggtgcggggggaacactgtgtacctggtgtgcctgctgaataacttctaccccagagaagccaaagtgcagtggaaggtggacaacgccctgcagagcggaaacagccaggaaagcgtgacagagcaggattccaaggattccacatacagcctgagcagcacactgacactgtccaaggccgactacgagaagcacaaggtgtacgcctgcgaagtgacacaccagggactgtcctcccctgtgacaaagagcttcaacagaggagaatgctga

SEQ ID NO: 13 - protein, 378 aa, Human immunodeficiency virus
MPMGSLQPLATLYLLGMLVASVLAVWKDAETTLFCASDAKAYETEKHNVWATHACVPTDPNPQEIHLEGVTEEFNMWKNNMVEQMHTDIISLWDQSLKPCVKLTGGSAITQACPKVSFEPIPIHYCAPAGFAILKCKDKKFNGTGPCPSVSTVQCTHGIKPVVSTQLLLNGSLAEEEVMIRSENITNNAKNILVQFNTPVQINCTRPGNGGDIRQAHCNVSKATWNETLGKVVKQLRKHFGNNTIIRFANSSGGDLEVTTHSFNCGGEFFYCNTSGLFNSTWISNTSVQGSNSTGSNDSITLPCRIKQIINMWQRIGQAMYAPPIQGVIRCVSNITGLILTRDGCSTNSTTETFRPGGGDMRDNWRSELYKYKVVKIE

SEQ ID NO: 14 - DNA, 1137 bp, synthetic construct
  source 1..1137: mol_type = other DNA
atgcccatgggctccctgcagccgttagcaactctgtatctgctggggatgctggtagccagcgtgctggcagtgtggaaagatgctgaaaccacattgttttgtgcgagcgatgccaaagcatacgagacggaaaagcacaatgtctgggccactcatgcatgcgtgcccacagacccaaacccccaggagatccacctggaaggagtgaccgaggaattcaacatgtggaaaaacaatatggtggagcagatgcatacagacatcattagcctgtgggatcagtccctgaagccctgcgtcaaactgactggcggctccgccattacccaggcttgtcctaaagtgtcttttgagcctatcccaattcattattgcgccccagctggcttcgccatcctgaagtgtaaagataagaagttcaacggaactggcccctgcccttccgtgtctacagtccagtgtactcacgggattaagcctgtggtctctacacagctgctgctgaatggaagtctggctgaggaagaagtgatgatccggagcgagaacattaccaacaatgccaagaatatcctggtccagttcaacacaccagtgcagattaattgcacaagacccggaaatggaggagatatcagacaggctcactgtaatgtgagtaaggcaacctggaacgagacactgggcaaggtggtcaaacagctgaggaaacatttcgggaataacaccatcattcgctttgccaatagctccggaggggacctggaggtcactacccactccttcaactgcggaggcgaattcttttactgtaacacatctggcctgtttaatagtacatggatctctaacactagtgtgcagggcagtaattcaactgggtcaaacgatagcatcaccctgccatgccgaattaagcagatcattaatatgtggcagcggataggtcaggcaatgtatgctcccccaatccagggagttattcggtgcgtctctaatatcacgggacttatcttgactcgcgacggctgcagcacgaacagcacaacagaaacattccggcctggggggggagacatgcgggataactggcggagtgagctgtacaagtataaagttgttaagatcgaatga

SEQ ID NO: 15 - protein, 378 aa, unidentified
MPMGSLQPLATLYLLGMLVASVLAVWKDAETTLFCASDAKAYETEKHNVWATHACVPTDPNPQEIHLEGVTEEFNMWKNNMVEQMHTDIISLWDQSLKPCVKLTGGSAITQACPKVSFEPIPIHYCAPAGFAILKCKDKKFNGTGPCPSVSTVQCTHGIKPVVSTQLLLNGSLAEEEVMIRSENITNNAKNILVQFNTPVQINCTRPGNGGDIRQAHCNVSKATWNETLGKVVKQLRKHFGNNTIIRFANSSGGDLEVTTHSFNCGGEFFYCNTSGLFNSTWISNTSVQGSNSTGSNDSITLPCRIKQCINMWQRIGQAMYAPPIQGVIRCVSNITGLILTRDGGSTNSTTETFRPGGGDMRDNWRSELYKYKVVKIE

SEQ ID NO: 16 - DNA, 1137 bp, synthetic construct
  source 1..1137: mol_type = other DNA
atgcccatgggctccctgcagccgttagcaactctgtatctgctggggatgctggtagccagcgtgctggcagtgtggaaagatgctgaaaccacattgttttgtgcgagcgatgccaaagcatacgagacggaaaagcacaatgtctgggccactcatgcatgcgtgcccacagacccaaacccccaggagatccacctggaaggagtgaccgaggaattcaacatgtggaaaaacaatatggtggagcagatgcatacagacatcattagcctgtgggatcagtccctgaagccctgcgtcaaactgactggcggctccgccattacccaggcttgtcctaaagtgtcttttgagcctatcccaattcattattgcgccccagctggcttcgccatcctgaagtgtaaagataagaagttcaacggaactggcccctgcccttccgtgtctacagtccagtgtactcacgggattaagcctgtggtctctacacagctgctgctgaatggaagtctggctgaggaagaagtgatgatccggagcgagaacattaccaacaatgccaagaatatcctggtccagttcaacacaccagtgcagattaattgcacaagacccggaaatggaggagatatcagacaggctcactgtaatgtgagtaaggcaacctggaacgagacactgggcaaggtggtcaaacagctgaggaaacatttcgggaataacaccatcattcgctttgccaatagctccggaggggacctggaggtcactacccactccttcaactgcggaggcgaattcttttactgtaacacatctggcctgtttaatagtacatggatctctaacactagtgtgcagggcagtaattcaactgggtcaaacgatagcatcaccctgccatgccgaattaagcagtgcattaatatgtggcagcggataggtcaggcaatgtatgctcccccaatccagggagttattcggtgcgtctctaatatcacgggacttatcttgactcgcgacggcggcagcacgaacagcacaacagaaacattccggcctggggggggagacatgcgggataactggcggagtgagctgtacaagtataaagttgttaagatcgaatga

SEQ ID NO: 17 - protein, 840 aa, synthetic construct
MPMGSLQPLATLYLLGMLVASVLADIRQAHCNVSKATWNETLGKVVKQLRKHFGNNTIIRFANSSGGDLEVTTHSFNCGGEFFYCNTSGLFNSTWISNTSVQGSNSTGSNDSITLPCRIKQCINMWQRIGQAMYAPPIQGVIRCVSNITGLILTRDGGSTNSTTETFRPGGGDMRDNWRSELYKYKVVKIEGGVWKDAETTLFCASDAKAYETEKHNVWATHACVPTDPNPQEIHLEGVTEEFNMWKNNMVEQMHTDIISLWDQSLKPCVKLTGGSAITQACPKVSFEPIPIHYCAPAGFAILKCKDKKFNGTGPCPSVSTVQCTHGIKPVVSTQLLLNGSLAEEEVMIRSENITNNAKNILVQFNTPVQINCTRPGNGGGGGSGGGSGGGSEVQLVQSGAEVKKPGATVKISCKASGYTFSDFYMYWVRQAPGKGLEWMGLIDPEDACTMYAEKFRGRVTITADTSTDTGYLELSSLRSEDTAVYYCAADPWELNAFNVWGQGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 18 - DNA, 2523 bp, synthetic construct
  source 1..2523: mol_type = other DNA
atgcccatgggtagcctgcaaccactggccaccctgtatctgttagggatgttggtggcctccgtgcttgcagatatccgacaggcccattgcaatgtatctaaagccacctggaatgagacactgggtaaagtcgttaagcagttgcggaaacactttggcaataatacgatcatccggttcgctaactcctccgggggagacttggaggtcacgacccattctttcaattgcggcggagaatttttttattgcaacacctcaggcctgttcaattctacctggatttcaaatactagtgtgcagggatctaacagtaccggttcaaacgacagcataacattgccctgtcggattaaacagtgtatcaacatgtggcagcgcataggtcaggccatgtatgcccctcctattcaaggagtgatacgatgcgtgtccaacataacaggacttattctcacaagagatggaggatccaccaactctacgactgagacctttagaccaggaggtggagacatgagagataactggaggagtgagctttacaaatacaaggtcgtaaaaatcgaggggggggtgtggaaagatgccgagactacgttattctgcgcctcagacgccaaggcctatgaaacagagaaacacaacgtgtgggcaacccatgcatgtgttccgaccgaccccaatccacaggagatccacctggaaggcgtcacagaagaattcaacatgtggaagaacaatatggtggagcagatgcacaccgatattattagtctgtgggaccaatcactgaaaccatgtgtcaaacttaccggcggtagcgccatcacccaggcatgtcctaaagtaagtttcgagccaatccccatccattattgcgcacctgccggcttcgcaatcctgaaatgtaaggataagaagttcaatggcacaggcccttgtccgagcgtgagcaccgtccaatgcacacacggaattaaacctgtagtcagcactcagctgctgctgaatggctccctggcagaagaggaagtcatgatccgctcagaaaacatcacaaataacgctaaaaacattctggttcaatttaacacgcctgtccagatcaactgtaccaggccgggcaacggaggtggtggtggcagtggggggggttcaggtggcggctccgaagttcaacttgtgcagtcaggagctgaggtcaagaaacccggtgctacagtgaagattagctgcaaagcaagcggctataccttttcagacttttatatgtactgggttcgccaagcccccggcaaaggtctggaatggatgggcctgattgatccagaagacgcctgcactatgtacgcagagaaattccgaggtagggttactatcactgctgacacgtccaccgatacaggctatctagagcttagctctctaagaagtgaagacacggcagtgtattattgtgctgccgacccatgggaactcaatgcctttaacgtttggggacaagggacacttgtcagcgtttcaagcgcctcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctaacgctgctggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcggcgcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggttga

SEQ ID NO: 19 - protein, 270 aa, synthetic construct
MGWSCIILFLVATATGVHSEVQLVQSGAEVKKPGATVKISCKASGYTFSDFYMYWVRQAPGKGLEWMGLIDPEDACTMYAEKFRGRVTITADTSTDTGYLELSSLRSEDTAVYYCAADPWELNAFNVWGQGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVTANTEVTPESVRAALRELFGDEELLAWAAELLRA

SEQ ID NO: 20 - DNA, 813 bp, synthetic construct
  source 1..813: mol_type = other DNA
atgggatggtcatgtatcatcctttttctagtagcaactgcaaccggtgtacattccgaagttcaacttgtgcagtcaggagctgaggtcaagaaacccggtgctacagtgaagattagctgcaaagcaagcggctataccttttcagacttttatatgtactgggttcgccaagcccccggcaaaggtctggaatggatgggcctgattgatccagaagacgcctgcactatgtacgcagagaaattccgaggtagggttactatcactgctgacacgtccaccgatacaggctatctagagcttagctctctaagaagtgaagacacggcagtgtattattgtgctgccgacccatgggaactcaatgcctttaacgtttggggacaagggacacttgtcagcgtttcaagcgcctcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttacagctaatacagaagtgacccctgagagcgtgcgggccgcactgcgcgagctgttcggagatgaggaactgctggcctgggccgcagagcttctgagagcctga

SEQ ID NO: 21 - protein, 277 aa, synthetic construct
MGWSCIILFLVATATGVHSDIQMTQSPSSVSASVGDRVTITCRASQDISTWLAWYQQKPGKAPKLLIYAASTLQSGVPSRFSGSGSGTDFSLTINSLQPEDFATYYCQQANSFFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTVSYEKLGPGLWIYQTIQDGKLLSISTNAKPSTPLEEKVRAVAIREKLIAENAA

SEQ ID NO: 22 - DNA, 834 bp, synthetic construct
  source 1..834: mol_type = other DNA
atgggatggtcatgtatcatcctttttctagtagcaactgcaaccggtgtacattcagatattcaaatgacgcaaagtcctagcagtgtctctgccagtgtgggtgaccgagtcacaatcacctgtagagcttcacaggatattagtacctggttggcttggtatcaacaaaagcctggaaaggccccaaaactcctgatttacgccgcctcaacattgcaatcaggagtgccttctagatttagtgggagcgggtctgggaccgacttctcacttacaattaacagcctgcagccggaagattttgccacctattattgccaacaagctaactctttcttcacgttcggggggggaactaaagttgagattaaacgtacggtggctgcaccatctgtcttcatcttcccgccatctgatgagcagttgaaatctggaactgcctctgttgtgtgcctgctgaataacttctaccccagagaagccaaagtgcagtggaaggtggacaacgccctgcagagcggaaacagccaggaaagcgtgacagagcaggattccaaggattccacatacagcctgagcagcacactgacactgtccaaggccgactacgagaagcacaaggtgtacgcctgcgaagtgacacaccagggactgtcctcccctgtgacagtgtcctacgagaagcttggacccggtctgtggatctaccagactattcaggatggcaagctcctgtcaatttccactaatgcaaagcccagcactccactggaagagaaagtgcgagctgtcgccatcagggagaagcttatcgctgagaacgccgcctga

SEQ ID NO: 23 - protein, 648 aa, synthetic construct
MPMGSLQPLATLYLLGMLVASVLASAITQACPKVSFEPIPIHYCAPAGFAILKCKDKKFNGTGPCPSVSTVQCTHGIKPVVSTQLLLNGSLAEEEVMIRSENITNNAKNILVQFNTPVQINCTRPGNGGDIRQAHCNVSKATWNETLGKVVKQLRKHFGNNTIIRFANSSGGDLEVTTHSFNCGGEFFYCNTSGLFNSTWISNTSVQGSNSTGSNDSITLPCRIKQCINMWQRIGQAMYAPPIQGVIRCVSNITGLILTRDGGSTNSTTETFRPGGGDMRDNWRSELYKYKVVKIEGGVWKDAETTLFCASDAKAYETEKHNVWATHACVPTDPNPQEIHLEGVTEEFNMWKNNMVEQMHTDIISLWDQSLKPCVKLTGGGSGGGSGGGSDIQMTQSPSSVSASVGDRVTITCRASQDISTWLAWYQQKPGKAPKLLIYAASTLQSGVPSRFSGSGSGTDFSLTINSLQPEDFATYYCQQANSFFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTVSYEKLGPGLWIYQTIQDGKLLSISTNAKPSTPLEEKVRAVAIREKLIAENAA

SEQ ID NO: 24 - DNA, 1947 bp, synthetic construct
  source 1..1947: mol_type = other DNA
atgcccatgggcagcttacagccccttgccacactgtatctcttggggatgcttgttgcctccgtactcgccagcgcaatcactcaggcttgtccaaaggtttcctttgaaccgatacccattcactattgtgctcccgccggcttcgccatccttaagtgtaaggacaagaaattcaatggcactggcccgtgtccctccgtgtccaccgtgcagtgcacccatggcataaaacccgtggtgagcactcagctgctgctgaacggttcattggccgaggaagaagttatgatcagatctgagaacatcacgaataacgctaaaaatatcctggttcagttcaacaccccagtacaaatcaattgtactagaccaggcaacggcggcgacatccgccaggctcattgtaacgtctccaaggcaacttggaatgaaacactcgggaaagtggtgaaacagctgcgcaagcattttgggaataacaccatcatacgattcgccaatagcagcgggggagacctggaggttaccactcacagtttcaactgtggaggcgaatttttctattgtaacacatcaggactgttcaattctacatggataagtaacacatcagtccaggggtcaaacagtaccggaagtaatgacagcatcaccctgccctgtagaattaagcagtgcattaacatgtggcagcggatcggccaggctatgtacgcccctcccatacagggcgttatccgttgcgtgagcaatattacaggcttgattttaactagagacggcggttccacaaattctacaaccgaaaccttcagacccggcgggggcgacatgcgtgacaactggcgctcagaactctacaaatacaaagttgtgaaaattgagggaggcgtatggaaggacgctgaaaccaccctgttctgcgcaagcgacgccaaggcatatgaaactgagaaacacaacgtatgggccacacacgcttgtgtccccacagatcccaacccccaagagattcacctcgagggtgttacagaggaattcaacatgtggaaaaacaacatggtcgagcagatgcacacagacatcatatccctctgggatcaaagtctgaagccctgcgtgaaactgactggcggcggtagtgggggtggatcaggcggtggtagtgatattcaaatgacgcaaagtcctagcagtgtctctgccagtgtgggtgaccgagtcacaatcacctgtagagcttcacaggatattagtacctggttggcttggtatcaacaaaagcctggaaaggccccaaaactcctgatttacgccgcctcaacattgcaatcaggagtgccttctagatttagtgggagcgggtctgggaccgacttctcacttacaattaacagcctgcagccggaagattttgccacctattattgccaacaagctaactctttcttcacgttcggggggggaactaaagttgagattaaacgtacggtggctgcaccatctgtcttcatcttcccgccatctgatgagcagttgaaatctggaactgcctctgttgtgtgcctgctgaataacttctaccccagagaagccaaagtgcagtggaaggtggacaacgccctgcagagcggaaacagccaggaaagcgtgacagagcaggattccaaggattccacatacagcctgagcagcacactgacactgtccaaggccgactacgagaagcacaaggtgtacgcctgcgaagtgacacaccagggactgtcctcccctgtgacagtgtcctacgagaagcttggacccggtctgtggatctaccagactattcaggatggcaagctcctgtcaatttccactaatgcaaagcccagcactccactggaagagaaagtgcgagctgtcgccatcagggagaagcttatcgctgagaacgccgcctga

SEQ ID NO: 25 - protein, 279 aa, synthetic construct
MGWSCIILFLVATATGVHSEVQLVQSGAEVKKPGATVKISCKASGYTFSDFYMYWVRQAPGKGLEWMGLIDPEDACTMYAEKFRGRVTITADTSTDTGYLELSSLRSEDTAVYYCAADPWELNAFNVWGQGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVSTPVEAVLPPEALAHLRAFLALLPLEAALAVAHAVRQALIAVQ

SEQ ID NO: 26 - DNA, 837 bp, synthetic construct
  source 1..837: mol_type = other DNA
atgggatggtcatgtatcatcctttttctagtagcaactgcaaccggtgtacattccgaagttcaacttgtgcagtcaggagctgaggtcaagaaacccggtgctacagtgaagattagctgcaaagcaagcggctataccttttcagacttttatatgtactgggttcgccaagcccccggcaaaggtctggaatggatgggcctgattgatccagaagacgcctgcactatgtacgcagagaaattccgaggtagggttactatcactgctgacacgtccaccgatacaggctatctagagcttagctctctaagaagtgaagacacggcagtgtattattgtgctgccgacccatgggaactcaatgcctttaacgtttggggacaagggacacttgtcagcgtttcaagcgcctcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtttctactccagtcgaggcagtgcttcctcccgaggcacttgctcatttgagagctttcctggctctgcttccgctggaggccgctcttgccgtggctcacgccgtgcggcaggccctgatcgcagtccaa

SEQ ID NO: 27 - protein, 267 aa, synthetic construct
MGWSCIILFLVATATGVHSDIQMTQSPSSVSASVGDRVTITCRASQDISTWLAWYQQKPGKAPKLLIYAASTLQSGVPSRFSGSGSGTDFSLTINSLQPEDFATYYCQQANSFFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTARILRVNDVVFTSTQTETSGEWTVNSTPLDQLLLDIYLEAQAA

SEQ ID NO: 28 - DNA, 801 bp, synthetic construct
  source 1..801: mol_type = other DNA
atgggatggtcatgtatcatcctttttctagtagcaactgcaaccggtgtacattcagatattcaaatgacgcaaagtcctagcagtgtctctgccagtgtgggtgaccgagtcacaatcacctgtagagcttcacaggatattagtacctggttggcttggtatcaacaaaagcctggaaaggccccaaaactcctgatttacgccgcctcaacattgcaatcaggagtgccttctagatttagtgggagcgggtctgggaccgacttctcacttacaattaacagcctgcagccggaagattttgccacctattattgccaacaagctaactctttcttcacgttcggggggggaactaaagttgagattaaacgtacggtggctgcaccatctgtcttcatcttcccgccatctgatgagcagttgaaatctggaactgcctctgttgtgtgcctgctgaataacttctaccccagagaagccaaagtgcagtggaaggtggacaacgccctgcagagcggaaacagccaggaaagcgtgacagagcaggattccaaggattccacatacagcctgagcagcacactgacactgtccaaggccgactacgagaagcacaaggtgtacgcctgcgaagtgacacaccagggactgtcctcccctgtgacagccagaatcctgagggtgaacgatgtagtatttacttctacccagaccgaaacatctggagagtggactgttaactctacacctctggatcagctcctgcttgatatctacttggaggcccaggccgcc

SEQ ID NO: 29 - protein, 284 aa, synthetic construct
MGWSCIILFLVATATGVHSEVQLVQSGAEVKKPGATVKISCKASGYTFSDFYMYWVRQAPGKGLEWMGLIDPEDACTMYAEKFRGRVTITADTSTDTGYLELSSLRSEDTAVYYCAADPWELNAFNVWGQGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKNSVTLTSTAADRELEEALRKLAPEAKFVLVGTLPDGSTLVIVFVS

SEQ ID NO: 30 - DNA, 852 bp, synthetic construct
  source 1..852: mol_type = other DNA
atgggatggtcatgtatcatcctttttctagtagcaactgcaaccggtgtacattccgaagttcaacttgtgcagtcaggagctgaggtcaagaaacccggtgctacagtgaagattagctgcaaagcaagcggctataccttttcagacttttatatgtactgggttcgccaagcccccggcaaaggtctggaatggatgggcctgattgatccagaagacgcctgcactatgtacgcagagaaattccgaggtagggttactatcactgctgacacgtccaccgatacaggctatctagagcttagctctctaagaagtgaagacacggcagtgtattattgtgctgccgacccatgggaactcaatgcctttaacgtttggggacaagggacacttgtcagcgtttcaagcgcctcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcctaaaaactctgtgacactgacatccacggctgccgacagggagctcgaggaggcattgaggaaactggcccccgaggccaagtttgtattggtcggaaccctgcccgatggctctaccctcgttattgtgtttgtttcc

SEQ ID NO: 31 - protein, 282 aa, synthetic construct
MGWSCIILFLVATATGVHSDIQMTQSPSSVSASVGDRVTITCRASQDISTWLAWYQQKPGKAPKLLIYAASTLQSGVPSRFSGSGSGTDFSLTINSLQPEDFATYYCQQANSFFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNKYPGVVVTETVDEATGTTTITLNTKNYKAVRLAIKIAEALHKLRPGSTTVAVIS

SEQ ID NO: 32 - DNA, 846 bp, synthetic construct
  source 1..846: mol_type = other DNA
atgggatggtcatgtatcatcctttttctagtagcaactgcaaccggtgtacattcagatattcaaatgacgcaaagtcctagcagtgtctctgccagtgtgggtgaccgagtcacaatcacctgtagagcttcacaggatattagtacctggttggcttggtatcaacaaaagcctggaaaggccccaaaactcctgatttacgccgcctcaacattgcaatcaggagtgccttctagatttagtgggagcgggtctgggaccgacttctcacttacaattaacagcctgcagccggaagattttgccacctattattgccaacaagctaactctttcttcacgttcggggggggaactaaagttgagattaaacgtacggtggctgcaccatctgtcttcatcttcccgccatctgatgagcagttgaaatctggaactgcctctgttgtgtgcctgctgaataacttctaccccagagaagccaaagtgcagtggaaggtggacaacgccctgcagagcggaaacagccaggaaagcgtgacagagcaggattccaaggattccacatacagcctgagcagcacactgacactgtccaaggccgactacgagaagcacaaggtgtacgcctgcgaagtgacacaccagggactgtcctcccctgtgacaaagagcttcaataaatatccaggcgtggtggttaccgaaacagtcgatgaggcaaccggcaccactacaattaccctcaacactaaaaactataaggcagttcgcctggccatcaaaattgccgaagccctgcataaactgcgtccgggctccaccaccgttgccgttatcagt

SEQ ID NO: 33 - protein, 283 aa, synthetic construct
MGWSCIILFLVATATGVHSEVQLVQSGAEVKKPGATVKISCKASGYTFSDFYMYWVRQAPGKGLEWMGLIDPEDACTMYAEKFRGRVTITADTSTDTGYLELSSLRSEDTAVYYCAADPWELNAFNVWGQGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVPTNKVVTVTIEVKEEELPEDVSVRIRMEIDISEKTGKVEMRIEVSKS

SEQ ID NO: 34 - DNA, 849 bp, synthetic construct
  source 1..849: mol_type = other DNA
atgggatggtcatgtatcatcctttttctagtagcaactgcaaccggtgtacattccgaagttcaacttgtgcagtcaggagctgaggtcaagaaacccggtgctacagtgaagattagctgcaaagcaagcggctataccttttcagacttttatatgtactgggttcgccaagcccccggcaaaggtctggaatggatgggcctgattgatccagaagacgcctgcactatgtacgcagagaaattccgaggtagggttactatcactgctgacacgtccaccgatacaggctatctagagcttagctctctaagaagtgaagacacggcagtgtattattgtgctgccgacccatgggaactcaatgcctttaacgtttggggacaagggacacttgtcagcgtttcaagcgcctcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttcccacaaacaaagtggttactgtgacaatcgaggtcaaggaggaggagcttcccgaggacgtgtctgtgaggattcgtatggagatcgatattagtgagaagacaggaaaggttgaaatgcgaattgaagttagcaagagc

SEQ ID NO: 35 - protein, 272 aa, synthetic construct
MGWSCIILFLVATATGVHSDIQMTQSPSSVSASVGDRVTITCRASQDISTWLAWYQQKPGKAPKLLIYAASTLQSGVPSRFSGSGSGTDFSLTINSLQPEDFATYYCQQANSFFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTVTIHRFKPNNVVTIEKLPSGKIKVTLTAENVEEALVNAYRLALELAEE

SEQ ID NO: 36 - DNA, 816 bp, synthetic construct
  source 1..816: mol_type = other DNA
atgggatggtcatgtatcatcctttttctagtagcaactgcaaccggtgtacattcagatattcaaatgacgcaaagtcctagcagtgtctctgccagtgtgggtgaccgagtcacaatcacctgtagagcttcacaggatattagtacctggttggcttggtatcaacaaaagcctggaaaggccccaaaactcctgatttacgccgcctcaacattgcaatcaggagtgccttctagatttagtgggagcgggtctgggaccgacttctcacttacaattaacagcctgcagccggaagattttgccacctattattgccaacaagctaactctttcttcacgttcggggggggaactaaagttgagattaaacgtacggtggctgcaccatctgtcttcatcttcccgccatctgatgagcagttgaaatctggaactgcctctgttgtgtgcctgctgaataacttctaccccagagaagccaaagtgcagtggaaggtggacaacgccctgcagagcggaaacagccaggaaagcgtgacagagcaggattccaaggattccacatacagcctgagcagcacactgacactgtccaaggccgactacgagaagcacaaggtgtacgcctgcgaagtgacacaccagggactgtcctcccctgtgacagttaccatccacaggtttaagcccaataatgtggtcaccatcgagaaactgcccagcgggaaaatcaaagtgaccctgacagctgaaaacgttgaagaagcattggttaacgcctacaggctggctcttgaactagcagaggag

SEQ ID NO: 37 - protein, 285 aa, synthetic construct
MGWSCIILFLVATATGVHSEVQLVQSGAEVKKPGATVKISCKASGYTFSDFYMYWVRQAPGKGLEWMGLIDPEDACTMYAEKFRGRVTITADTSTDTGYLELSSLRSEDTAVYYCAADPWELNAFNVWGQGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKNWVKATVKVEDGVVEAEFDSGGSKNIIEGLANSMKLLAEVALELLS

SEQ ID NO: 38 - DNA, 855 bp, synthetic construct
  source 1..855: mol_type = other DNA
atgggatggtcatgtatcatcctttttctagtagcaactgcaaccggtgtacattccgaagttcaacttgtgcagtcaggagctgaggtcaagaaacccggtgctacagtgaagattagctgcaaagcaagcggctataccttttcagacttttatatgtactgggttcgccaagcccccggcaaaggtctggaatggatgggcctgattgatccagaagacgcctgcactatgtacgcagagaaattccgaggtagggttactatcactgctgacacgtccaccgatacaggctatctagagcttagctctctaagaagtgaagacacggcagtgtattattgtgctgccgacccatgggaactcaatgcctttaacgtttggggacaagggacacttgtcagcgtttcaagcgcctcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgaacccaagaactgggtgaaggccactgttaaagtcgaggacggggtggtggaagccgagtttgactccggtgggagtaaaaacatcattgagggactggccaattctatgaaactgctcgctgaggtggctctagagctgctcagc

SEQ ID NO: 39 - protein, 266 aa, synthetic construct
MGWSCIILFLVATATGVHSDIQMTQSPSSVSASVGDRVTITCRASQDISTWLAWYQQKPGKAPKLLIYAASTLQSGVPSRFSGSGSGTDFSLTINSLQPEDFATYYCQQANSFFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRLSSGTWATIEKKEDPGEMNVTANVANVISAKIKVEFK

SEQ ID NO: 40 - DNA, 798 bp, synthetic construct
  source 1..798: mol_type = other DNA
atgggatggtcatgtatcatcctttttctagtagcaactgcaaccggtgtacattcagatattcaaatgacgcaaagtcctagcagtgtctctgccagtgtgggtgaccgagtcacaatcacctgtagagcttcacaggatattagtacctggttggcttggtatcaacaaaagcctggaaaggccccaaaactcctgatttacgccgcctcaacattgcaatcaggagtgccttctagatttagtgggagcgggtctgggaccgacttctcacttacaattaacagcctgcagccggaagattttgccacctattattgccaacaagctaactctttcttcacgttcggggggggaactaaagttgagattaaacgtacggtggctgcaccatctgtcttcatcttcccgccatctgatgagcagttgaaatctggaactgcctctgttgtgtgcctgctgaataacttctaccccagagaagccaaagtgcagtggaaggtggacaacgccctgcagagcggaaacagccaggaaagcgtgacagagcaggattccaaggattccacatacagcctgagcagcacactgacactgtccaaggccgactacgagaagcacaaggtgtacgcctgcgaagtgacacaccagggactgtcctcccctgtgacaaagtcttttaataggctaagttctggaacatgggccaccatcgagaagaaggaagatcccggcgagatgaacgtgacagccaacgtagcaaacgttattagcgctaagatcaaggtcgagttcaag

SEQ ID NO: 41 - protein, 277 aa, synthetic construct
MGWSCIILFLVATATGVHSEVQLVQSGAEVKKPGATVKISCKASGYTFSDFYMYWVRQAPGKGLEWMGLIDPEDACTMYAEKFRGRVTITADTSTDTGYLELSSLRSEDTAVYYCAADPWELNAFNVWGQGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKEVLNRVTVKVPAELTEAEIAMLKTLLKALGEAIAKAAK

SEQ ID NO: 42 - DNA, 831 bp, synthetic construct
  source 1..831: mol_type = other DNA
atgggatggtcatgtatcatcctttttctagtagcaactgcaaccggtgtacattccgaagttcaacttgtgcagtcaggagctgaggtcaagaaacccggtgctacagtgaagattagctgcaaagcaagcggctataccttttcagacttttatatgtactgggttcgccaagcccccggcaaaggtctggaatggatgggcctgattgatccagaagacgcctgcactatgtacgcagagaaattccgaggtagggttactatcactgctgacacgtccaccgatacaggctatctagagcttagctctctaagaagtgaagacacggcagtgtattattgtgctgccgacccatgggaactcaatgcctttaacgtttggggacaagggacacttgtcagcgtttcaagcgcctcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaaagaggtgctcaaccgcgtgaccgtgaaagtgcccgccgaattgactgaggccgaaattgccatgctcaagactctcctgaaagccctcggtgaggctattgctaaggccgctaaa

SEQ ID NO: 43 - protein, 282 aa, synthetic construct
MGWSCIILFLVATATGVHSDIQMTQSPSSVSASVGDRVTITCRASQDISTWLAWYQQKPGKAPKLLIYAASTLQSGVPSRFSGSGSGTDFSLTINSLQPEDFATYYCQQANSFFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNKETGLETDVDPKDADTPEYKRVTVYTPLTAEDVELFKKLAEKLAEAIYEAAKER

SEQ ID NO: 44 - DNA, 846 bp, synthetic construct
  source 1..846: mol_type = other DNA
atgggatggtcatgtatcatcctttttctagtagcaactgcaaccggtgtacattcagatattcaaatgacgcaaagtcctagcagtgtctctgccagtgtgggtgaccgagtcacaatcacctgtagagcttcacaggatattagtacctggttggcttggtatcaacaaaagcctggaaaggccccaaaactcctgatttacgccgcctcaacattgcaatcaggagtgccttctagatttagtgggagcgggtctgggaccgacttctcacttacaattaacagcctgcagccggaagattttgccacctattattgccaacaagctaactctttcttcacgttcggggggggaactaaagttgagattaaacgtacggtggctgcaccatctgtcttcatcttcccgccatctgatgagcagttgaaatctggaactgcctctgttgtgtgcctgctgaataacttctaccccagagaagccaaagtgcagtggaaggtggacaacgccctgcagagcggaaacagccaggaaagcgtgacagagcaggattccaaggattccacatacagcctgagcagcacactgacactgtccaaggccgactacgagaagcacaaggtgtacgcctgcgaagtgacacaccagggactgtcctcccctgtgacaaagtccttcaacaaggagaccggactcgagacagatgtggatcccaaagatgcagacactcctgagtacaaaagggtgaccgtctatactccgctgaccgcagaggatgtggaactgtttaagaagcttgctgagaagctggccgaagccatctatgaagccgccaaagaaagg

SEQ ID NO: 45 - protein, 278 aa, synthetic construct
MGWSCIILFLVATATGVHSEVQLVQSGAEVKKPGATVKISCKASGYTFSDFYMYWVRQAPGKGLEWMGLIDPEDACTMYAEKFRGRVTITADTSTDTGYLELSSLRSEDTAVYYCAADPWELNAFNVWGQGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKNGFSLSGSPEEVVAQALKHHTGDEKVAKMGVALVKELLA

SEQ ID NO: 46 - DNA, 834 bp, synthetic construct
  source 1..834: mol_type = other DNA
atgggatggtcatgtatcatcctttttctagtagcaactgcaaccggtgtacattccgaagttcaacttgtgcagtcaggagctgaggtcaagaaacccggtgctacagtgaagattagctgcaaagcaagcggctataccttttcagacttttatatgtactgggttcgccaagcccccggcaaaggtctggaatggatgggcctgattgatccagaagacgcctgcactatgtacgcagagaaattccgaggtagggttactatcactgctgacacgtccaccgatacaggctatctagagcttagctctctaagaagtgaagacacggcagtgtattattgtgctgccgacccatgggaactcaatgcctttaacgtttggggacaagggacacttgtcagcgtttcaagcgcctcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaagaacggcttcagtctttctggctcccctgaagaggtggtggcacaggcactgaagcatcataccggagatgaaaaggtggccaagatgggcgtcgcactagtgaaggagctgttggct

SEQ ID NO: 47 - protein, 273 aa, synthetic construct
MGWSCIILFLVATATGVHSDIQMTQSPSSVSASVGDRVTITCRASQDISTWLAWYQQKPGKAPKLLIYAASTLQSGVPSRFSGSGSGTDFSLTINSLQPEDFATYYCQQANSFFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRYPSGVIVYRTTSNGSSSYGIDANLKTLEALLEFIEAVVEIYKER

SEQ ID NO: 48 - DNA, 819 bp, synthetic construct
  source 1..819: mol_type = other DNA
atgggatggtcatgtatcatcctttttctagtagcaactgcaaccggtgtacattcagatattcaaatgacgcaaagtcctagcagtgtctctgccagtgtgggtgaccgagtcacaatcacctgtagagcttcacaggatattagtacctggttggcttggtatcaacaaaagcctggaaaggccccaaaactcctgatttacgccgcctcaacattgcaatcaggagtgccttctagatttagtgggagcgggtctgggaccgacttctcacttacaattaacagcctgcagccggaagattttgccacctattattgccaacaagctaactctttcttcacgttcggggggggaactaaagttgagattaaacgtacggtggctgcaccatctgtcttcatcttcccgccatctgatgagcagttgaaatctggaactgcctctgttgtgtgcctgctgaataacttctaccccagagaagccaaagtgcagtggaaggtggacaacgccctgcagagcggaaacagccaggaaagcgtgacagagcaggattccaaggattccacatacagcctgagcagcacactgacactgtccaaggccgactacgagaagcacaaggtgtacgcctgcgaagtgacacaccagggactgtcctcccctgtgacaaagagtttcaaccggtatcccagcggcgtgattgtatacaggacgacctccaacggctccagcagctacggcattgatgctaatctgaagacactggaagcactgctggaatttatcgaggcagtggttgagatctacaaagaaagg

SEQ ID NO: 49 - protein, 492 aa, synthetic construct
MGWSCIILFLVATATGVHSEVQLVQSGAEVKKPGATVKISCKASGYTFSDFYMYWVRQAPGKGLEWMGLIDPEDACTMYAEKFRGRVTITADTSTDTGYLELSSLRSEDTAVYYCAADPWELNAFNVWGQGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGGSGGSGGSGRGVPHIVMVDAYKRYK

SEQ ID NO: 50 - DNA, 2523 bp, synthetic construct; identical to SEQ ID NO: 18
  source 1..2523: mol_type = other DNA

SEQ ID NO: 51 - protein, 492 aa, synthetic construct; identical to SEQ ID NO: 49

SEQ ID NO: 52 - DNA, 2523 bp, synthetic construct; identical to SEQ ID NO: 18
  source 1..2523: mol_type = other DNA

SEQ ID NO: 53 - protein, 856 aa, Human immunodeficiency virus
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 54 - protein, 856 aa, Human immunodeficiency virus
MRVKETQMNWPNLWKWGTLILGLVIICSASDNLWVTVYYGVPVWRDADTTLFCASDAKAHETEVHNIWATHACVPTDPNPQEIYMENVTENFNMWRNNMVEQMQEDVISLWDQSLKPCVKLTPLCVTLNCINANLTNDKNTINVSNIIGNLTDEVKNCSFNMTTELRDKKQKVHALFYKLDLAQINNSTSEYRLINCNTSVIKQACPKISFDPIPIHYCTPAGYAILKCNDKNFNGTGPCKNVSSVQCTHGIKPVVSTQLLLNGSLAEDEIIIRSENLTNNAKTIIVHLNKSVEINCTRPSNNTRTSIRIGPGQVFYQTGDIVGDIRKAYCEINGTKWYEALKRVTEKLKEHFKNKTITFQPPSGGDLEITMHHFNCRGEFFYCNTSRLFNNTCIGNETREGCNGTIILPCKIKQIINMWQGVGQAMYAPPISGRINCVSNITGILLTRDGGANNMSNETFRPGGGNIKDNWRSELYKYKVVQIEPLGIAPTRAKRRVVEREKRAVGIGAMIFGFLGAAGSTMGAASITLTVQARQLLSGIVQQQSNLLRAIEAHEHLLQLTVWGIKQLQARVLAVERYLKDQKFLGLWGCSGKIICTTAVPWNSTWSNKSYEEIWYNMTWIEWEREISNYTYQIYEILTESQDQQDRNEKDLLELDKWASLWNWFDITDWLWYIKIFIMIVGGLIGLRIIFAVLSIVNRVRQGYSPLSFQTPIHHQREPDRPERIEEGGGEQDRDRSVRLVSGFLALAWDDLRSLCLFSYHRLRDLISIAARTVELLGHSSLKGLRRGWEALKYLGNLLLYWGQELKISVISLFDATAIVVAGWTDRVIEVAQGAWRAILHIPRRIRQGFERALL

SEQ ID NO: 55 - protein, 566 aa, Influenza A virus
MKAKLFVLFCAFNALKADTICVGYHANNSTDTVDTILKKNVTVTHSVNLLENTHNGKLCSLNGKAPLQLENCNVAGWILGNPECDFLLTENSWSYIIETSNSRNGTCYPGEFTNYEELREQLSTVSSFERFEIFPKVTSWPSHETTKGTTVACSHSGANSFYRNLLWIVKKGDSYPKLSKSYTNNKGKEVLVIWGVHHPPTYSDQQTLYQNNHTYVSVGSSKYYQRFTPEIVARPKIREQAGRMNYYWLLLDQGDTITFEATGNLIAPWHAFALDKGSNSGIMMSDAHVRNCTTKCQTPHGALKSNLPFQNVHPITIGECPKYVKSTHLRMATGLRNIPSIQSRGLFGAIAGFIEGGWTGMIDGWYGYHHQNEQGSGYAADKKSTQIAIDGISNKVNSIIEKMNIQFTSVGKEFNNLEKRIENLNKKVDDGFLDVWTYNAELLILLENERTLDFHDFNVRNLYERVKSQLRNNAKEIGNGCFEFYHKCDNECMESVKNGTYNYPRYSEESKLNREKIDGVKLESLGVHQILAIYSTVASSLILLVSLGAIVFWMCSNGSLQCRICI

SEQ ID NO: 56 - protein, 584 aa, Influenza B virus
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVKPVTSGCFPIMHDRTKIRQLPNLLRGYENIRLSTQNVIDAEKAPGGPYRLGTSGSCPNATSKSGFFATMAWAVPKDNNKNATNPLTVEVPYICTEGEDQITVWGFHSDDKTQMKNLYGDSNPQKFTSSANGVTTHYVSQIGSFPDQTEDGGLPQSGRIVVDYMMQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 57 - protein, 1273 aa, Severe acute respiratory syndrome-related coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 58 - protein, 356 aa, Hepatitis C virus
VDAETHVTGGNAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKYPEATYSRCGSGPRITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLW

SEQ ID NO: 59 - protein, 564 aa, Respiratory syncytial virus
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKQKQQAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 60 - protein, 676 aa, Ebola virus
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYASGKRSNTTGKLIWKVNPEIDTTIGEWAFWETKKNLTRKIRSEELSFTAVSNGPKNISGQSPARTSSDPETNTTNEDHKIMASENSSAMVQVHSQGRKAAVSHLTTLATISTSPQSLTTKPGPDNSTHNTPVYKLDISEATQVGQHHRRADNDSTASDTPPATTAAGPLKAENTNTSKSADSLDLATTTSPQNYSETAGNNNTHHQDTGEESASSGKLGLITNTIAGVAGLITGGRRTRREVIVNAQPKCNPNLHYWTTQDEGAAIGLAWIPYFGPAAEGIYTEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 61 - protein, 603 aa, synthetic construct
MPMGSLQPLATLYLLGMLVASVLASAITQACPKVSFEPIPIHYCAPAGFAILKCKDKKFNGTGPCPSVSTVQCTHGIKPVVSTQLLLNGSLAEEEVMIRSENITNNAKNILVQFNTPVQINCTRPGNGGDIRQAHCNVSKATWNETLGKVVKQLRKHFGNNTIIRFANSSGGDLEVTTHSFNCGGEFFYCNTSGLFNSTWISNTSVQGSNSTGSNDSITLPCRIKQCINMWQRIGQAMYAPPIQGVIRCVSNITGLILTRDGGSTNSTTETFRPGGGDMRDNWRSELYKYKVVKIEGGVWKDAETTLFCASDAKAYETEKHNVWATHACVPTDPNPQEIHLEGVTEEFNMWKNNMVEQMHTDIISLWDQSLKPCVKLTGGGSGGGSGGGSDIQMTQSPSSVSASVGDRVTITCRASQDISTWLAWYQQKPGKAPKLLIYAASTLQSGVPSRFSGSGSGTDFSLTINSLQPEDFATYYCQQANSFFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - DNA, 1812 bp, synthetic construct
  source 1..1812: mol_type = other DNA
atgcccatgggcagcttacagccccttgccacactgtatctcttggggatgcttgttgcctccgtactcgccagcgcaatcactcaggcttgtccaaaggtttcctttgaaccgatacccattcactattgtgctcccgccggcttcgccatccttaagtgtaaggacaagaaattcaatggcactggcccgtgtccctccgtgtccaccgtgcagtgcacccatggcataaaacccgtggtgagcactcagctgctgctgaacggttcattggccgaggaagaagttatgatcagatctgagaacatcacgaataacgctaaaaatatcctggttcagttcaacaccccagtacaaatcaattgtactagaccaggcaacggcggcgacatccgccaggctcattgtaacgtctccaaggcaacttggaatgaaacactcgggaaagtggtgaaacagctgcgcaagcattttgggaataacaccatcatacgattcgccaatagcagcgggggagacctggaggttaccactcacagtttcaactgtggaggcgaatttttctattgtaacacatcaggactgttcaattctacatggataagtaacacatcagtccaggggtcaaacagtaccggaagtaatgacagcatcaccctgccctgtagaattaagcagtgcattaacatgtggcagcggatcggccaggctatgtacgcccctcccatacagggcgttatccgttgcgtgagcaatattacaggcttgattttaactagagacggcggttccacaaattctacaaccgaaaccttcagacccggcgggggcgacatgcgtgacaactggcgctcagaactctacaaatacaaagttgtgaaaattgagggaggcgtatggaaggacgctgaaaccaccctgttctgcgcaagcgacgccaaggcatatgaaactgagaaacacaacgtatgggccacacacgcttgtgtccccacagatcccaacccccaagagattcacctcgagggtgttacagaggaattcaacatgtggaaaaacaacatggtcgagcagatgcacacagacatcatatccctctgggatcaaagtctgaagccctgcgtgaaactgactggcggcggtagtgggggtggatcaggcggtggtagtgatattcaaatgacgcaaagtcctagcagtgtctctgccagtgtgggtgaccgagtcacaatcacctgtagagcttcacaggatattagtacctggttggcttggtatcaacaaaagcctggaaaggccccaaaactcctgatttacgccgcctcaacattgcaatcaggagtgccttctagatttagtgggagcgggtctgggaccgacttctcacttacaattaacagcctgcagccggaagattttgccacctattattgccaacaagctaactctttcttcacgttcggggggggaactaaagttgagattaaacgtacggtggctgcaccatctgtcttcatcttcccgccatctgatgagcagttgaaatctggaactgcctctgttgtgtgcctgctgaataacttctaccccagagaagccaaagtgcagtggaaggtggacaacgccctgcagagcggaaacagccaggaaagcgtgacagagcaggattccaaggattccacatacagcctgagcagcacactgacactgtccaaggccgactacgagaagcacaaggtgtacgcctgcgaagtgacacaccagggactgtcctcccctgtgacaaagagcttcaacagaggagaatgctga

SEQ ID NO: 63 - protein, 279 aa, synthetic construct
MGWSCIILFLVATATGVHSEVQLVQSGAEVKKPGATVKISCKASGYTFSDFYMYWVRQAPGKGLEWMGLIDPEDACTMYAEKFRGRVTITADTSTDTGYLELSSLRSEDTAVYYCAADPWELNAFNVWGQGTLVSVSGAAVTGPRVFPLSPAGCSTTSPTTLLGCLVDDYYPLPVTVTWNGGALTSGVTTFPAVLRPDGLYSQVSVLSVPSSSCGTETYTCNVTHPASNTTVSQLVPCPRQLELPAEALAHLRAFLALLPPAAALAVAHAVLQALAAAL

SEQ ID NO: 64 - DNA, 840 bp, synthetic construct
  source 1..840: mol_type = other DNA
atgggctggtcctgcattatcctgttcctggtagcgacagctacaggtgtccactctgaagtgcagcttgtccagagtggcgccgaagttaagaaacccggagccactgtgaagatatcttgtaaagctagcggatacactttcagtgatttttatatgtattgggtgaggcaagcccctggtaagggcctggagtggatgggcctcatcgacccagaagacgcctgcaccatgtacgctgagaagttccgaggacgcgtcacaattacagcagatacatctacggatacgggataccttgaacttagctccctgcggtctgaggataccgccgtgtattattgtgcagctgacccctgggaactcaacgcttttaacgtatggggccaaggaacactcgtatctgttagcggtgccgcggtgactgggccacgagtttttcccctgagcccagctggctgttctaccacatcaccaactacactcctgggttgcctggtcgacgactactaccccctgcccgttactgtaacctggaatggcggcgctctgacatccggcgtaacaactttccccgccgtcctgcggcctgacggcttgtacagccaggtgtccgtgctgagcgtcccctctagtagctgtggcacagagacctatacctgcaacgtgactcatccagcctccaacacaaccgtttcacagctggtgccctgcccaagacaactggaattgccggccgaggccctggcgcacctcagggcattcctggccttgcttccgccagccgctgcccttgcagtcgcacatgcggtactccaggcactcgcagccgctctttaa

SEQ ID NO: 65 - protein, 267 aa, synthetic construct
MGWSCIILFLVATATGVHSDIQMTQSPSSVSASVGDRVTITCRASQDISTWLAWYQQKPGKAPKLLIYAASTLQSGVPSRFSGSGSGTDFSLTINSLQPEDFATYYCQQANSFFTFGGGTKVEIKRTVVAPSVSIFPPSPEELASGRARVVCVASSFYPESLEVTWLVDGEPVSGRSFTSTSAKNPADNTYSLVSTLDLSTEEYLAHRRFECRVTHSGLSSPVTASILRVGDTLFTSVSSSDDGVFTVTGSPLMQLALDLYLEAQAA

SEQ ID NO: 66 - DNA, 804 bp, synthetic construct
  source 1..804: mol_type = other DNA
atgggatggtcatgtattatccttttcctggtagccactgccacaggagtccacagcgacatccagatgacccagtccccatcttccgtttctgctagtgtcggagatagagtgaccatcacctgcagggcctcacaggatataagtacgtggctggcctggtaccagcagaaacccgggaaggcccctaaattgctgatttacgccgcctccacactccaatctggggtgcctagccggttttccggcagtggcagcggcacggatttcagtctgaccatcaactcactacagcctgaggactttgccacatactactgccagcaggccaacagcttcttcacctttggcggaggcacaaaggttgagatcaagcgcaccgtcgtcgcccctagtgtgagcatcttcccccctagccctgaggagctggcatccggccgcgcaagggtcgtgtgtgtagcttcctccttttaccccgagtctctggaagttacctggctggtggacggagagccagtgagcggaaggtctttcacatctacttcagccaagaacccagcagacaatacctattcactggtctcaaccttggatctttctacagaagagtatctggcccataggcgctttgaatgtagagtgacacattcaggcctgagcagtcctgtcacagccagcatactgagggtcggggacaccctgttcacttctgtttcttcttctgatgatggagtgtttacagtcaccgggtctcctctgatgcagctggctctggatttgtacttggaggctcaggccgcctag

SEQ ID NO: 67 - protein, 284 aa, synthetic construct
MGWSCIILFLVATATGVHSEVQLVQSGAEVKKPGATVKISCKASGYTFSDFYMYWVRQAPGKGLEWMGLIDPEDACTMYAEKFRGRVTITADTSTDTGYLELSSLRSEDTAVYYCAADPWELNAFNVWGQGTLVSVSGAPVTGPEVFPLPPMSVGSGKPSATLGCLVKNFYPLPVEVTWNGGAVTEGVYVFPPVLRPDGRWSLVSVLRVPESTLGTETYVCNVRHPPANVTVSKEVKALPGVTLTQTPEDAALEAKLRKACPEAEAIIVGTLPNGATLVLVFVP

SEQ ID NO: 68 - DNA, 855 bp, synthetic construct
  source 1..855: mol_type = other DNA
atggggtggtcctgcattattctgtttttggtggctaccgccactggagtgcacagcgaagtacagcttgtgcaaagtggtgctgaggtgaaaaagccaggcgcaaccgtgaaaatcagctgtaaggcgagcggctataccttcagcgatttctacatgtactgggtgcggcaggcacctggtaaaggactggagtggatgggtctgatcgatccagaggacgcctgtactatgtacgcggaaaaatttaggggtagagtgaccattaccgcagatacctcaactgatactggctatctggaactttcaagtttgcgcagcgaagataccgctgtctactattgtgcagctgacccctgggaacttaacgcattcaacgtgtgggggcagggaacattggtctcagtctccggcgctcccgtgaccgggcccgaggtgtttccactgccacctatgagcgtaggtagcggtaagccaagcgcaaccctggggtgcctcgttaagaacttttatccactgcctgtcgaagttacctggaatggcggcgctgtcacagaaggtgtgtatgtgttccctccggtattgcggccagatggcagatggagtctcgtcagcgtgctgagggttccggagtccactcttggaacggagacatacgtgtgtaacgtgcgacatcctccagcgaatgttacagtgagcaaagaagtgaaggctctaccaggtgtaactctcacacagacccctgaggatgctgcactggaagctaagttgaggaaggcctgccctgaagcagaagcaattatagtggggacattgcctaacggcgcgaccctggtcttggtgtttgtgccctga

SEQ ID NO: 69 - protein, 282 aa, synthetic construct; identical to SEQ ID NO: 31

SEQ ID NO: 70 - DNA, 849 bp, synthetic construct
  source 1..849: mol_type = other DNA
atgggatggtcttgcatcattctgtttctggtggctaccgctactggggtacacagcgatatacagatgacccaatctcccagttccgtgtccgcctccgtcggagatcgtgtgactattacatgccgcgctagccaagacatcagtacatggctagcatggtaccagcagaagcccgggaaagcacccaagcttctgatctatgcagcatccaccctgcagtcaggagtgcccagccgcttttccggaagcgggagtggaaccgatttttctcttacgatcaactctctacagcccgaagacttcgccacctattattgtcagcaggcgaacagttttttcacatttggtggcggcactaaggtggaaatcaaacgtacagtggctgctccgtccgtgttcatcttccccccatcagatgagcagctgaaatctggcaccgctagcgtggtctgtcttctgaacaatttctaccctcgcgaggctaaagtccagtggaaagttgacaacgctctgcaaagcggtaattcccaggaatctgttacagagcaggactccaaggatagcacatattccctgagtagtaccctgacactcagcaaggccgactacgagaagcataaggtttacgcgtgtgaagtaacccaccagggtttgagtagccccgttaccaaaagtttcaataagtatccgggcgtggtggttactgaaaccgtggacgaggcaacaggtaccactactattactcttaacacgaagaactacaaggcagtccgtctcgccataaagatagccgaggctctgcataagctcaggccaggttctactactgttgctgtcatttcttga

SEQ ID NO: 71 - protein, 650 aa, synthetic construct
LEAKAKALKAALDAAAAAAAAAAAAYLALLAATEAAVAAALAKIPDPATRAARAALAAAFAAALRGFATDLIAGGYAGLKERAEEIVELEKERAALAARRADLAAQAAALAALEAEGTRLQRELRGYLAAIKGMQEASMKLTESLHEVYEPDWYGREDVKMVGEKCDVLWEDFHQKLVDESLLTLDTYLGQFPALRARLAALAAQAAALEAALQALIDLLQKKPYGDAETTLFCASDAKAYETEKHNVWATHACVPAVPRFETPALPVTEEFNMWKNNMVEQMHTDIISLWDQSLKPCVKLTPLCNCNTSAITQACPKVSFEPIPIHYCAPAGFAILKCKDKKFNGTGPCPSVSTVQCTHGIKPVVSTQLLLNGSLAEEEVMIRSENITNNAKNILVQFNTPVQINCTRPNDIIGDIRQAHCNVSKATWNETLGKVVKQLRKHFGNNTIIRFANSSGGDLEVTTHSFNCGGEFFYCNTSGLFNSTWISNNDSITLPCRIKQIINMWQRIGQAMYAPPIQGVIRCVSNITGLILTRDGGSTNSTTETFRPGGGDMRDNWRSELYKYKVVKPLSEDPELKAARAAYAAARAAYDAAYDALAAELPALLSRRVGFYVNTFKNVSSLEAKFHKEIAVLCHKLYEVMTKLGDQHA

SEQ ID NO: 72 - protein, 614 aa, synthetic construct
EETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPREEECSPVLREGLGSLHTRHHCQIPVPDPATHGQYIVSVQPRRAEKHIKSSVNPTYYETFPNYQVIYLPTTTTDPSNKEPLGNTVTPAPVGTVTTYTPDGTRSSAITQACPKVTFEPIPIHYCAPAGFAILKCKDEEFNGTGLCKNVSTVQCTHGIKPVVSTQLLLNGSLAEKEIRIKSENISDNAKTIIVQLTKPVLINCARPSGDIRQAYCVVNRTQWNDTLGQVAIQLRKHWNTTIIFNEPSGGDLEITTHSFNCGGEFFYCNTSDLFNSTWNDNITLPCRIKQIINMWQRVGQAMYAPPIQGVIRCQSNITGILLTRDGGNTRTNETFRPGGGDMRDNWRSELYKYKVVKIE